(19) United States
(12) Patent Application Publication
(10) Pub. No.: US 2007/0026015 A1
(43) Pub. Date: Feb. 1, 2007

(54) Method

(75) Inventors: Ralph Patrick Braun, Highland Mills, NY (US); Lichun Dong, Kenmore, WA (US)

Correspondence Address: FOLEY AND LARDNER LLP;SUITE 500, 3000 K STREET NW, WASHINGTON, DC, 20007, US

(73) Assignee: Poweredject Vaccines, Inc.

(21) Appl. No.: 10/574,922

(22) PCT Filed: Oct. 12, 2004
(86) PCT No.: PCT/US04/33391

§ 371 Date: May 22, 2006

Related U.S. Application Data
(60) Provisional application No. 60/510,086, filed on Oct. 10, 2003.
(60) Provisional application No. 60/526,571, filed on Dec. 4, 2003.
(60) Provisional application No. 60/567,771, filed on May 5, 2004.

(51) Int. Cl. A61K 48/00; A61K 39/21; A61K 9/14

(52) U.S. Cl. 424/208.1; 514/44; 424/489

(57) ABSTRACT

The invention relates to a method of eliciting a T cell response against a T cell epitope in a host mammalian subject, which method comprises: (i) a first immunisation that comprises at least two administrations which are from 1 to 14 days apart to the subject, wherein each administration comprises administering a nucleotide of interest (NOI) encoding the T cell epitope, and optionally (ii) a second immunisation that comprises at least one administration to the subject of (a) a NOI encoding the T cell epitope, or (b) a protein comprising the T cell epitope, wherein the time between the first administration of the first immunisation, and the first administration of the second immunisation, is from 21 to 365 days.

### CROSS-REFERENCE TO RELATED PATENT APPLICATIONS

This application claims the benefit under 35 USC 119(e) to U.S. application Ser. Nos. 60/510,086, filed Oct. 10, 2003, 60/526,571, filed Dec. 04, 2003, and 60/567,771, filed May 05, 2004, which are all incorporated herein by reference.

### FIELD OF THE INVENTION

The present invention relates a method of eliciting an immune response.

### BACKGROUND OF THE INVENTION

Vaccination methods are described in the art, for example see Prayaga et al ((1997) Vaccine 15(12-13): 1349-1352), Kilpatrick et al (1997) Hybridoma 16: 381-389, Kilpatrick et al (1998) Hybridoma 17: 569-576, Pertmer et al (1995) Vaccine 13; 1427-1430 and Olsen et al (1997) Vaccine 15; 1149-1156. However, there remains a need for optimisation of nucleic acid administration schedules, including those that specifically induce an enhanced cell mediated immune (CMI response. This would be very beneficial for the prevention and treatment of a wide range of immune, inflammatory and infectious diseases and disorders.

### SUMMARY OF THE INVENTION

The present invention provides a method for eliciting (or enhancing) a CMI response in vivo. In particular the method elicits (or enhances) a T cell response in vivo.

Accordingly the invention provides a method of eliciting an immune response against a T cell epitope in a host mammalian subject, which method comprises:
(i) a first immunisation that comprises at least two administrations which are from 1 to 14 days apart to the subject, wherein each administration comprises administering a nucleotide of interest (NOI) encoding the T cell epitope, and optionally
(ii) a second immunisation that comprises at least one administration to the subject of (a) a NOI encoding the T cell epitope, or (b) a protein comprising the T cell epitope, wherein the time between
the first administration of the first immunisation, and
the first administration of the second immunisation, is from 21 to 365 days.

### ASPECTS OF THE INVENTION

The disclosure below discusses sequences, such as an epitope or antigen, which are encoded by the NOI which is administered. It is understood that instead of such an NOI, in the case of the second and subsequent immunisations a protein comprising the same epitope or antigen may be administered.

The invention relates to a method of eliciting a T cell response against a T cell epitope of interest (EOI). As mentioned above the method comprises the administration of a NOI which encodes the EOI. In the method at least 2, 4, 6, 10 or 20 or more (up to and including, for example, 40) different NOI's may be administered, wherein each of the NOI's encode the same epitope. Alternatively at each administration of NOI the same NOI may be administered. Similarly in embodiments where a protein comprising the EOI is administered it is understood that at least 2, 4, 10 or more (up to an including, for example 20) different proteins may be administered which comprise the epitope. Alternatively at each administration of protein the same protein (which comprises the epitope) may be administered.

In one aspect, the present invention provides a method of eliciting an enhanced cellular mediated immune (CMI) response against at least one target antigen (TA) in a host mammalian subject; wherein the method comprises administering a nucleotide sequence of interest (NOI) encoding one or more epitopes of interest (EOI) of the TA at least twice to the host mammalian subject; wherein the intervals between each NOI administrations ranges from about 48 hours to about 144 hours; and wherein the method is effective to provide an enhanced CMI response against the or each expressed EOI in the host mammalian subject.

The invention may be used prophylactically and/or therapeutically to immunomodulate the CMI response to one or more epitopes of interest (EOI) of a target antigen (TA). The time course of the induced response enables an effective strategy to be developed for immunotherapy of pre-existing T cell mediated disorders as well as facilitating broad protection against subsequently encountered antigens.

A further advantage of this aspect of the invention is the ability to enhance the CMI response without the use of an associated biological response modifier and/or an adjuvants.

The method of the invention may result in the production of an activated T cell. Numerous potential uses of activated T-cells are envisioned. For example, in the case of human therapy, it is contemplated that activated T-cells may be isolated, cultured ex vivo and administered to a host subject for the treatment of T cell mediated immune disorders and/or viral infections or patients with cancer. T-cells may be prepared by administering the NOI in vivo and then isolating the T-cells to expand in vitro in the presence of appropriate biological response modifiers and/or immunomodulators and/or adjuvants such as but not limited to peptide, cytokines and antigen presenting cells.

The NOI that is used in the method of the invention includes but is not limited to a DNA sequence under the control of a regulatory sequence which directs the expression of the DNA sequence in a mammalian host cell. The NOI encodes the T cell epitope, and thus typically encodes a protein which comprises the epitope. Thus preferably the NOI is capable of expressing the epitope (including a protein comprising the epitope) in a cell of the subject.

In preferred embodiments, the T cell epitope may be a helper T cell and/or a CD8+T lymphocyte (CD8+T cell) epitope. Thus the T cell response elicited by the method of the invention may a helper and/or CD8+T cell response. Even more preferably, the response may be a CD8+T lymphocyte response, such as a cytotoxic response.

The Adminstration Schedule

The method of the invention comprises sets of administrations which are referred to herein as “immunisations”. Thus the method may comprise 1, 2, 3, 4, 5 or more (for example up to and including 10) such sets of immunisations which are referred to as the “first immunisation”, “second immunisation” etc. herein.

One or more, or all, of the administrations of any of the immunisations (for example the first and/or second or all immunisations) may occur over from 2 to 14 days (i.e. the first and last administration of the immunisation are within from 2 to 14 days of each other), such as from 3 to 12 days or 4 to 8 days. Preferably the first immunisation occurs over from 2 to 14 days, such as from 3 to 12 days or 4 to 8 days.

One or more, or all, of the immunisations may comprise from 2 to 50, such as from 5 to 40 or 10 to 30 administrations. Thus typically in one or more, or all, of the immunisations at least 2, such as at least 3, 5, 10, 30, 50 or more (for example up to and including 100 administrations) administrations may be given. Preferably the first immunisation (and optionally one or more subsequent immunisations) comprises from 3 to 20 administrations. In one embodiment the method (i.e. all of the immunisations together) comprises 3 to 50, such as from 5 to 40 or 10 to 30 administrations.

In one embodiment more than one administration (typically from 2 to 5 administrations) may be given at the same time point (such as on the same day, or within a day of each other, within twelve hours of each other, within two hours of each other or within an hour of each other). As will be discussed below such administrations which are given at the same time point may be given to the same or to different sites.

One or more, or all, of the immunisations may comprise administrations at 2 to 10, such as 3 to 5, different time points, wherein such time points are preferably on different days. Thus one or more, or all, of the immunisations may comprise administrations on 2 to 10, such as 3 to 5, different days. Preferably in the first and second immunisations administrations occur on 3 or 4 different days.

In the case of one or more, or all, of the immunisations 2, 3, 4 or more of the administrations of that immunisation may be from 2 to 14 days apart, such as from 3 to 10 or from 4 to 8 days apart. Preferably for one or more, or all, of the immunisations 2, 3, 4 or more of the administrations of that immunisation are from 2 to 6 days apart.

The time between two immunisations is defined herein as the time between the first administrations of the two immunisations. Typically the time between the first and second immunisation (and preferably the time between all the immunisations) is from 21 to 365 days, such as from 28 to 300 days, 50 to 250 days or 100 to 200 days. In one embodiment all of the immunisations of the method are carried out over 21 to 365 days, such as from 28 to 300 days, 50 to 250 days or 100 to 200 days.

In one embodiment of the method of the invention, the NOI is generally administered 2 to 5 times (at 2 to 5 different time points), such as 2, 3 or 4 times (at 2, 3 or 4 different time points respectively). Typically such administrations are over 2 to 14 days, for example over 4 to 12 or 6 to 10 days. In a preferred embodiment NOI at least 2, 3 or 4 administrations of NOI are carried out which may be separated by 3 days or less, such as 2 days or less. In one embodiment the time between the first and second administrations is less than 4 days, typically less than 3.5 days, such as 3 days or less, or 2 days or less.

Preferably NOI is not administered to the subject in-between the administrations discussed herein, and typically other products which may stimulate an immune response (such as a polypeptide antigen), are not administered in-between the said NOI administrations. In one embodiment NOI, or another product which may stimulate an immune response (such as a polypeptide antigen), is not administered to the subject at least 7 days, such as at least 14 or at least 28 days before the first administration of NOI in any of the administration regimens mentioned herein. In one embodiment NOI, or another product which may stimulate an immune response (such as a polypeptide antigen), is not administered to the subject at least 7 days, such as at least 14 or at least 28 days after the last administration of NOI in any of the administration regimens mentioned herein.

Typically about 1 pg to about 5 mg of NOI provided to the subject in each administration in which NOI is administered (or at each time point), preferably from about 10 pg to about 100 ug, such 25 pg to 1 ug or 50 pg to about 500 pg. As mentioned above in the second and, if applicable, subsequent immunisations a protein may be administered. Typically about from 0.1 ug to 20 mg of protein is administered in each administration (or at each time point), preferably from 1 ug to 5 mg, such as 10 ug to 500 ug.

As mentioned above in the method of the invention NOI and optionally also protein is administered. For some embodiments, the NOI or protein is co-administered with an adjuvant or an NOI encoding same. In this embodiment, the adjuvant is preferably the non-toxic form of the E.coli heat-labile enterotoxin (LT) or the Vibrio Cholerae cholera toxin (CT). The adjuvant may comprise the A or B subunit of the LT enterotoxin (LTB) or the B subunit of the CT Cholera toxin (CTB).

The inclusion of an adjuvant and in particular, a genetic adjuvant is useful in further enhancing or modulating the CMI response. Thus the method of the present invention for enhancing a CMI response may be refined, by the addition of adjuvants to the NOI or protein (or compositions comprising the NOI or protein) which lead to particularly effective compositions and methods for eliciting a long lived and sustained enhanced CMI response.

The NOI or protein is preferably administered as a particle. In a preferred embodiment of the method of the present invention, the NOI or protein is administered transdermally. In an even more preferred embodiment, the particle is administered to the host mammalian subject by a particle acceleration device.

In one embodiment after the administration regimen has been carried out, whether or not the regimen has led to the stimulation of a CMI (such as a CTL response) is ascertained. This can be done for example by measuring the presence of, or the level of, T cells (such as CTL) in a sample from the subject. The T cells which are detected are generally specific to an epitope encoded by the NOI.

Other aspects of the present invention are presented in the accompanying claims and in the following description and drawings. These aspects are presented under separate section headings. However, it is to be understood that the teachings under each section are not necessarily limited to that particular section heading.

### Definitions

It is to be understood that this invention is not limited to particularly exemplified molecules or process parameters as such may, of course, vary. It is also to be understood that the terminology used herein is for the purpose of describing particular embodiments of the invention only, and is not intended to be limiting. In addition, the practice of the present invention will employ, unless otherwise indicated, conventional methods of virology, microbiology, molecular biology, recombinant DNA techniques and immunology all of which are within the ordinary skill of the art. Such techniques are explained fully in the literature. See, e.g., Sambrook, et al., Molecular Cloning: A Laboratory Manual (2nd Edition, 1989); DNA Cloning: A Practical Approach, vol. I & II (D. Glover, ed.); Oligonucleotide Synthesis (N. Gait, ed., 1984); A Practical Guide to Molecular Cloning (1984); and Fundamental Virology, 2nd Edition, vol. I & II (B. N. Fields and D. M. Knipe, eds.).

All publications, patents and patent applications cited herein, whether supra or infra, are hereby incorporated by reference in their entirety. It must be noted that, as used in this specification and the appended claims, the singular forms “a”, “an” and “the” include plural referents unless the content clearly dictates otherwise. All scientific and technical terms used in this application have meanings commonly used in the art unless otherwise specified. As used in this application, the following words or phrases have the meanings specified.

Immune Responce

The mechanism by which the immune system controls disease includes the induction of neutralising antibodies via humoral immunity and the generation of T-cell responses via cellular immunity. As used herein, the term “immune response” against a target antigen (TA) (including EOI) refers to the development in a host mammalian subject of a humoral and/or a cellular immune response against that TA.

As used herein, the term “humoral immune response” refers to an immune response mediated by antibody molecules. The antibodies generated by humoral immunity are primarily effective against extracellular infectious agents.

As used herein, the term “cell mediated immune (CMI) response” is one mediated by T-lymphocytes and/or other white blood cells. The CMI immune mechanisms are generally more effective against intracellular infections and disease because the CMI mechanisms prime T cells in a way that, when a TA appears at a later date, memory T cells are activated to result in a CMI response that destroys target cells that have the corresponding TA or a portion thereof on their cell surfaces, and thereby the infecting pathogen. The CMI response is focused on the destruction of the source of infection mediated by either effector cells that destroy infected cells of the host by direct cell-to-cell contact and/or by the release of molecules, such as cytokines, that possess anti-viral activity. Thus the CMI response, which is characterised by a specific T lymphocyte cellular response, is crucial to produce resistance to diseases caused by cancer, viruses, pathogenic and other intracellular microorganisms.

T Cells Implicated in the CMI Responce

At least two special types of T cells are required to initiate and/or to enhance CMI and and humoral responses. The antigenic receptors on a particular subset of T cells which express a CD4 co-receptor can be T helper (Th) cells or CD4 T cells (herein after called T helper cells) and they recognise antigenic peptides bound to MHC class II molecules. In contrast, the antigenic receptors on a particular subset of T cells which express a CD8 co-receptor are called cytotoxic T lymphocytes (CTLs) or CD8+T cells (hereinafter called CD8+T cells) and they react with antigens displayed on MHC Class I molecules.

Helper T Cells

Helper T cells or CD4+cells can be further divided into two finctionally distinct subsets: Th1 and Th2 which differ in their cytokine and effector fumction. Tm1 and Th2 responses are regulated not only in a positive but also in a negative way such that Th1 cellular responses are augmented by Th1 cytokines such as IL-2, IL-12 and IFN-gamma and decreased by Th2 cytokines such as IL-4 and IL-10. In contrast, antibody responses are enhanced by Th2 cytokines such as IL-4 and IL-10 but are downregulated by Th1 cytokines such as IFN-gamma and another cytokine IL-12 that enhances IFN-gamma and is produced by monocytes. Thus, classic Th1 cytokines such as IFN-gamma, IL-2 and IL-12 can be regarded as immune co-factors that induce an inflammatory response. In contrast, the classic Th2 cytokines such as IL-4 and IL-10 can be regarded as cytokines that will suppress a severe inflammatory response in some situations.

CD8+T Cells

CD8+T cells may fimction in more than one way. The best known function of CD8+T cells is the killing or lysis of target cells bearing peptide antigen in the context of an MHC class I molecule. Hence the reason why these cells are often termed cytotoxic T lymphocytes (CTL). However, another function, perhaps of greater protective relevance in certain infections is the ability of CD8+T cells to secrete interferon gamma (IFN-gamma). Thus assays of lytic activity and of IFN-gamma release are both of value in measuring CD8+T cell immune response (e.g. in an ELISPOT assay as set forth below). In infectious diseases there is evidence to suggest that CD8+T cells can protect by killing an infectious agent comprising an infectious antigen at the early stages of a disease before any symptoms of disease are produced.

Enhanced CMI Responce

The present invention concerns a method capable of enhancing and/or modulating the CMI response in a host subject against a target antigen. As used herein, the term “enhancing” encompasses improvements in all aspects of the CMI response which include but are not limited to a stimulation and/or augmentation and/or potentiation and/or up-regulation of the magnitude and/or duration, and/or quality of the CMI response to a repeatedly administered NOI encoding an EOI of a TA. By way of example, the CMI response may be enhanced by either (i) enhancing the activation and/or production and/or proliferation of CD8+T cells that recognise a target antigen and/or (ii) shiffing the CMI response from a Th2 to a Th1 type response. This enhancement of the Th1 associated responses is of particular value in responding to intracellular infections because, as explained above, the CMI response is enhanced by activated Th1 (such as, for example, IFN-gamma inducing) cells.

Such an enhanced immune response may be generally characterized by increased titers of interferon-producing CD4⁺and/or CD8⁺T lymphocytes, increased antigen-specific CD8+T cell activity, and a T helper 1-like immune response (Th1) against the antigen of interest (characterized by increased antigen-specific antibody titers of the subclasses typically associated with cellular immunity (such as, for example IgG2a), usually with a concomitant reduction of antibody titers of the subclasses typically associated with humoral immunity (such as, for example IgG1)) instead of a T helper 2-like immune response (Th2).

The response which is elicited by the method of the invention (e.g. the enhancement of a CMI response) may be determined by a number of well-known assays, such as by lympho-proliferation (lymphocyte activation) assays, CD8+T cell assays, or by assaying for T-lymphocytes specific for the epitope in a sensitized subject (see, for example, Erickson et al. (1993) J. Immunol. 151: 4189-4199; and Doe et al. (1994) Eur. J. Immunol. 24: 2369-2376) or CD8+T cell ELISPOT assays for measuring Interferon gamma production (Miyahara et al PNAS(USA) (1998) 95: 3954-3959).

In one embodiment the method elicits a regulatory or suppressor T cell response. Eliciting such a response may be used, for example, to prevent or treat an autoimmune disease.

Enhanced T-Cell Response

In the disclosure herein eliciting a response is often discussed in terms of eliciting a CMI response. The eliciting of a CMI response is understood to include eliciting of a T cell response. The response which is elicited by the method of the invention may be an “enhanced” response. An “enhanced” response may be said to occur if the response elicited by the method of the invention is more than the response which is elicited in a control method, where in the control method the same amount of NOI (and if applicable also the same amount of protein) has been administered as was administered in the method of the invention. Such a control method may for example consist of administration of the NOI (and if applicable also protein) in a single administration. Alternatively the control method may consist of administration of the NOI (and if applicable also protein) in two separate administrations which are 28 days apart.

As used herein, the term “enhancing a T -cell response” encompasses improvements in all aspects of the T-cell response which include but are not limited to a stimulation and/or augmentation and/or potentiation and/or up-regulation of the magnitude and/or duration, and/or quality of the T-cell response to a repeatedly administered NOI encoding an EOI of a target antigen. By way of example, the T-cell response may be enhanced by either enhancing the activation and/or production and/or distribution and/or proliferation of the induced T-cells and/or longevity of the T-cell response to T-cell inducing/modulating NOIs encoding EOIs from a TA. The enhancement of the T-cell response in a host subject may be associated with the enhancement and/or modulation of the Th1 immune response in the host subject.

The enhancement of the T-cell response may be determined by a number of well-known assays, such as by lympho-proliferation (lymphocyte activation) assays, CD8+T-cell cytotoxic cell assays, or by assaying for T-lymphocytes specific for the epitope in a sensitized subject (see, for example, Erickson et al. (1993) J. Immunol. 151: 4189-4199; and Doe et al. (1994) Eur. J. Immunol. 24: 2369-2376) or CD8+T-cell ELISPOT assays for measuring Interferon gamma production (Miyahara et al PNAS(USA) (1998) 95: 3954-3959).

Antigen

Each disease causing agent or disease state has associated with it an antigen or immunodominant epitope on the antigen which is crucial in immune recognition and ultimate elimination or control of a disease causing agent or disease state in a host. In order to mount a humoral and/or cellular immune response against a particular disease, the host immune system must come in contact with an antigen or an immunodominant epitope on an antigen associated with that disease state.

As used herein, the term “antigen” refers to any agent, generally a macromolecule, which can elicit an immunological response in an individual. The immunological response may be of B- and/or T-lymphocytic cells. The term may be used to refer to an individual macromolecule or to a homogeneous or heterogeneous population of antigenic macromolecules. As used herein, “antigen” is used to refer to a protein molecule or portion thereof which contains one or more antigenic determinants or epitopes.

Target Antigen

As used herein, the term “target antigen (TA)” means an immunogenic peptide or protein of interest comprising one or more epitopes capable of inducing a CMI response to an infectious pathogen such as but not limited to a bacteria, viruses, fungi, yeast, parasites and other microorganisms capable of infecting mammalian species. The target antigen can include but is not limited to an auto-antigen, a self-antigen, a cross-reacting antigen, an alloantigen, a tolerogen, an allergen, a hapten, an immunogen or parts thereof as well as any combinations thereof. Thus the EOI may be from any of the types of antigens or proteins mentioned herein or from any of the specific antigens or proteins mentioned herein.

Epitope

As used herein, the term “epitope” generally refers to the site on a target antigen which is recognised by a T-cell receptor and/or an antibody. Preferably it is a short peptide derived from or as part of a protein antigen. However the term is also intended to include peptides with glycopeptides and carbohydrate epitopes. A single antigenic molecule may comprise several different epitopes. The term “epitope” also includes modified sequences of amino acids or carbohydrates which stimulate responses which recognise the whole organism. It is advantageous if the selected epitope is an epitope of an infectious agent (such as a bacterium or virus) which causes the infectious disease.

As used herein, the term epitope of interest (EOI) refers to one or more EOI which may be used in the method of the invention. The method of the invention may be used elicit a T cell response to 1, 2, 3, 4, 5 to 10 or more different epitopes. Thus the method may comprise administration of one or more NOI's which together encode 1, 2, 3, 4, 5 to 10 or more different epitopes and/or administration of one or more proteins which together comprise 1, 2, 3, 4, 5 to 10 or more different epitopes.

In one embodiment the method of the invention is carried out to elicit a T cell response to a predetermined (or predefined) and/or known epitope, which is typically from a predetermined and/or known protein.

Source of Epitopes

The EOI can be generated from knowledge the amino acid and corresponding DNA sequences of the peptide or polypeptide, as well as from the nature of particular amino acids (e.g., size, charge, etc.) and the codon dictionary, without undue experimentation. See, e.g., Ivan Roitt, Essential Immunology, 1988; Kendrew, supra; Janis Kuby, Immunology, 1992 e.g., pp. 79-81. Some guidelines in determining whether a protein or an epitope of interest which will stimulate a response, include: Peptide length—the peptide should be at least 8 or 9 amino acids long to fit into the MHC class I complex and at least 8-25, such at least as 13-25 amino acids long to fit into a class II MHC complex. This length is a minimum for the peptide to bind to the MHC complex. It is preferred for the peptides to be longer than these lengths because cells may cut peptides. The peptide should contain an appropriate anchor motif which will enable it to bind to the various class I or class II molecules with high enough specificity to generate an immune response (See Bocchia, M. et al, Specific Binding of Leukemia Oncogene Fusion Protein Pentides to HLA Class I Molecules, Blood 85:2680-2684; Englehard, VH, Structure of peptides associated with class I and class II MHC molecules Ann. Rev. Immunol. 12:181 (1994)). This can be done, without undue experimentation, by comparing the sequence of the protein of interest with published structures of peptides associated with the MHC molecules. Thus, the skilled artisan can ascertain an epitope of interest by comparing the protein sequence with sequences listed in the protein database.

The method of the present invention is generally applicable to enhancing the CMI response against NOIs encoding EOIs from any source (for example from a pathogen), including those from a wide variety of infectious agents such as viruses or parasites. By way of 15 example, the EOI may be derived from pathogenic agents derived from tumour cells which multiply unrestrictedly in an organism and may thus lead to pathological growths. Examples of such pathogenic agents are described in Davis, B. D. et al (Microbiology, 3rd ed., Harper International Edition).

The epitope may be from a non-mammalian, non-mouse or non-human protein. The epitope may be from an intracellular protein or an extracellular protein. In one embodiment the epitope is from a secreted protein, such as a protein secreted by a pathogen. The epitope may or may not be from a protein of the subject in whom the T cell response is being elicited. The epitope may be from a pathogen which is able to infect the subject. The epitope may be a naturally occurring epitope or an artificial epitope which is not found in nature.

However, in preferred embodiments, the invention is exemplified by enhancing the CMI response against components of the HIV viral family. Enhanced CMI responses may be generated against EOIs located within the products of any viral gene, such as, for example, the gag, pol, nef and env genes, with the products of the env genes being preferred targets.

Thus in one embodiment the method of the invention elicits an immune response against particular antigens for the treatment and/or prevention of HIV infection and/or any condition which is caused by or exacerbated by HIV infection, such as AIDS.

T Cell Epitopes

In the methods or processes of the present invention, the EOI of the TA may contain one or more T cell epitopes. As used herein, the term “T cell epitope” refers generally to those features of a peptide structure which are capable of inducing a T cell response. In this regard, it is accepted in the art that T cell epitopes comprise linear peptide determinants that assume extended conformations within the peptide-binding cleft of MHC molecules (Unanue et al. (1987) Science 236: 551-557). As used herein, a T cell epitope is generally a peptide having at least about 3-5 amino acid residues, and preferably at least 5-10 or more amino acid residues, such as 8 to 25 amino acid residues. However, as used herein, the term “T cell epitope” encompasses any MHC Class I-or MHC Class II restricted peptide. The ability of a particular T cell epitope to stimulate/enhance a CMI response may be determined by a number of well-known assays, such as by lymphoproliferation (lymphocyte activation) assays, CD8+ T-cell cytotoxic cell assays, or by assaying for T-lymphocytes specific for the epitope in a sensitized subject. See, e.g., Erickson et al. (1993) J. Inmunol. 151: 4189-4199; and Doe et al. (1994) Eur. J. Immunol. 24: 2369-2376 or CD8+T-cell ELISPOT assays for measuring interferon gamma production (Miyahara et al PNAS(USA) (1998) 95: 3954-3959).

CD8+T-Cell Epitopes

Preferably the EOI is a CD8+T-cell EOI. A CD8+T-cell -inducing EOI is an epitope capable of stimulating the formation, or increasing the activity, of specific CD8+T-cells following its administration to a host subject. The CD8+T-cell epitopes may be provided in a variety of different forms such as a recombinant string of one or two or more epitopes. CD8+T-cell epitopes have been identified and can be found in the literature, for many different diseases. It is possible to design epitope strings to generate CD8+T-cell response against any chosen TA that contains such CD8+T-cell EOIs. Advantageously, in the NOI CD8+T-cell EOIs may be provided in a string of multiple EOIs which are linked together without intervening sequences so that unnecessary nucleic acid material is avoided. In one embodiment the NOI also encodes sequence which can act as a protease cleavage sites to allow cleavage of the epitopes from the expressed protein.

T Helper Epitopes

Preferably the EOI is a helper T lymphocyte EOI. Various methods are available to identify T helper cell EOIs suitable for use in accordance herewith. For example, the amphipathicity of a peptide sequence is known to effect its ability to function as a T helper cell inducer. A full discussion of T helper cell-inducing epitopes is given in U.S. Pat. No. 5,128,319, incorporated herein by reference.

B Cell Epitopes

Preferably the EOI is a mixture of CD8+T-cell EOIs and B cell EOIs. As used herein, the term “B cell epitope” generally refers to the site on a TA to which a specific antibody molecule binds. The identification of epitopes which are able to elicit an antibody response is readily accomplished using techniques well known in the art. See, e. g., Geysen et al. (1984) Proc. Natl. Acad. Sci. USA 81: 3998-4002 (general method of rapidly synthesizing peptides to determine the location of immunogenic epitopes in a given antigen); U.S. Pat. No. 4,708,871 (procedures for identifying and chemically synthesizing epitopes of antigens); and Geysen et al.(1986) Molecular Immunology 23: 709-715 (technique for identifying peptides with high affinity for a given antibody).

Combination of Epitopes

In a preferred embodiment of the present invention, the EOI is a mixture of a CD8+T-cell—inducing EOI and a T helper cell-inducing EOI.

As is well known in the art, T and B cell inducing epitopes are frequently distinct from each other and can comprise different peptide sequences. Therefore certain regions of a protein's peptide chain can possess either T cell or B cell epitopes. Therefore, in addition to the CD8+T-cell epitopes, it may be preferable to include one or more epitopes recognised by T helper cells, to augment the immune response generated by the CD8+T-cell epitopes.

The mechanism of enhancing a CD8+T-cell induced response in vivo by T helper cell inducing agents is not completely clear. However, without being bound by theory, it is likely that the enhancing agent, by virtue of its ability to induce T helper cells, will result in increased levels of necessary cytokines that assist in the clonal expansion and dissemination of specific CD8+T-cells. Regardless of the underlying mechanism, it is envisioned that the use of mixtures of helper T cell and CD8+T-cell —inducing EOIs in the methods of the present invention will assist in the enhancement of the CMI response. Particularly suitable T helper cell epitopes are ones which are active in individuals of different HLA types, for example T helper epitopes from tetanus (against which most individuals will already be primed). It may also be useful to include B cell EOIs for stimulating B cell responses and antibody production. Synthetic NOIs may also be constructed to produce two types of immune responses: T cell only and T cell combined with a B cell response.

Immunodominant Epitope

When an individual is immunized with an NOI encoding multiple EOIs of a TA, in many instances the majority of responding T lymphocytes will be specific for one or more linear EOIs from that TA and/or a majority of the responding B lymphocytes will be specific for one or more linear or conformational EOIs from that TA. For the purposes of the present invention, then, such EOIs are referred to as “immunodominant epitopes”. In an antigen having several immunodominant EOIs a single EOI may be the most dominant in terms of commanding a specific T or B cell response.

Preferably the method or process of the present invention is effective in enhancing a CMI response against one or more HSV-2 epitopes. Preferably the method or process of the present invention is effective in enhancing a CMI response against one or more immunodominant HSV-2 epitopes. Preferably the method of the present invention is effective in generating/enhancing a CMI response against one or more HbsAg epitopes. Preferably the method of the present invention is effective in generating/enhancing a CMI response against one or more immunodominant HbsAg epitopes.

As the Examples show, the enhanced CMI response obtained against both HSV-2 and HbsAg EOIs indicates that the method of the present invention is generally effective against EOI from diverse infectious pathogens. Moreover the protective effect against viral challenge indicates that the generation of a strong CD8+T-cell response is of value in the development of preventative and therapeutic vaccination strategies.

Preferably the methods or processes of the present invention are effective in eliciting an enhanced CMI response against one or more EOIs associated with a tumour associated antigen (TAA). Advantageously, EOIs derived from tumour associated antigens (TAA) can serve as targets for the host immune system and elicit responses which result in tumour destruction. Examples of such TAAs include but are not limited to MART-1 (Melanoma Antigen Recognised by T cells-1) MAGE-1, MAGE-3, 5T4, gp100, Carcinoembryonic antigen (CEA), prostate-specific antigen (PSA), MUCIN (MUC-1), tyrosinase. Other TAAs may be identified, isolated and cloned by methods known in the art such as those disclosed in U.S. Pat. No. 4,514,506.

In a preferred embodiment the NOI encodes at least two HIV antigens. The NOI may comprise a sequence encoding HIV gag protein, or fragment thereof containing an epitope, and one or more further HIV antigens or fragment thereof containing an epitope. The antigens may derive from any available HIV isolates (typically HIV-1), such as HXB2. The antigens may include gag antigens (or fragments thereof which contain an epitope) such as p24gag and p55gag, as well as proteins derived from the pol, env, tat, vif, rev, nef, vpr, vpu and LTR regions of HIV (or fragments thereof which contain an epitope).

In a more preferred embodiment the NOI encodes at least three HIV antigens, preferably Gag, nef and RT (or instead of the whole protein a fragment of any of these proteins which contains an epitope). These coding sequences may be in any order, but are preferably in the order Nef-RT-Gag, RT-Nef, Gag or RT-Gag-Nef.

In one embodiment the HV epitopes/proteins which are expressed are fusion proteins, such as a fusion protein containing sequence from (including the above-mentioned fragments) Nef, RT and Gag.

In a preferred embodiment, the gag gene does not encode the gag p6 peptide. Preferably the nef gene in the NOI is truncated to remove the sequence encoding the N terminal 81 amino acids.

The fragments of gag, or any other HIV antigen (such as nef or RT), which are encoded by the NOI generally comprise an epitope. The proteins (including said fragments) encoded by the NOI are generally at least 8 amino acids long, for example 8-10 amino acids or up to 20, 50, 60, 70, 80, 100, 150 or 200 amino acids in length. Any such protein may be codon optimised, e.g. such that the fragment has a codon usage pattern which resembles that of a highly expressed mammalian gene.

In one embodiment the NOI encodes one of the following combinations of polypeptide:
I. p17, p24, fused to truncated NEF (devoid of nucleotides encoding terminal amino-acids 1-85).
II. p17, p24, RT, truncated NEF (devoid of nucleotides encoding terminal amino-acids 1-85).
III. p17, p24 (optimised gag) truncated NEF (devoid of nucleotides encoding terminal amino-acids 1-85).
IV. p17, p24 (optimised gag) RT (optimised) truncated NEF (devoid of nucleotides encoding terminal amino-acids 1-85).
V. p17, p24, RT (optimised) truncated NEF (devoid of nucleotides encoding terminal amino-acids 1-85).

In a preferred embodiment the NOI comprises inactivated codon optimised RT, truncated Nef and the p17/p24 portion of the codon optimised gag gene (for example as disclosed in WO 03/025003), optionally operatively linked downstream of an Iowa length HCMV promoter+exon 1 and/or upstream of a rabbit globin poly-adenylation signal. The poly-adenylation signal may be of rabbit beta globin gene.

In a preferred embodiment the NOI comprises any one or more of the polynucleotide sequences shown in FIGS. 18 to 22 or a fragment of such a sequence which encodes at least one epitope (preferably T cell epitope); or a homologue of any of the sequences of FIGS. 18 to 22 or a homologue of said fragment. Such a fragment or homologue is typically at least 50 nucleotides, such as at least 100, 200, 500 or 1000 nucleotides in length. In one embodiment the NOI is in the form of a plasmid as shown in any one of FIGS. 17 or 20 to 22 or a fragment or derivative (including a homologue) of such a plasmid. The construction of the plasmids is described in WO 03/080112 (incorporated herein by reference).

Optimised Condons

In a preferred embodiment of the invention the coding sequence of the NOI is optimised to resemble the codon usage of highly expressed genes in mammalian cells. The DNA code has 4 letters (A, T, C and G) and uses these to spell three letter “codons” which represent the amino acids the proteins encoded in an organism's genes. The linear sequence of codons along the DNA molecule is translated into the linear sequence of amino acids in the protein(s) encoded by those genes. The code is highly degenerate, with 61 codons coding for the 20 natural amino acids and 3 codons representing “stop” signals. Thus, most amino acids are coded for by more than one codon - in fact several are coded for by four or more different codons.

Where more than one codon is available to code for a given amino acid, it has been observed that the codon usage patterns of organisms are highly non-random. Different species show a different bias in their codon selection and, furthermore, utilization of codons may be markedly different in a single species between genes which are expressed at high and low levels. This bias is different in viruses, plants, bacteria and mammalian cells, and some species show a stronger bias away from a random codon selection than others.

For example, humans and other mammals are less strongly biased than certain bacteria or viruses. For these reasons, there is a significant probability that a mammalian gene expressed in E.coli or a viral gene expressed in mammalian cells will have an inappropriate distribution of codons for efficient expression. It is believed that the presence in a heterologous DNA sequence of clusters of codons which are rarely observed in the host in which expression is to occur, is predictive of low heterologous expression levels in that host.

In the NOI, the codon usage pattern may be altered from that found naturally to more closely represent the codon bias of the target organism, e.g. a mammal, especially a human. The “codon usage coefficient” is a measure of how closely the codon pattern of a given polynucleotide sequence resembles that of a target species. Codon frequencies can be derived from literature sources for the highly expressed genes of many species (see e.g. Nakamura et.al. Nucleic Acids Research 1996, 24:214-215). The codon frequencies for each of the 61 codons (expressed as the number of occurrences occurrence per 1000 codons of the selected class of genes) are normalised for each of the twenty natural amino acids, so that the value for the most frequently used codon for each amino acid is set to 1 and the frequencies for the less common codons are scaled to lie between zero and 1. Thus each of the 61 codons is assigned a value of 1 or lower for the highly expressed genes of the target species. In order to calculate a codon usage coefficient for a specific polynucleotide, relative to the highly expressed genes of that species, the scaled value for each codon of the specific polynucleotide are noted and the geometric mean of all these values is taken (by dividing the sum of the natural logs of these values by the total number of codons and take the anti-log). The coefficient will have a value between zero and 1 and the higher the coefficient the more codons in the polynucleotide are frequently used codons. If a polynucleotide sequence has a codon usage coefficient of 1, all of the codons are “most frequent” codons for highly expressed genes of the target species.

According to the present invention, the codon usage pattern of the NOI will preferably exclude codons with an RSCU value of less than 0.2 in highly expressed genes of the target organism. A relative synonymous codon usage (RSCU) value is the observed number of codons divided by the number expected if all codons for that amino acid were used equally frequently. The NOI will generally have a codon usage coefficient for highly expressed human genes of greater than 0.3, preferably greater than 0.4, most preferably greater than 0.5. Codon usage tables for human can also be found in GenBank. In comparison, a highly expressed beta action gene has a RSCU of 0.747. The codon usage table for a homo sapiens is set out below:

Homo sapiens [gbpri]: 27143 CDS's (12816923 codons)
fields: [triplet] [frequency: per thousand] ([number])

| | | | | | | | |
|---|---|---|---|---|---|---|---|
| UUU | 17.0(217684) | UCU | 14.8(189419) | UAU | 12.1(155645) | UGU | 10.0(127719) |
| UUC | 20.5(262753) | UCC | 17.5(224470) | UAC | 15.8(202481) | UGC | 12.3(157257) |
| UUA | 7.3(93924) | UCA | 11.9(152074) | UAA | 0.7(9195) | UGA | 1.3(16025) |
| UUG | 12.5(159611) | UCG | 4.5(57572) | UAG | 0.5(6789) | UGG | 12.9(165930) |
| CUU | 12.8(163707) | CCU | 17.3(222146) | CAU | 10.5(134186) | CGU | 4.6(59454) |
| CUC | 19.3(247391) | CCC | 20.0(256235) | CAC | 14.9(190928) | CGC | 10.8(137865) |
| CUA | 7.0(89078) | CCA | 16.7(214583) | CAA | 12.0(153590) | CGA | 6.3(80709) |
| CUG | 39.7(509096) | CCG | 7.0(89619) | CAG | 34.5(441727) | CGG | 11.6(148666) |
| AUU | 15.8(202844) | ACU | 12.9(165392) | AAU | 17.0(218508) | AGU | 12.0(154442) |
| AUC | 21.6(277066) | ACC | 19.3(247805) | AAC | 19.8(253475) | AGC | 19.3(247583) |
| AUA | 7.2(92133) | ACA | 14.9(191518) | AAA | 24.0(308123) | AGA | 11.5(147264) |
| AUG | 22.3(285776) | ACG | 6.3(80369) | AAG | 32.6(418141) | AGG | 11.3(145276) |
| GUU | 10.9(139611) | GCU | 18.5(236639) | GAU | 22.4(286742) | GGU | 10.8(138606) |
| GUC | 14.6(187333) | GCC | 28.3(362086) | GAC | 26.1(334158) | GGC | 22.7(290904) |
| GUA | 7.0(89644) | GCA | 15.9(203310) | GAA | 29.1(373151) | GGA | 16.4(210643) |
| GUG | 28.8(369006) | GCG | 7.5(96455) | GAG | 40.2(515485) | GGG | 16.4(209907) |

Coding GC 52.51% 1st letter GC 56.04% 2nd letter GC 42.35% 3rd letter GC 59.13%

Adjuvants

The method or process of the present invention does not require the presence of an adjuvant to demonstrate an enhanced CMI response. However, the inclusion of an adjuvant and in particular, a genetic adjuvant may be useful in further enhancing or modulating the CMI response. An adjuvant may enhance the CMI response by enhancing the immunogenicity of a co-administered antigen in an immunized subject, as well inducing a Th1 -like immune response against the co-administered antigen which is beneficial in a vaccine product.

Thus the methods or processes of the present invention for enhancing a CMI response may be refined, by the addition of adjuvants to the NOI or protein or compositions comprising the NOI or protein which lead to particularly effective compositions and methods for eliciting a long lived and sustained enhanced CMI response.

As used herein, the term “adjuvant” refers to any material or composition capable of specifically or non-specifically altering, enhancing, directing, redirecting, potentiating or initiating an antigen-specific immune response.

The term “adjuvant” includes but is not limited to a bacterial ADP-ribosylating exotoxin, a biologically active factor, immunomodulatory molecule, biological response modifier or immunostimulatory molecule such as a cytokine, an interleukin, a chemokine or a ligand or an epitope (such as a helper T cell epitope) and optimally combinations thereof which, when administered with the NOI enhances or potentiates or modulates the CMI response relative to the CMI response generated upon administration of the NOI alone or protein alone. The adjuvant may be any adjuvant known in the art which is appropriate for human or animal use.

Immunomodulatory molecules such as cytokines (TNF-alpha, IL-6, GM-CSF, and IL-2), and co-stimulatory and accessory molecules (B7-1, B7-2) may be used as adjuvants in a variety of combinations. In one embodiment GM-CSF is not administered to subject before, in or after the administration regimen. Simultaneous production of an immunomodulatory molecule and an EOI at the site of expression of the EOI may enhance the generation of specific effectors which may help to enhance the CMI response. The degree of enhancement of the CMI response may be dependent upon the specific immunostimulatory molecules and/or adjuvants used because different immunostimulatory m olecules may elicit different mechanisms for enhancing and/or modulating the CMI response. By way of example, the different effector mechanisms/immunomodulatory molecules include but are not limited to augmentation of help signal (IL-2), recruitment of professional APC (GM-CSF), increase in T cell frequency (IL-2), effect on antigen processing pathway and MHC expression (IFN-gamma and TNF-alpha) and diversion of immune response away from the Th1 response and towards a Th2 response (LTB) (see WO 97/02045). Unmethylated CpG containing oligonucleotides (see WO96/02555) are also preferential inducers of a Th1 response and are suitable for use in the present invention.

Without being bound by theory, the inclusion of an adjuvant is advantageous because the adjuvant may help to enhance the CMI response to the expressed NOI or protein by diverting the Th2 response to a Th1 response and/or specific effector associated mechanisms to an expressed EOI with the consequent generation and maintenance of an enhanced CMI response (see, for example, the teachings in WO 97/02045).

The inclusion of an adjuvant with the NOI or protein is also advantageous because it may result in a lower dose or fewer doses of NOI or protein being necessary to achieve the desired CMI response in the subject to which the NOI or protein is administered, or it may result in a qualitatively and/or quantitatively different immune response in the subject. The effectiveness of an adjuvant can be determined by administering the adjuvant with the NOI or protein in parallel with the NOI or protein alone to animals and comparing antibody and/or cellular-mediated immunity in the two groups using standard assays such as radioimmunoassay, ELISAs, CD8+T-cell assays, and the like, all well known in the art. Typically, the adjuvant is a separate moiety from the antigen, although a single molecule can have both adjuvant and antigen properties.

As used herein, the term “genetic adjuvant” refers to an adjuvant encoded by an NOI and which, when administered with the NOI encoding the EOI or protein (comprising an epitope) enhances the CMI response relative to the CMI response generated upon administration of the NOI or protein alone.

In one preferred embodiment, the genetic adjuvant is a bacterial ADP-ribosylating exotoxin. ADP-ribosylating bacterial toxins are a family of related bacterial exotoxins and include diphtheria toxin (DT), pertussis toxin (PT), cholera toxin (CT), the E. coli heat-labile toxins (LT1 and LT2), Pseudomonas endotoxin A, Pseudomonas exotoxin S, B. cereus exoenzyme, B. sphaericus toxin, C. botulinum C2 and C3 toxins, C. limosum exoenzyme, as well as toxins from C. perfringens, C. spiriforma and C. difficile, Staphylococcus aureus EDIN, and ADP-ribosylating bacterial toxin mutants such as CRM₁₉₇, a non-toxic diphtheria toxin mutant (see, e.g., Bixler et al. (1989) Adv. Exp. Med. Biol. 251:175; and Constantino et al. (1992) Vaccine). Most ADP-ribosylating bacterial toxins are organized as an A:B multimer, wherein the A subunit contains the ADP-ribosyltransferase activity, and the B subunit acts as the binding moiety. Preferred ADP-ribosylating bacterial toxins for use in the compositions of the present invention include cholera toxin and the E. coli heat-labile toxins.

Cholera toxin (CT) and the related E. coli heat labile enterotoxins (LT) are secretion products of their respective enterotoxic bacterial strains that are potent immunogens and exhibit strong toxicity when administered systemically, orally, or mucosally. Both CT and LT are known to provide adjuvant effects for antigen when administered via the intramuscular or oral routes. These adjuvant effects have been observed at doses below that required for toxicity. The two toxins are extremely similar molecules, and are at least about 70-80% homologous at the amino acid level.

Preferably the genetic adjuvant is cholera toxin (CT), enterotoxigenic E. Coli heat-labile toxin (LT), or a derivative, subunit, or fragment of CT or LT which retains adjuvanticity. In an even more preferred embodiment, the genetic adjuvant is LT. In another preferred embodiment, the genetic adjuvant may be CTB or LTB.

Preferably the entertoxin is a non-toxic enterotoxin. By way of example, at least one of the entertoxin subunit coding regions may be genetically modified to detoxify the subunit peptide encoded thereby, for example wherein the truncated A subunit coding region has been genetically modified to disrupt or inactivate ADP-ribosyl transferase activity in the subunit peptide expression product (see WO 03/004055).

In the examples described below, the LT holotoxin comprising native A and B subunits expressed by a plasmid vector was used as a genetic adjuvant which was co-administered with the NOI expressing the HSV-2/HbsAg antigen to obtain an enhanced CMI response. The results demonstrate that the inclusion of an adjuvant greatly improve the capability of eliciting a CMI response, in terms of generating a systemic T cell response compared to administration of an NOI or protein without the adjuvant.

Thus, these results demonstrate that this genetic adjuvant is particularly desirable where an even more enhanced CMI response is desired. Other desirable genetic adjuvants include but are not limited to NOI encoding IL-10, IL-12, IL-13, the interferons (IFNs) (for example, IFN-alpha, IFN-ss, and IFN-gamma), and preferred combinations thereof. Still other such biologically active factors that enhance the CMI response may be readily selected by one of skill in the art, and a suitable plasmid vector containing same constructed by known techniques.

NOI

The EOIs of the present invention may be administered as nucleotide sequences encoding the EOI. As used herein, the term nucleotide sequence of interest (NOI) refers to one of more NOI which encode one or more EOIs which are used in the method of the present invention. The term “nucleotide sequence of interest (NOI)” is synonymous with the term “polynucleotide”. The NOI may be DNA or RNA of genomic or synthetic or of recombinant origin. The NOI may be double-stranded or single-stranded whether representing the sense or antisense strand or combinations thereof. For some applications, preferably, the NOI is DNA. For some applications, preferably, the NOI is prepared by use of recombinant DNA techniques (e.g. recombinant DNA). For some applications, preferably, the NOI is cDNA. For some applications, preferably, the NOI may be the same as the naturally occurring form. The NOI may be isolated or purified form, such as non-cellular form.

Vector

In one embodiment of the present invention, the NOI is administered directly to a host subject. In another embodiment of the present invention, a vector comprising an NOI is administered to a host subject. Preferably the NOI is prepared and/or administered using a genetic vector. As it is well known in the art, a vector is a tool that allows or faciliates the transfer of an entity from one environment to another. In accordance with the present invention, and by way of example, some vectors used in recombinant DNA techniques allow entities, such as a segment of DNA (such as a heterologous DNA segment, such as a heterologous cDNA segment), to be transferred into a host and/or a target cell for the purpose of replicating the vectors comprising the NOI of the present invention and/or expressing the EOIs of the present invention encoded by the NOI. Examples of vectors used in recombinant DNA techniques include but are not limited to plasmids, chromosomes, artificial chromosomes or viruses. The term “vector” includes expression vectors and/or transformation vectors. The term “expression vector” means a construct capable of in vivo or in vitro/ex vivo expression. The term “transformation vector” means a construct capable of being transferred from one species to another.

In one embodiment a viral promoter is used to drive expression from the NOI. The promoter may be a Cytomegalovirus (CMV) promoter. A preferred promoter element (particularly in the case where the NOI encodes an HIV antigen) is the CMV immediate early (IE) promoter devoid of intron A, but including exon 1. Thus the expression from the NOI may be under the control of HCMV IE early promoter.

Naked DNA

The vectors comprising the NOI of the present invention may be administered directly as “a naked nucleic acid construct”, preferably further comprising flanking sequences homologous to the host cell genome. As used herein, the term “naked DNA” refers to a plasmid comprising the NOI of the present invention together with a short promoter region to control its production. It is called “naked” DNA because the plasmids are not carried in any delivery vehicle. When such a DNA plasmid enters a host cell, such as a eukaryotic cell, the proteins it encodes are transcribed and translated within the cell.

Viral Vectors

Alternatively, the vectors comprising the NOI of the present invention may be introduced into suitable host cells using a variety of viral techniques which are known in the art, such 5 as for example infection with recombinant viral vectors such as retroviruses, herpes simplex viruses and adenoviruses. The vector may be a recombinant viral vector. Suitable recombinant viral vectors include but are not limited to adenovirus vectors, adeno-associated viral (AAV) vectors, herpes-virus vectors, a retroviral vector, lentiviral vectors, baculoviral vectors, pox viral vectors or parvovirus vectors (see Kestler et al 1999 Human Gene Ther 10(10):1619-32). In the case of viral vectors, administration of the NOI is mediated by viral infection of a target cell.

Targeted Vector

The term “targeted vector” refers to a vector whose ability to infect or transfect or transduce 15 a cell or to be expressed in a host and/or target cell is restricted to certain cell types within the host subject, usually cells having a common or similar phenotype.

Expression Vector

Preferably, the NOI of the present invention which is inserted into a vector is operably linked to a control sequence that is capable of providing for the expression of the EOI, by the host cell, i.e. the vector is an expression vector. The agent produced by a host cell may be secreted or may be contained intracellularly depending on the NOI and/or the vector used. As will be understood by those of skill in the art, expression vectors containing the NOI can be designed with signal sequences which direct secretion of the EOI through a particular prokaryotic or eukaryotic cell membrane.

Fusion Proteins

The NOI of the present invention may be expressed as a fusion protein comprising an adjuvant and/or a biological response modifier and/or immunomodulator fused to the EOI 30 to further enhance and/or augment the CMI response obtained. The biological response modifier may act as an adjuvant in the sense of providing a generalised stimulation of the CMI response. The EOI may be attached to either the amino or carboxy terminus of the biological response modifier.

Protein Comprising the Epitope

The protein sequence may be the same as the epitope sequence (i.e. without any addition sequence to the N or C terminus). The protein is typically in isolated or purified form, such as non-cellular form. The protein typically as a length of 8 to 400 amino acids, such as 10 to 300 or 15 to 150 amino acids.

NOI and Protien Administration

The NOI or protein may be administered, either alone or as part of a composition, via a variety of different routes. Certain routes may be favoured for certain compositions, as resulting in the generation of a more effective CMI response, or as being less likely to induce side effects, or as being easier for administration. The route of administration for a vaccine composition may vary depending upon the identity of the pathogen or infection to be prevented or treated.

The NOI or protein may be administered via a systemic route or a mucosal route or a transdermal route or it may be administered directly into a specific tissue such as the liver, bone marrow or into the tumour in the case of cancer therapy. As used herein, the term “systemic administration” includes but is not limited to any parenteral routes of administration. In particular, parenteral administration includes but is not limited to subcutaneous, intraperitoneal, intravenous, intraarterial, intramuscular, or intrastemal injection, intravenous, intraarterial, or kidney dialytic infusion techniques. Preferably, the systemic, parenteral administration is intramuscular injection.

In one preferred embodiment of the method, the NOI or protein is administered via the skin, for example by a transdermal route. While it is believed that any accepted mode and route of immunization can be employed and nevertheless achieve some advantages in accordance herewith, the Examples below demonstrate particular advantages with transdermal NOI administration. In this regard, and without being bound by theory, it is believed that transdermal administration is preferred because it more efficiently activates the cell mediated arm of the immune system.

The term “transdermal” delivery intends intradermal (e.g., into the dermis or epidermis), transdermal (e.g.,“percutaneous”) and transmucosal administration, i.e., delivery by passage of an agent into or through skin or mucosal tissue. See, e.g., Transdermal Drug Delivery: Developmental Issues and Research Initiatives, Hadgraft and Guy (eds.), Marcel Dekker, Inc., (1989); Controlled Drug Delivery: Fundamentals and Applications, Robinson and Lee (eds.), Marcel Dekker Inc.,(1987); and Transdermal Delivery of Drugs, Vols. 1-3, Kydonieus and Berner (eds.), CRC Press, (1987). Thus, the term encompasses delivery of an agent using a particle delivery device (e.g., a needleless syringe) such as those described in U.S. Pat. No. 5,630,796, as well as delivery using particle-mediated delivery devices such as those described in U.S. Pat. No. 5,865,796.

As used herein, the term “mucosal administration” includes but is not limited to oral, intranasal, intravaginal, intrarectal, intratracheal, intestinal and ophthalmic administration.

Mucosal routes, particularly intranasal, intratracheal, and ophthalmic are preferred for protection against natural exposure to environmental pathogens such as RSV, flu virus and cold viruses or to allergens such as grass and ragweed pollens and house dust mites. The enhancement of the CMI response will enhance the protective effect against a subsequently encountered target antigen such as an allergen or microbial agent.

In one embodiment of the method of the invention all of the administrations of the first and/or second and/or a subsequent immunisation are to sites which drain to the same lymph node.

In another preferred embodiment of the present invention, the NOI or protein may be administered to cells which have been isolated from the host subject. In this preferred embodiment, preferably the NOI encoding an EOI from a tumour associated antigen (TAA) is administered to professional antigen presenting cells (APCs), such as dendritic cells.

APCs may be derived from a host subject and modified ex vivo to express an EOI and then transferred back into the host subject to induce an enhanced CMI response against the TAA in order to elicit an anti-tumour response. Dendritic cells are believed to be the most potent APCs for stimulating enhanced CMI responses because the expressed EOIs must be acquired, processed and presented by professional APCs to T cells (both Th1 and Th2 helper cells as well as CD8+T-cells) in order to induce an enhanced CMI response. In an alternative embodiment, cancer cells from a host subject may be modified in situ or in vitro.

NOI Particale Administration

Particle-mediated methods for delivering NOI or protein preparations are known in the art. Thus, once prepared and suitably purified, the above-described NOIs can be coated onto core carrier particles using a variety of techniques known in the art. Carrier particles are selected from materials which have a suitable density in the range of particle sizes typically used for intracellular delivery from a gene gun device. The optimum carrier particle size will, of course, depend on the diameter of the target cells.

By “core carrier” is meant a carrier on which a guest nucleic acid (e.g., DNA, RNA) is coated in order to impart a defined particle size as well as a sufficiently high density to achieve the momentum required for cell membrane penetration, such that the guest molecule can be delivered using particle-mediated techniques (see, e.g., U.S. Pat. No. 5,100,792). Core carriers typically include materials such as tungsten, gold, platinum, ferrite, polystyrene and latex. See e.g., Particle Bombardment Technology for Gene Transfer, (1994) Yang, N. ed., Oxford University Press, New York, N.Y. pages 10-11. Tungsten and gold particles are preferred. Tungsten particles are readily available in average sizes of 0.5 to 2.0 microns in diameter. Gold particles or microcrystalline gold (e.g., gold powder A1570, available from Engelhard Corp., East Newark, N.J.) will also find use with the present invention. Gold particles provide uniformity in size (available from Alpha Chemicals in particle sizes of 1-3 microns, or available from Degussa, South Plainfield, N.J. in a range of particle sizes including 0.95 microns). Microcrystalline gold provides a diverse particle size distribution, typically in the range of 0.5-5 microns.

However, the irregular surface area of microcrystalline gold provides for highly efficient coating with nucleic acids.

A number of methods are known and have been described for coating or precipitating NOIs or proteins onto gold or tungsten particles. Most such methods generally combine a predetermined amount of gold or tungsten with plasmid DNA, CaCl₂ and spermidine. The resulting solution is vortexed continually during the coating procedure to ensure uniformity of the reaction mixture. After precipitation of the NOI, the coated particles can be transferred to suitable membranes and allowed to dry prior to use, coated onto surfaces of a sample module or cassette, or loaded into a delivery cassette for use in particular gene gun instruments.

By “particle delivery device” is meant an instrument which delivers a particulate composition transdermally without the aid of a conventional needle to pierce the skin. Particle delivery devices for use with the present invention are discussed throughout this document. Various particle acceleration devices suitable for particle-mediated delivery are known in the art, and are all suited for use in the practice of the invention. Current device designs employ an explosive, electric or gaseous discharge to propel the coated carrier particles toward target cells. The coated carrier particles can themselves be releasably attached to a movable carrier sheet, or removably attached to a surface along which a gas stream passes, lifting the particles from the surface and accelerating them toward the target. An example of a gaseous discharge device is described in U.S. Pat. No. 5,204,253. An explosive-type device is described in U.S. Pat. No. 4,945,050. One example of a helium discharge-type particle acceleration apparatus is the PowderJect XR instrument (PowderJect Vaccines, Inc., Madison), Wis., which instrument is described in U.S. Pat. No. 5,120,657. An electric discharge apparatus suitable for use herein is described in U.S. Pat. No. 5,149,655. The disclosure of all of these patents is incorporated herein by reference.

Alternatively, particulate NOI or protein compositions can administered transdermally using a needleless syringe device. For example, a particulate composition comprising the NOIs of the present invention can be obtained using general pharmaceutical methods such as simple evaporation (crystallization), vacuum drying, spray drying or lyophilization. If desired, the particles can be further densified using the techniques described in commonly owned International Publication No. WO 97/48485, incorporated herein by reference. These particulate compositions can then be delivered from a needleless syringe system such as those described in International Publication Nos. WO 94/24263, WO 96/04947, WO 96/12513, and WO 96/20022, all of which are incorporated herein by reference. Delivery of particles comprising antigens or allergens from the above referenced needleless syringe systems is practiced with particles having an approximate size generally ranging from 0.1 to 250 microns, preferably ranging from about 10-70 microns. Particles larger than about 250 microns can also be delivered from the devices, with the upper limitation being the point at which the size of the particles would cause untoward damage to the skin cells. The actual distance which the delivered particles will penetrate a target surface depends upon particle size (e. g., the nominal particle diameter assuming a roughly spherical particle geometry), particle density, the initial velocity at which the particle impacts the surface, and the density and kinematic viscosity of the targeted skin tissue. In this regard, optimal particle densities for use in needleless injection generally range between about 0.1 and 25 g/cm3, preferably between about 0.9 and 1.5 g/cm3, and injection velocities generally range between about 100 and 3,000 m/sec. With appropriate gas pressure, particles having an average diameter of 10-70 Rm can be accelerated through the nozzle at velocities approaching the supersonic speeds of a driving gas flow.

The particle compositions or coated particles are administered to the individual in a manner compatible with the dosage formulation, and in an amount that will be effective for the purposes of the invention. The amount of the composition to be delivered (e. g., about 0.1 mg to 1 mg, more preferably 1 to 50 ug of the antigen or allergen, depends on the individual to be tested. The exact amount necessary will vary depending on the age and general condition of the individual to be treated, and an appropriate effective amount can be readily determined by one of skill in the art upon reading the instant specification.

Gold or tungsten microparticles can also be used as transporting agents, as described in WO 93/17706, and Tang petal., Nature (1992)356:152. In this particular case, the NOI is precipitated on the microparticles in the presence of calcium chloride and spermidine, and then the whole is administered by a high-speed jet into the dermis or into the epidermis using an apparatus with no needle, such as those described in U.S. Pat. Nos. 4,945,050 and 5,015,580, and WO 94/24243. The quantity of NOI that can be used to vaccinate a host subject depends on a number of factors such as, for example, the strength of the promoter used to express the antigen, the immunogenicity of the product expressed, the condition of the mammal for whom the administration is intended (e.g., the weight, age, and general state of health), the mode of administration, and the type of formulation. In general, an appropriate dose for prophylactic or therapeutic use in an adult of the human species is from about 1 pg to about 5 mg, preferably from about 10 pg to about 1 mg, most preferably from about 25 pg to about 500 pg. Particle-mediated delivery techniques have been compared to other types of NOI administration, and found markedly superior. Fynan et al. (1995) Int. J. Immunopharmacology 17:79-83, Fynan et al. (1993) Proc. Natl. Acad. Sci. USA 90:11478-11482, and Raz et al. (1994) Proc. Natl. Acad. Sci. USA 91:9519-9523. Such studies have investigated particle-mediated delivery of nucleic acid-based vaccines to both superficial skin and muscle tissue. One possible reason for the markedly better results achieved with the gene gun is that the NOI is delivered intracellularly as opposed to the extracellular delivery by intramuscular injection.

Preferably the interval between administration of the target antigen (TA) ranges from about 48 hours to about 192 hours. More preferably, the interval between administration of the target antigen (TA) ranges from about 72 hours to about 168 hours. Even more preferably, the intervals between administration of the target antigen (TA) ranges from about 72 hours to about 144 hours.

Host Mammalian Subject

As used herein, the term “host mammalian subject” means any member of the subphylum cordata, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. Preferred animals include those used in sports, such as racehorses or show jumping horses.

The terms do not denote a particular age. Thus, both adult and newborn individuals are intended to be covered. The method described herein is intended for use in any of the above vertebrate species, since the immune systems of all of these vertebrates operate similarly. If a mammal, the subject will preferably be a human, but may also be a domestic livestock, laboratory subject or pet animal.

Prevent and/or Treat

This method or process of the present invention is broadly applicable to vaccination methods and is relevant to the development of prophylactic and/or therapeutic vaccines (including immunotherapeutic vaccines). It is to be appreciated that all references herein to treatment include curative, palliative and prophylactic treatment.

In the method of the present invention, the NOI or protein described herein may be employed alone as part of a composition, such as but not limited to a pharmaceutical composition or a vaccine composition or an immunotherapeutic composition to prevent and/or treat a T cell mediated immune disorder. The administration of the NOI or protein or a composition comprising the NOI or protein may be for either “prophylactic” or “therapeutic” purpose. As used herein, the term “therapeutic” or “treatment” includes any of following: the prevention of infection or reinfection; the reduction or elimination of symptoms; and the reduction or complete elimination of a pathogen. Treatment may be effected prophylactically (prior to infection) or therapeutically (following infection).

Prophylaxis or therapy includes but is not limited to eliciting an effective CMI immune response to an NOI and/or alleviating, reducing, curing or at least partially arresting symptoms and/or complications resulting from a T cell mediated immune disorder. When provided prophylactically, the composition of the present invention is typically provided in advance of any symptom. The prophylactic administration of the NOI or composition of the present invention is to prevent or ameliorate any subsequent infection or disease. When provided therapeutically, the NOI or composition of the present invention is typically provided at (or shortly after) the onset of a symptom of infection or disease. Thus the composition of the present invention may be provided either prior to the anticipated exposure to a disease causing agent or disease state or after the initiation of an infection or disease.

Whether prophylactic or therapeutic NOI or protein administration (either alone or as part of a composition) is the more appropriate will usually depend upon the nature of the disease. By way of example, the immunotherapeutic composition of the present invention could be used in immunotherapy protocols to actively inducing tumour immunity by vaccination with a tumour cell or its antigenic components. This latter form of treatment is advantageous because the immunity is prolonged and because there is a general belief that one of the best ways to eliminate tumours would be to induce a strong specific anti-tumour CTL response. On the other hand a vaccine composition will preferably, though not necessarily be used prophylactically to induce an effective CMI response against subsequently encountered antigens or portions thereof (such as epitopes) related to the target antigen.

Prophylactically or Therapeuticaly Effective Amount

The dose of NOI or protein administrated to a host subject, in the context of the present invention, should be sufficient to effect a beneficial prophylactic or therapeutic CMI response in the subject over time.

As used herein, the term “prophylactically or therapeutically effective dose” means a dose in an amount sufficient to elicit an enhanced CMI response to one or more EOIs of a specific target antigens and/or to alleviate, reduce, cure or at least partially arrest symptoms and/or complications from a disease, such as a T cell mediated immune disorder

Dosage

Prophylaxis or therapy can be accomplished by a single direct administration at a single time point or multiple time points. Administration can also be delivered to a single or to multiple sites. Some routes of administration, such as mucosal administration via ophthalmic drops may require a higher dose. Those skilled in the art can adjust the dosage and concentration to suit the particular route of delivery. Advantageously, the Examples demonstrate that a single dose of the NOI or the composition comprising the NOI is usually sufficient to achieve an enhanced CMI response.

Conditions and Diseases

Because the method of the invention elicits an enhanced CMI response, the method can be used to protect against subsequent infection by a pathogen such as a viral, bacterial, parasitic or other infectious agent. Preferably the target antigen is a pathogen or an antigen associated with an infectious disease, an allergen or a cancer. Examples of infectious disease include but are not limited to viral, bacterial, mycobacterial and parasitic disease. Examples of allergens include, but are not limited to, plant pollens, dust mite proteins, animal dander, saliva and fungal spores. Examples of tumour-associated antigens (TAAs) include, but are not limited to, live or irradiated tumor cells, tumor cell extracts and protein subunits of tumour antigens. The antigen can also be a sperm protein for use in contraception. In some embodiments, the antigen is an environmental antigen. Examples of environmental antigens include, but are not limited to, respiratory syncytial virus (“RSV′), flu viruses and cold viruses. Pathogens which invade via the mucosa also include those that cause respiratory syncytial virus, flu, other upper respiratory conditions, as well as agents which cause intestinal infections.

Amongst several known examples of other diseases against which an enhanced CMI response is important are the following: infection and disease caused by viruses such as but not limited to HIV, herpes simplex, herpes zoster, hepatitis C, hepatitis B, influenza, Epstein-Barr virus, measles, dengue, HTLV-1 and human papilloma virus (HPV) (for example HPV 16); diseases caused by bacteria such as but not limited to Mycobacterium tuberculosis and Listeria sp, Chlamydia, Mycobacteria, Plasmodium Falciparum, Legioniella and enteropathogenic, enterotoxigenic, enteroinvasive, enterohaemorrhagic and enteroaggregative E.coli and and diseases caused by Pathogenic protozoans which include but which are not limited to malaria, Babesia, Schistosoma, Toxiplasma and Toxocara canis or by the protozoan parasites Toxoplasma and Trypanosoma. Furthermore, the administration regime described herein is expected to be of value in immunising against forms of cancer where T cell responses plays a protective role. Examples of cancers of mammals which may be treated using method and compositions of the present invention include but are not limited to melanoma, metastases, adenocarcinoma, thymoma, lymphoma, sarcoma, lung cancer, liver cancer, colon cancer, non-Hodgkins lymphoma, Hodgkins lymphoma, leukemias, uterine cancer, breast cancer, prostate cancer, ovarian cancer, cervical cancer, bladder cancer, kidney cancer, pancreatic cancer and the like. In one embodiment the cancer is one which is linked to, for example caused by, HPV (e.g. HPV 16). Such a cancer may be a cervical cancer.

Cancer

In one preferred embodiment the use of an NOI encoding an EOI (or a protein comprising an epitope) from a tumour associated target antigen (TAA) in the method of the present invention allows for the development of targeted antigen-specific vaccines for cancer therapy. The administration of an NOI encoding an EOI from a TAA and optionally an NOI encoding an immunomodulatory molecule provides a powerful system to elicit a specifically enhanced CMI response in terms of prevention in a host subject with an increased risk of cancer development (preventive immunisation), prevention of disease recurrence after primary surgery (anti-metastatic vaccination), or as a tool to expand the number of T cells in vivo, thus improving their effectiveness in eradication of diff-use tumours (treatment of established disease). Furthermore, the method of the present invention may be used to elicit an enhanced CMI response in a host subject by the treatment of cells ex vivo prior to being transferred back to the tumour bearer (also known as adoptive immunotherapy). The method or process of the present invention can be used to administer the NOI into the host subject either prior to any evidence of cancers such as melanoma (=preventative vaccination) or to mediate regression of the disease in a mammal afflicted with a cancer such as melanoma (therapeutic or immunotherapeutic vaccination).

In one embodiment the NOI encodes an antigen from HPV (for example HPV 16), such as E6 or E7.

Activated T-Cells

In other aspects, the present invention relates to a method of preparing activated T-cells. In its most general sense, this method includes administering, preferably transdermally, a NOI encoding a preselected EOI of a target antigen capable of enhancing a CMI response in terms of an enhanced T-cell response, to a host subject. In accordance with this aspect of the invention, the T-cells are recovered from lymph nodes of the host for further use.

Numerous potential uses of specifically activated T-cells are envisioned. For example, in the case of human therapy, it is contemplated that specifically activated T-cells may be cultured ex vivo and administered to humans for the treatment of viral infections or patients with cancer. In accordance with this aspect of the invention, the T-cells are prepared by administering the NOI in vivo and then isolating the T-cells to expand in vitro in the presence of appropriate biological response modifiers and/or immunomodulators and/or adjuvants such as but not limited to peptide, cytokines and antigen presenting cells.

Homologues

Proteins (including protein antigens), such as Gag, nef and/or RT, as used in the invention (as encoded by the NOI) may have homology and/or sequence identity with naturally occurring forms. Similarly NOI coding sequences capable of expressing such proteins will generally have homology and/or sequence identity with naturally occurring sequences. Techniques for determining nucleic acid and amino acid “sequence identity” also are known in the art. Typically, such techniques include determining the nucleotide sequence of the MRNA for a gene and/or determining the amino acid sequence encoded thereby, and comparing these sequences to a second nucleotide or amino acid sequence.

In general, “identity” refers to an exact nucleotide-to-nucleotide or amino acid-to-amino acid correspondence of two polynucleotides or polypeptide sequences, respectively. Two or more sequences (polynucleotide or amino acid) can be compared by determining their “percent identity.” The percent identity of two sequences, whether nucleic acid or amino acid sequences, is the number of exact matches between two aligned sequences divided by the length of the shorter sequences and multiplied by 100.

An approximate alignment for nucleic acid sequences is provided by the local homology algorithm of Smith and Waterman, Advances in Applied Mathematics 2: 482-489 (1981). This algorithm can be applied to amino acid sequences by using the scoring matrix developed by Dayhoff, Atlas of Protein Sequences and Structure, M. O. Dayhoff ed., 5 suppl. 3: 353-358, National Biomedical ResearchFoundation, Washington, D.C., U.S.A., and normalized by Gribskov, Nucl. AcidsRes. 14 (6): 6745-6763 (1986). An exemplary implementation of this algorithm to determine percent identity of a sequence is provided by the Genetics Computer Group (Madison, Wis.) in the “BestFit” utility application. The default parameters for this method are described in the Wisconsin Sequence Analysis Package Program Manual, Version 8 (1995) (available from Genetics Computer Group, Madison, Wis.). A preferred method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, Calif.).

From this suite of packages the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found at the following internet address: http://www.ncbi.nlm. gov/cgi-bin/BLAST.

Alternatively, homology can be determined by hybridization of polynucleotides under conditions which form stable duplexes between homologous regions, followed by digestion with single-stranded-specific nuclease (s), and size determination of the digested fragments. Two DNA, or two polypeptide sequences are “substantially homologous” to each other when the sequences exhibit at least about 80%-85%, preferably at least about 90%, and most preferably at least about 95%-98% sequence identity over a defined length of the molecules, as determined using the methods above.

As used herein, substantially homologous or homologous also refers to sequences showing complete identity to the specified DNA or polypeptide sequence. DNA sequences that are substantially homologous or homologous can be identified in a Southern hybridization experiment under, for example, stringent conditions, as defined for that particular system. For example, stringent hybridization conditions can include 50% formamide, 5× Denhardt's Solution, 5×SSC, 0.1% SDS and 100 pg/ml denatured salmon sperm DNA and the washing conditions can include 2×SSC, 0.1% SDS at 37 C followed by lx SSC, 0.1% SDS at 68 C. Defining appropriate hybridization conditions is within the skill of the art.

Assays Methods

The effectiveness of the method of the invention may be tested by the steps of (i) administering a NOI as described herein to a host subject such as a human subject or an experimental animal such as a mouse, rat, rabbit, guinea pig, goat, rhesus monkey, or chimpanzee; (ii) thereafter collecting cells from blood, spleen or other lymphoid tissue from the host subject, and (iii) testing the tissue for the presence of activated T-cells, such as T cells that are primed to kill or lyse cells producing a component of an infectious agent.

Once NOI administration has been effected, the T-cells from the lymphoid tissue of the host subject are recovered. The preferred lymphoid tissue will be lymph node tissue, and most preferably tissue from draining lymph nodes proximal to the site of NOI administration. As used herein, the word “proximal node” is intended to refer to the node or nodes that are located proximal to the site of NOI administration.

Such nodes are physically located in the proximity of the site of NOI administration or in the area draining the site of administration, and also included are those draining nodes that are physically at a greater distance from the administration site.

The final step of the assay method involves determining whether the T-cells have been activated. Typically, the level of T-cell activation can be measured by assays which include but are not limited to radioactive chromium-release assays, or other radioisotope assays, or single cell assays. In addition, single cell T-cell assays using vital stains and/or cell sorters may be employed. A preferred method for measurement of activation of T-cells involves contacting a killing effective amount of the T-cells with MHC-matched target cells that exhibit the candidate EOI on their cell surfaces; maintaining the contact for a time period sufficient for the T-cells to lyse the target cells; and determining the degree of T-cell mediated lysis of the target cells. However, any method capable of detecting a specific T-cell response may be employed, including but not limited to chromium release assays, single-cell assays or even determination of cell-cell conjugates.

In one embodiment the invention provides an assay for testing the effectiveness of a method of eliciting a T cell response, wherein the method of eliciting a T cell response is the same as any such method described herein. Thus the method may comprise (i) a first immunisation that comprises at least two administrations which are from 1 to 14 days apart to the subject, wherein each administration comprises administering a nucleotide of interest (NOI) encoding a T cell epitope, and optionally (ii) a second immunisation that comprises at least one administration to the subject of (a) a NOI encoding the T cell epitope, or (b) a protein comprising the T cell epitope, wherein the time between
the first administration of the first immunisation, and
the first administration of the second immunisation, is from 21 to 365 days,

wherein the assay comprises carrying out the method on a mammalian subject and then determining the level of activated or memory T cells specific to the epitope in the subject.

It is believed that a higher level of T cell response is achieved if (i) all of the administrations of the first immunisation occur before the level of activated T cells which are generated by the initial administration(s) return to a basal level, and (b) the second immunisation occurs after the return of T cells to a basal level. The assay described above may therefore be used to determine whether or not a given method of eliciting a T cell response has a first and second immunisation which are at appropriate times relative to the levels of activated T cells. In one embodiment the assay comprises determining whether (i) the administrations of the first immunisation all fall within the time period between the first administration of the first immunisation and the decline in the level of activated T cells to basal level, and/or(ii) the first administration of the second immunisation occurs after the decline in the level of activated T cells to basal level.

Other Aspects

In a further aspect, there is provided a method of selectively eliciting an enhanced humoral response without necessarily eliciting an associated enhancement of the CMI response wherein the method comprises administration of an NOI encoding one or more EOIs of a TA at least three times to the host subject wherein the time interval between each NOI administration is about 48 hours and wherein the method is effective to provide an enhanced humoral immune response against the or each expressed EOI in the host mammalian subject.

In another aspect, there is provide a method of eliciting an enhanced humoral response wherein the method comprises administration of an NOI encoding one or more EOIs of a TA at least three times to the host subject wherein the time interval between each NOI administration is about 28 days and wherein the method is effective to provide an enhanced CMI response that helps to enhance the humoral immune response against the or each expressed EOI in the host mammalian subject.

Compositions

The present invention provides compositions that are useful for preventing and/or treating T cell mediated immune disorders. In one embodiment, the composition is a pharmaceutical composition. In another preferred embodiment, the composition is an immunotherapeutic composition. In an even more preferred embodiment, the composition is a vaccine composition. The composition can comprise a therapeutically or prophylactically effective amount of an NOI encoding an EOI of a TA of the invention as described above. The composition may also comprise a carrier such as a pharmaceutically or immunologically acceptable carrier. Pharmaceutically acceptable carriers or immunologically acceptable carriers are determined in part by the particular composition being administered as well as by the particular method used to administer the composition. Accordingly, there is a wide variety of suitable formulations of pharmaceutical compositions or vaccine compositions or immunotherapeutic compositions of the present invention.

Formulations

The NOI or protein may be formulated into a pharmaceutical composition or an immunotherapeutic composition or a vaccine composition. Such formulations comprise the NOI or protein combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampoules or in multi-dose containers containing a preservative. Formulations include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (for eg, a powder or granules) form for reconstitution with a suitable vehicle (e. g., sterile pyrogen-free water) prior to parenteral administration of the reconstituted composition. The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono-or di-glycerides.

Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Also included in the invention is a kit for enhancing a CMI response to an EOI of a target antigen. Such a kit comprises an NOI encoding an EOI of a TA and/or a protein comprising the epitope. The kit may also include an adjuvant, preferably a genetic adjuvant is administered with or as part of the NOI or protein and instructions for administering the NOI or protein. Other preferred components of the kit include an applicator for administering the NOI or protein. As used herein, the term “applicator” refers to any device including but not limited to a hypodermic syringe, gene gun, particle acceleration device, nebulizer, dropper, bronchoscope, suppository, impregnated or coated vaginally-insertable material such as a tampon, douche preparation, solution for vaginal irrigation, retention enema preparation, suppository, or solution for rectal or colonic irrigation for applying the NOI either systemically or mucosally or transdermally to the host subject.

### EXAMPLES

The following invention will now be further described only by way of example in which reference is made to the following Figures. The following examples are presented only to illustrate the present invention and to assist one of ordinary skill in making and using the same. The examples are not intended in any way to otherwise limit the scope of the invention. Efforts have been made to ensure accuracy with respect to numbers used (e.g., amounts, temperatures, etc.), but some experimental error and deviation should, of course, be allowed for.

### BRIEF DESCRIPTION OF THE FIGURES

FIG. 1 provides (CD8+T cell) responses after 1, 2 or 4 NOI administrations to mice during 1 week at day 7 (D 7), days 0 and 7 (D 0,7) or days 0, 2, 4, and 7 (D 0,2,4,7) respectively. Either 1 or 2 shots per NOI administration were given, and in one group LT adjuvant was co-administered with the NOI.

FIG. 1A demonstrates the CD8+T cell response after administration of the ICP27 single gene plasmid.

FIG. 1B demonstrates the CD8+T cell response after administration of the PJV7630 multi- gene plasmid.

FIG. 2 demonstrates protection of mice from infectious challenge using clustered NOI administration.

FIG. 3A demonstrates optimal time intervals between clustered administrations Of the ICP27 single gene plasmid.

FIG. 3B demonstrates cellular responses obtained after clustered NOI administration of HbsAg single gene plasmid.

FIG. 3C demonstrates antibody titre obtained after clustered NOI administration of HbsAg single gene plasmid.

FIG. 4A demonstrates ELISPOT measurements 1 week post administration of clustered NOI administrations of a multigene plasmid (PJV7630) at intervals of 0, 1, 2, 4 and 6 days between administrations

FIG. 4B demonstrates ELISPOT measurements 3 weeks post administration of clustered NOI administrations of a multigene plasmid (PJV7630) at intervals of 0, 1, 2, 4 and 6 days between administrations.

FIG. 5A is a schematic diagram showing that each “administration” is made up of 1 to 4 XRI administrations and the resting period between each administration.

FIG. 5B shows the CMI response in terms of IFN-gamma release obtained from a combination of genetic LT adjuvant and a clustered NOI administration schedule.

FIG. 5C shows the antibody response obtained from a combination of genetic LT adjuvant and a clustered NOI administration schedule.

FIG. 6A shows IFN-γ ELISPOT data obtained from domestic pigs following the first cluster immunization.

FIG. 6B shows the average area of erythema present at the site of antigen administration in pigs (4 animals) immunized with pPJV7630.

FIG. 7 shows an anti-HA antibody response in domestic pigs.

FIG. 8 shows plasmid pPJV1671 which is a human DNA vaccine vector encoding the hemagglutinin (HA) antigen of influenza A/Panama/2007/99 (H3N2).

FIG. 9 shows a comparison of H3 Panama HA Natural Sequence with a H3 Panama HA Encoded by pPJV1671 and a Consensus sequence.

FIG. 10 shows a plasmid map of pPJV2012.

FIG. 11 shows a plasmid map for pPJV7563.

FIG. 12 provides a nucleotide sequence for the pPJV7563 plasmid.

FIG. 13 provides a flowchart outlining the construction of PJV7563.

FIG. 14 (i) to (viii) provides the Feature Maps of Key Plasmids in the Construction of pPJV7563.

FIG. 15 provides a Flowchart Derivation of Plasmids WRG7074 and WRG7128.

FIG. 16 (i) to (v) provides Further Key Plasmid Feature Maps

FIGS. 17 to 22 relate to constructs that express HIV antigens and to sequences that encode HIV antigens.

FIGS. 23 to 28 relate to immunization with HPV E6 and E7 antigens.

FIGS. 29 to 38 show results from firther experiments which investigate the response obtained by use of the method of the invention.

### GENERAL TECHNIQUES

Unless otherwise indicated, the recombinant DNA techniques utilized in the present invention are standard procedures, well known to those skilled in the art. Such techniques are described and explained throughout the literature in sources such as, J. Perbal, A Practical Guide to Molecular Cloning, John Wiley and Sons (1984); J. Sambrook et al., Molecular Cloning:A Laboratory Manual, Cold Spring Harbour Laboratory Press (1989); T. A.Brown (editor), Essential Molecular Biology: A Practical Approach, Volumes 1 and 2, IRL Press (1991); D. M. Glover and B. D. Hames (editors), DNA Cloning: A Practical Approach, Volumes 1-4, IRL Press (1995 and 1996); and F. M. Ausubel et al. (editors), Current Protocols in Molecular Biology, GreenePub. Associates and Wiley-Interscience (1988, including all updates until present) and are incorporated herein by reference. The method of the present invention involves the direct in vivo introduction of an NOI encoding at least one or more EOI of a TA into tissues of a subject for expression of the EOI by the cells of the subject's tissue.

The NOI constructs of the present invention may be prepared by conventional methods known to one of skill in the art. Methods for constructing of the DNA plasmid or recombinant vectors are described in conventional texts, such as Burger et al., J. Gen. Virol., 72: 359-367 (1991), and are well-known in the art. See also Sambrook et al., 1989, Molecular Cloning: A Laboratory Manual, Cold Spring Harbor Laboratory Press, New York; and Ausubel et al., 1997, Current Protocols in Molecular Biology, Green & Wiley, New York.

By way of example, NOIs which encode one or more EOIs of the TA or sequences sufficiently homologous to known EOIs of TA to induce CMI responses may be obtained by following well known procedures described in the art for the isolation of NOIs from a variety of microorganism sources. Alternatively, the NOIs encoding the EOIs of the TA may be synthesized in a nucleic acid synthesizer. Thus, the invention includes synthetic forms of NOIs encoding EOIs of the TA. Other recombinant bacterial plasmids or viral vectors which contain such isolated NOIs and which are preferably capable of directing expression of the EOI of the TA encoded by the NOI in a host cell; and cells containing such vectors, either eukaryotic or prokaryotic cells, preferably eukaryotic cells are also prepared by known techniques. To ensure expression of the EOI of the TA by the NOI in plasmid or viral vector form, the NOI is operably linked to a promoter/regulatory region capable of driving high levels of expression of the antigen in the host cells. Many such promoter/regulatory sequences are available in the art including, but not limited to, for example, the human cytomegalovirus immediate early promoter/enhancer sequence, the SV40 early promoter, the Rous sarcoma virus promoter and other mammalian promoter/enhancer sequences. As used herein, the term “promoter/regulatory sequence” refers to a DNA sequence which is required for expression of an NOI operably linked to the promoter/regulatory sequence. In some instances, the promoter/regulatory sequence may function in a tissue specific manner, in that, the promoter/regulatory sequence is only capable of driving expression in a cell of a particular tissue type. Unless otherwise indicated, selection of any particular plasmid vector or other DNA vector or viral vector is not a limiting factor in this invention and other DNA or viral vectors may be substituted for those disclosed herein upon a reading of the present disclosure. It is well within the skill of the artisan to choose particular promoter/regulatory sequences and operably link those 5 promoter/regulatory sequences to a DNA sequence encoding a desired antigen. Such technology is well known in the art and is described, for example, in Sambrook (ibid), and Ausubel (ibid).

The following general methods were used to carry out the studies described in Examples 1-5 below. In each study, NOIs comprising EOIs were coated onto gold particles in order to provide exemplary compositions according the present invention. The coated particles were administered to animal subjects, and the ability of the compositions to elicit antigen specific T cell and/or antibody responses was assessed.

Core Carrier Particle Coating

Appropriate weights of gold particles were weighed directly into 1.5 mL Eppendorf tubes.

Approximately 300 μL of a 0.05M spermidine solution was then added to suspend the gold, using a sonicator to disperse the gold. A solution (approximately 50 μL) containing the relevant DNA plasmid was then added to the gold/spermidine solution at a concentration of 2 μg DNA/mg gold. The DNA solution may contain one type of plasmid, or for certain experiments two or more plasmids (a genetic adjuvant for example) can be mixed together prior to the mixing with the gold solution. The DNA/gold mixture was vortexed at a gentle speed and 300 μL of a 10% CaCl2 solution was added drop-wise while vortexing. The DNA/gold particles were allowed to precipitate at room temperature and then centrifuged briefly (10-15 seconds) to pellet the gold. The pellet was washed three times with approximately 800 SLL of EtOH. The DNA/gold particles were then suspended in a 0.03 mg/mL PVP (polyvinylpyrrolidone) solution made up in EtOH at approximately 1 mg DNA/gold in 3 mL of PVP solution. This solution was then coated onto Tefzel tubing as previously described. See e.g., PCT patent application PCT/US95/00780 and U.S. Pat. Nos. 5,733,600; 5,780,100; 5,865,796 and 5,584,807 the disclosures of which are hereby incorporated by reference.

Hepititis B Surface Antigen (HBSAG) Construction of Plasmid (PWRG7128 Construct

A Hepatitis B surface antigen (HBsAg) vector plasmid was constructed as follows. To generate the HbsAg coding region, the pAM6 construct (obtained from the American Type Culture Collection “ATCC”) was cut with NcoI and treated with mung bean nuclease to remove the start codon of the X-antigen. The resultant DNA was then cut with BamHI and treated with T4 DNA polymerase to blunt-end the DNA and creates an HBsAg expression cassette. The HBsAg expression cassette is present in the 1.2 kB fragment. The plasmid construct pPJV7077 (Schmaljohn et al. (1997) J. Virol. 71:9563-9569) which contains the full-length human CMV (Towne strain) immediate early promoter (with enhancer) was cut with HindIII and BglII, and then treated with T4 DNA polymerase and calf-alkaline phosphatase to create blunt-ended DNA, and the HBsAg expression cassette was ligated into the plasmid to yield the pWRG7128 construct.

In Vitro Immune Assays

Serum samples of individual mice were tested for antibodies specific for HBsAg using an ELISA assay. For the ELISA, Falcon Pro Bind microtiter plates were coated overnight at 4° C. with purified HBsAg (BioDesign) at 0.1 μg per well in PBS (phosphate buffered saline, BioWhittaker). The plates were blocked for 1 hour at room temperature (RT) with 5% dry milk/PBS then washed 3 times with wash buffer (10 mM Tris Buffered saline, 0.1% Brij-35), and serum samples diluted in dilution buffer (2% dry milk/PBS/0.05% Tween 20) were added to the plate and incubated for 2 hours at RT. The plates were washed 3 times and a biotinylated goat anti-mouse antibody (Southern Biotechnology) diluted 1:8000 in dilution buffer was added to the plate and incubated for 1 hr at RT. Following the incubation, plates were washed 3 times, after which a Streptavidin-Horseradish peroxidase conjugate (Southern Biotechnology) diluted 1:8000 in PBS was added and the plate incubated a further 1 hr at RT. After an additional three washes, Plates were washed 3 times, then a TMB substrate solution (13ioRad) was added and the reaction was stopped with IN H₂SO₄ after 30 minutes. Optical density was read at 450 nm. Endpoint titers were calculated by comparison of the samples with a standard of known titer.

For the cellular immune assays, single cell suspensions of splenocytes from the spleens of the immunized animals were cultured in vitro in the presence of a peptide corresponding to a known CD8 epitope in Balb/c mice. The peptide was dissolved in DMSO (10 mg/ml) and diluted to 10 ug/ml in culture. The sequence of the peptide was IPQSLDSWWTSL (SEQ ID NO: 20).

For IFN-γ ELISPOT assays, Millipore Multiscreen membrane filtration plates were coated with 50 μl of 15 μg/ml anti-IFN-γ antiserum (Pharmingen) in sterile 0.1 M carbonate buffer (pH 9.6) overnight at 4° C. Plates were washed 6 times with sterile PBS and then blocked with tissue culture medium containing 10% fetal bovine serum (FBS) for 1-2 hr at RT. The medium was removed and spleen cells dispensed into the wells with a total of 1×10⁶ cells per well. For wells in which less than 1×10⁶ cells from immunized animals was added, cells from naïve animals were used to bring the total to 1×10⁶. Cells were incubated overnight in a tissue culture incubator in the presence of the peptide as described above. The plates were then washed 2 times with PBS and 1 time with distilled water. This was followed by 3 washes with PBS. A biotinylated anti IFN-γ monoclonal antibody (Pharmingen) was added to the plate (50 μl of a 1 μg/ml solution in PBS) and incubated for 2 hr at RT. The plates were washed 6 times with PBS after which 50 μl of a Streptavidin Alkaline phosphatase conjugate (1:1000 in PBS, Pharmingen) was added and incubated for 2 hr at RT. The plates were washed 6 times with PBS and an alkaline phosphatase color substrate (BioRad) was added and the reaction was allowed to proceed until dark spots appeared. The reaction was stopped by washing with water 3 times. Plates were air dried and spots counted under a microscope.

HIV-1 GP120 Antigen Construction of Plasmid Encoding HIV-1 GP120 Antigen

A plasmid vector endoding HIV-1 gp120 was constructed as follows. The vector was constructed starting with a Bluescript (Stratagene, La Jolla, Calif.) plasmid backbone, the human cytomegalovirus (hCMV) immediate early promoter (Fuller et al. (1994) Aids Res. Hum Retroviruses 10:1433) and the SV40 virus late polyadenylation site. The hCMV promoter is contained within a 619 base pair (bp) AccII fragment extending 522 bp upstream and 96 bp downstream from the immediate early transcription initiation site. The SV40 virus late polyadenylation sequence is contained within an approximately 800 bp BamHI-BglII fragment derived from pSV2dhfr (formerly available from Bethesda Research Laboratories, catalogue #5369 SS). Initially, a plasmid encoding HIV-1 gp160, termed “pC-Env” was constructed. This plasmid contains a 2565 bp KpnI-XhoI fragment from LAV-1_{BRU} (ATCC Accession No. 53069, GenBank Accession No. K02013), which begins at the sequence encoding amino acid position #4 of the mature gp160 amino terminus. The env coding sequence fragment was placed immediately downstream of, and fused in frame with a 160 bp synthetic fragment encoding the herpes simplex virus glycoprotein D (gD) signal peptide and none amino acids of the mature gD amino terminus as previously described (Fuller et al. (1994) Aids Res. Hum Retroviruses 10:1433).

The plasmid encoding HIV-1 gp120, termed “pCIA-Env/T” herein, was then constructed as follows. The pCIA-Env/T plasmid encodes a truncated form of HIV-1 gp160, and is identical to the pC-Env construct except that the env coding sequences are truncated at the HindIII site at nucleotide position 8188. This results in a truncated gp160 translation product with the truncation point lying 128 amino acid residues downstream of the gp120/gp41 processing site.

In Vitro Immune Assays

Serum antibody responses to the HIV gp120 antigen were tested using an ELISA assay on specimens collected at week 5 and week 6.5 (ost-prime and post-boost, respectively). For the ELISA, Costar high binding EIA plates were coated with 0.3 μg/well of recombinant HIV gp120 (Intracel) in 50 μl PBS by incubation overnight at 4° C. Plates were washed three times and blocked with 2% BSA in PBS for 2 hours at room temperature. Serial dilutions of serum were added to the coated plates, and incubated at 37° C. for one hour. After washing, the plates were incubated with a 1:1500 dilution of alkaline phosphatase conjugated goat anti-mouse IgG (H+L) (BioRad), followed by color development with p-nitrophenylphosphate (PNPP) (BioRad) and OD reading @ 405 nm.

The amount of antigen-specific IFN-γ secreted by the splenocytes was determined using a cytometric bead assay. 1×10⁶ splenocytes were added to each well of a 96 well plate and were stimulated in medium alone (negative control), or in medium with 1 μg/ml of a HIV gp120peptide having the following sequence: RIQRGPGRAFVITGK (SEQ ID NO: 21). Following a 48 hour incubation at 37° C. in 5% CO₂, supernatants were removed and IFN-gamma levels were measured by a cytometric bead assay (BD Biosciences).

HSV-2 Antigens Construction of Plasmid Encoding HSV-2 ICP27

A DNA vaccine encoding ICP27 was constructed and then combined with various combinations of the present adjuvant plasmid vectors to provide vaccine compositions. After immunization, the immunized animals were challenged with HSV-2 virus, and the protective effect of the various vaccine compositions was determined.

With respect to the construction of the DNA antigen plasmid, standard PCR techniques were used to construct the plasmid. The standard PCR conditions that were used for the construction of the vector were as follows: 1× PCR core buffer with 1.5 mM MgCl₂ (Promega Corp., Madison, Wis.); 0.400 μM of each primer; 200 μM of each dNTP (USB Inc., Cleveland, Ohio); 2.5 μg Taq polymerase (Promega Corp., Madison, Wis.); 1.0 ng template DNA; water to 100 μl; and a mineral oil (Aldrich Chemical Inc., Milwaukee Wis.) overlay. A PTC-200 thermocycler (M. J. Research Inc., Waltham, Mass.) was programmed to run the following routine: 4 minutes @ 95° C.; 30 cycles of (1 minute @ 95C/ 1 minute 15 seconds @ 55C/ 1 minute @ 72° C.); 10 minutes @ 72° C.; 4° C. hold. The amplification products were removed from the PCR reaction using the QIAquick7 PCR Purification Kit (Qiagen Inc., Valencia Calif.) prior to cutting with restriction enzymes (New England Biolabs, Beverly, Mass.).

More specifically, a DNA vaccine plasmid vector encoding the HSV-2 early ICP27 antigen was constructed as follows. HSV is a double-stranded DNA virus having a genome of about 150-160 kbp. The viral genome is packaged within an icosahedral nucleocapsid which is enveloped in a membrane. The membrane (or envelope) includes at least 10 virus-encoded glycoproteins, the most abundant of which are gB, gC, gD, and gE. The viral genome also encodes over 70 other proteins, including a group of approximately five immediate early antigens. These early proteins are synthesized early in the viral replication cycle, in contrast to the envelope glycoproteins which are only made late in the life cycle of the virus. For a review of the molecular structure and organization of HSV, see, for example, Roizman and Sears (1996) “Herpes simplex viruses and their replication” in Fields Virology, 3rd ed., Fields et al. eds., Lippincott-Raven Publishers, Philadelphia, Pa. The HSV-2 ICP27 antigen can be readily obtained from the HSV-2 genome, for example the genomic region spanning from approximately nucleotide 114589 to 134980 of the HSV-2 genome, or an EcoRI fragment that spans nucleotides 110931 to 139697 of the HSV-2 genome. The sequence of the HSV-2 genome is available form published sources, for example the sequence deposited with GenBank under Accession Number NC₁₃001798.

In order to construct the ICP27 vector used in the present study, the ICP27 coding region was PCRed from the HSV-2 genome using the following primers: 5′CGCC ACT CTC TTC CGA CACC3′ (SEQ ID NO:25) and 5′CCAA GAA CAT CAC ACG GAA CC3′ (SEQ ID NO:26) to obtain a nucleotide fragment containing nucleotide sequences 114523-116179 (GenBank) of HSV-2 which correspond to the ICP27 coding region. The ICP27 fragment was then cloned into the multiple cloning region of the pTarget vector (Promega Corp., Madison, Wiss.).

Construction of the PJV7630 Plasmid

The origins of the antigen genes for pPJV7630 are a genomic fragment of HSV-2 strain MS that had been cloned into a cosmid vector called cosmid 23. Cosmid 23 was composed of 3 EcoRI fragments from HSV-2 that spanned from nucleotides 110,931 to 147,530 based on the published sequence (HG52 strain). Cosmid 23 was partially digested with EcoRI and religated and a construct that had only the approximate 28,000 bp fragment (110,931 -139,697) was selected. This molecule was designated OP23. From this molecule 6 modifications were made to alter sequences within the OP23. These were designed to remove non immediate early genes from the HSV-2 sequences and also backbone DNA sequences. One final modification was to replace the backbone sequences with an appropriate antibiotic resistance gene for clinical use. The steps are described below.
1. Bstl 107I and ScaI digest and re-ligation (removes ampicillin resistance gene). Creates OP23-1.
2. NsiI digest and re-ligation to remove SV40 origin of replication. Creates OP23-2.
3. BstXI partial digest and re-ligation to remove regions between ICP27 and ICP0 to make OP23-3.
4. Complete digest with BspHI followed by partial with BsiWI then re-ligation to remove sequences following the ICP22 gene and some backbone sequences. Creates OP23-4.
5. SrfI digest and re-ligation to create OP23-5. Removes sequences between ICP4 and ICP0.
6. BstXI total digest and re-ligation to create OP23-6. Small fragment removed from between ICP27 and ICP0.
7. Replacement of backbone sequences coding for the antibiotic resistance gene, with a fragment containing a Kanamycin resistant gene to create pPJV7630.

The construct pPJV7630 is large (19517 bases) and contains genes encoding the ICP0, ICP4, ICP22 and ICP27 immediate early antigens.

In Vitro Immune Assays Mice

Single cell suspensions were obtained from mouse spleens. Spleens were squeezed through a mesh to produce a single cell suspension and cells were then sedimented, and treated with ACK buffer (Bio Whittaker, Walkersville Md.) to lyse red blood cells. The cells were then washed twice in RPMI 1640 media supplemented with HEPES, 1% glutamine (Bio Whittaker), and 5% heat inactivated fetal calf serum (FCS, Harlan, Indianapolis Ind.). Cells were counted, and resuspended to an appropriate concentration in “Total” media consisting of RPMI 1640 with HEPES and 1% glutamine, supplemented with 5% heat inactivated FCS, 50 μM mercaptoethanol (Gibco-BRL, Long Island N.Y.), gentamycin (Gibco-BRL), 1 mM MEM sodium pyruvate (Gibco-BRL) and MEM non-essential amino acids (Sigma, St. Louis Mo.). For the CD8 specific assays cells were cultured in vitro in the presence of a peptide corresponding to a known CD8 epitope. For ICP27 in BALB/C mice the sequence of the peptide was HGPSLYRTF (QCB Inc). Peptides were made up in DMSO (10 mg/ml) and diluted to 10 μg/ml in culture medium.

For IFN-γ ELISPOTs assays Millipore Multiscreen membrane filtration plates were coated with 50 μL of 15 μg/ml anti-IFN-y antiserum (Pharmingen) in sterile 0.1 M carbonate buffer pH 9.6, overnight at 4° C. Plates were washed 6× with sterile PBS and then blocked with tissue culture medium containing 10% fetal bovine serum (FBS) for 1-2 hr at RT. The medium was removed and spleen cells dispensed into the wells with a total of 1×10⁶ cells per well. For wells in which less than 1×10⁶ cells from immunized animals was added, cells from naive animals were used to bring the total to 1×10⁶. Cells were incubated overnight in a tissue culture incubator in the presence of the peptide as described above. Plates were washed 2× with PBS and 1× with distilled water. This was followed with 3 washes with PBS. Biotinylated anti IFN-γ monoclonal antibody (Pharmingen) was added to the plate (50 ul of a 1 ug/ml solution in PBS) and incubated for 2 hr at RT. Plates were washed 6× with PBS then 50 μL of a Streptavidin Alkaline phosphatase conjugate (1:1000 in PBS, Pharmingen) was added and incubated for 2 hr at RT. Plates were washed 6× with PBS and the color substrate (BioRad) was added and the reaction was allowed to proceed until dark spots appear. The reaction was stopped by washing with water 3×. Plates were air dried and spots counted under a microscope.

In Vivo Assay Mice

An infectious challenge model was used to test the ability of different immunization schedules to protect mice from a lethal challenge with HSV-2. Mice were immunized prior to challenge and for infection were anesthetized and administered with a lethal dose of HSV-2 intranasally in 30 μL of PBS. Mice were followed for 20 days after infection and were scored for sickness and mortality.

In Vitro Immune Assays Domestic Pigs

To isolate peripheral blood mononuclear cells (PBMCs) whole blood was spun through a Histopaque-1077 cushion (3000 rpm for 30 minutes) at room temperature and PBMCs recovered as a band from the gradient. PBMCs were washed 3× in total media and resuspended in 25 mLs of Total media for counting and were resuspended to 1×10⁷ cells/ml in Total media. The ELISPOT assay was carried out as described for mice with the exception that the antigens were pools of peptides derived from overlapping peptide libraries of the HSV-2 antigens, and an anti-IFN antibody pair specific for domestic pig IFN-γ (R&D systems) was used for detection.

InVivo Assay Domestic Pigs

Another assay used to examine immune responses in domestic pigs was a delayed type hypersensitivity (DTH) assay. This assay used both DNA plasmids and protein extracts as antigen sources that were delivered into the pig skin using the XR1 device and needle injections respectively. The area of redness (erythema) surrounding the delivery site of the antigens was measured at 48 hrs after administration as an indicator of a DTH reaction. The antigen delivery into the skin was done 7 days after immunization was complete.

### EXAMPLE 1

Two different plasmids comprising NOIs were used to immunize mice. These were: HSV-2 clinical vaccine multiple gene plasmid (PJV7630) and a single gene plasmid comprising the ICP27 NOI operably linked to the HCMV promoter. The ICP27 NOI encodes the dominant epitope found in PJV7630 and the graphs represent CD8 responses specific for this protein.

NOI Administration Schedule

The NOI was administered 1, 2 or 4 times during 1 week either at day 7 (D 7), days 0 and 7 (D 0,7) or days 0, 2, 4, and 7 (D 0,2,4,7) respectively. Either 1 or 2 shots per NOI administration were given, and in one group LT adjuvant were co-administered with the NOI.

Results

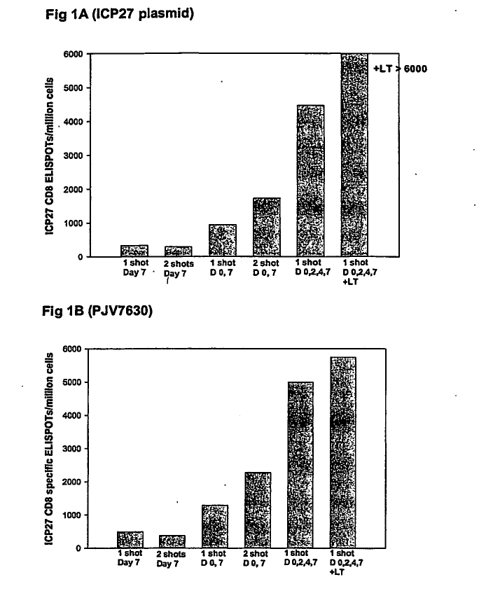

The ICP27 gene is the dominant antigen found in PJV7630 and the FIGS. 1A and 1B represent CD8 responses specific for this target antigen.

Typically both plasmids PJV7630 (FIG. 1) and ICP27 (FIG. 1A) generate 500 ELISPOTs/million cells after only one NOI administration and 1500 ELISPOTs/million cells after two NOI administrations. When two NOI administrations are followed with the LT genetic adjuvant approximately 3500 ELISPOTs are found. Above 3500 ELISPOTs/million cells were obtained after four NOI administrations even in the absence of LT adjuvant but the LT genetic adjuvant will enhance the responses as well. In FIG. 1A the response from the LT co-administered with the NOI was not measured as the results were off-scale.

Summary

Clustering NOI administrations had been found to generate enhanced cellular immune responses based on ICP27 specific CD8 IFN-gamma ELISPOTs measured in mice immunized with PJV7630.

### EXAMPLE 2

The purpose of following three experiments was to assess whether an enhanced CMI response resulting from clustered NOI administrations of a single gene plasmid correlated with enhanced protection from lethal challenge.

Methodology

Plasmid PJV7630 was administered to mice over a one week period. Either one (day 7), two (days 0 and 7) or 4 (days 0, 2, 4 and 7) NOI administrations were given over the period of one week. One group received 2 doses at each immunization but the majority of mice received a single dose of PJV7630 at each immunization.

Results

On the graphs the labelling is “number of administrations × number of doses per administration” so that 4×1 are animals given 4 NOI administrations with a single dose at each administration. Following a week of NOI administration, the animals were rested either 1 week or 2 weeks before infected with virus. The dose of virus was approximately 5 times the LD50.

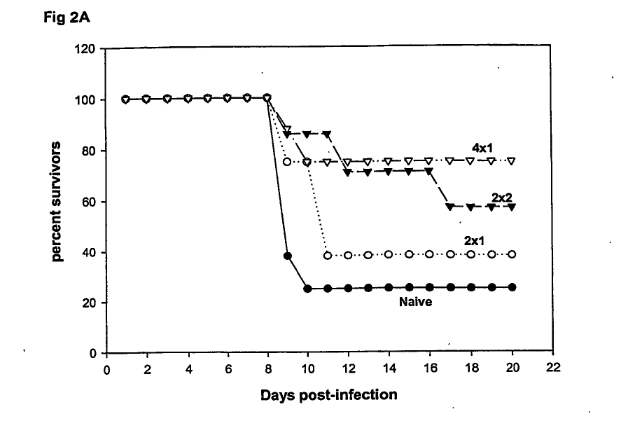

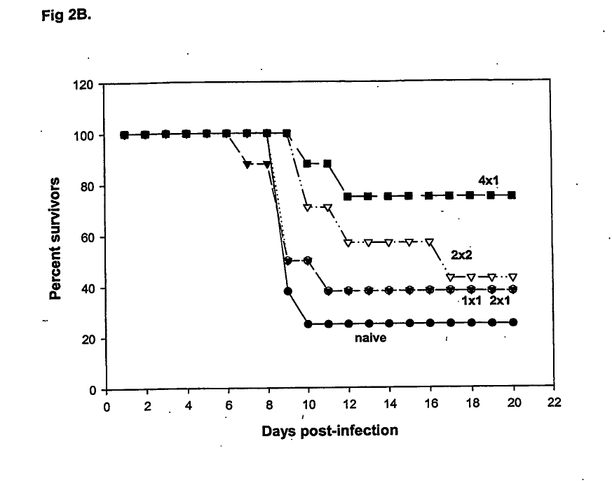

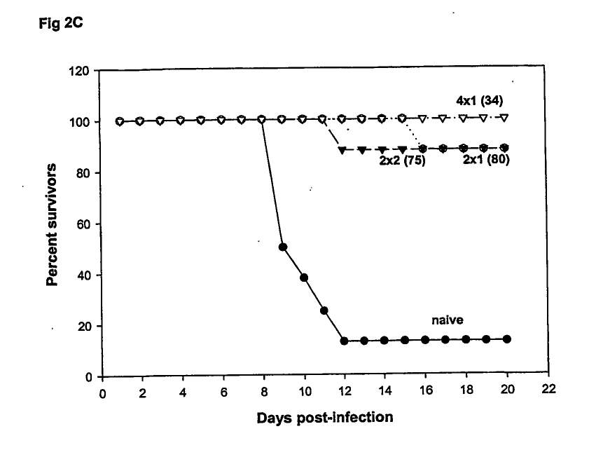

FIG. 2A shows results from C57B1/6 mice after 2 weeks rest. The results clearly demonstrate that a 4×1 schedule (ie 4 administrations ×1 dose per administration) was more protective. This result does not appear to result from the increased dose since 2×2 (ie 2 NOI administrations ×2 doses) also has 4 doses in total.

FIG. 2B shows results from C57B1/6 mice after I week rest. It is clear from FIG. 2B that virtually the same results were obtained as for the two week rest shown in FIG. 2A. However, in FIG. 2B there is an additional group with just one NOI administration.

FIG. 2C shows results from Balb/c mice after 1 week rest. It is clear from the results that the challenge dose was not high enough to get a clear difference in mortality, but there were differences in sickness. The numbers in brackets are sickness scores with the higher the number the sicker the animals. The results are in line with those from FIGS. 2A and 2B which show that 4×1 (ie 4 administrations ×one dose) confer the best protection.

Summary

The clustering of 4 administrations ×one dose per administration in 1 week rapidly generates a strong protective CMI response that is stronger than single administrations or using more doses per administration.

### EXAMPLE 3

The purpose of this experiment was to vary the time interval between DNA administrations of a single gene plasmid.

Methodology

All the mice were given a total of 4 administrations with differences between the date of receipt of each administration and time interval between administrations. Mice had 6, 4, 2, 1 or 0 day intervals between administrations (ie 0 had 4 shots on one day). The final administration of each schedule was given on the same calendar day and then all animals were sacrificed 7 days after the final administration.

Results

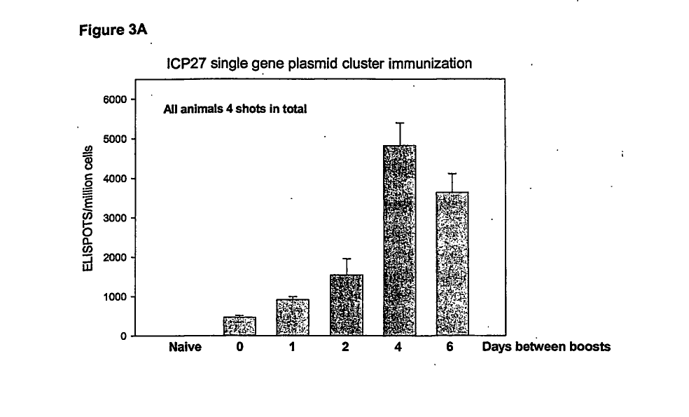

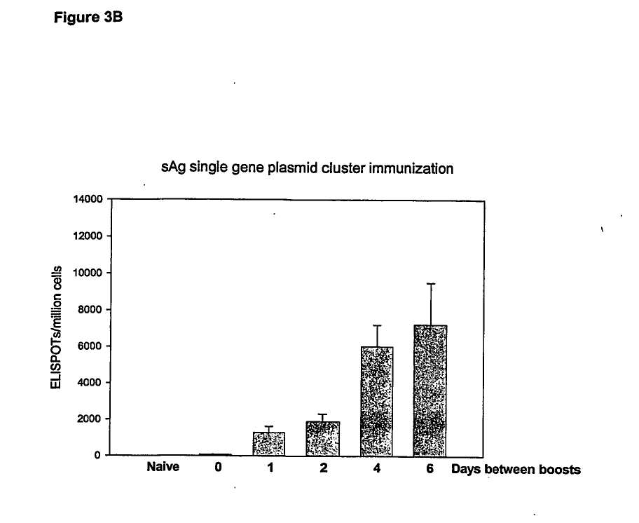

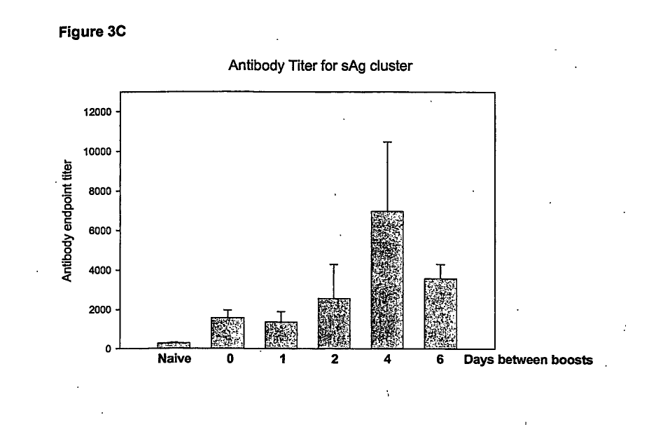

FIG. 3A provides CD8 ELISPOTs results for 0, 1, 2, 4 and 6 day time intervals between clustered administrations of a ICP27 single gene plasmid. The results show that increasing the time interval between administrations enhances the CD8 ELISPOT results with a maximum results obtained when there was a 4 day interval (ie 96hours) between NOI administrations.

FIG. 3B provides CD8 ELISPOTs results for 0, 1, 2, 4 and 6 day time intervals between clustered administrations of a HbsAg single gene plasmid. The results show that increasing the time interval between administrations enhances the CD8 ELISPOT results with maximum results obtained when there was a 4 to 6 day interval between the NOI administrations.

Summary

Examination of time intervals between clustered NOI administrations in mice demonstrated that when 4 NOI administrations are given, optimal responses in terms of a CD8+T cell response are obtained when the NOI administrations are spaced 4 or 6 days apart.

FIG. 3C provides antibody results from clustered administration of the HbsAg single gene plasmid. The antibody titres obtained are quite weak. These results suggest that clustered NOI administrations with 0, 1, 2, 4, or 6 day intervals between administrations does not seem to significantly enhance antibody titer even though the CD8+T cell response is enhanced.

### EXAMPLE 4

The purpose of this experiment was to assess the CMI response in terms of CD8+T cell response for clustered NOI administration of a multi gene plasmid (PJV7630).

Methodology

Animals were given 4 NOI administrations in total but the time interval between NOI administrations varied. The final administration for each group was on the same day (the start of the NOI administrations varied) and responses were measured 1 week and 3 weeks after completion of the administrations. The 3-week sampling was added to minimize the effect that the different schedules had on the timing of administrations. For example, animals getting 4 NOI administrations with a 6-day interval between administrations had an 18 days time interval between the first and fourth administration whereas animals with the 0 time interval between administrations would have had all shots on day 18.

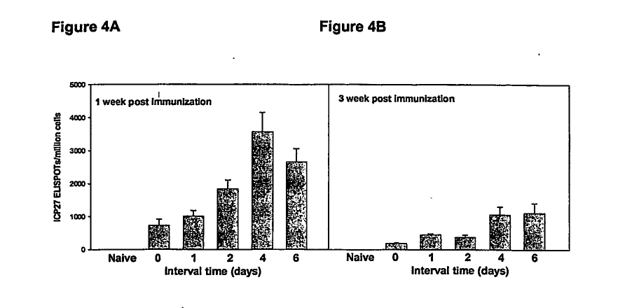

Results The results in FIGS. 4A and 4B show the cellular responses as measured by an ICP27 specific CD8 IFN-gamma ELISPOT. The time between NOI administrations is labelled on the graph. All animals received 4 shots in total. It is clear that the results from the multi gene plasmid (PJV7630) paralleled the results from the initial experiments using the single gene plasmid (ICP27). In this regard, the 4 and 6-day time intervals between administrations gave optimal response in terms of ELISPOT measurements (see FIG. 3C) and this held at 3 weeks but responses had dropped about 3 fold by that time (see FIG. 3D).

### EXAMPLE 5

The purpose of this experiment was to determine to determine if there is any synergy between the use of genetic adjuvants and clustered NOI administration schedule in enhancing humoral and cell mediated immune (CMI) responses to a relatively weak antigen such as HIV-1gp120.

Method

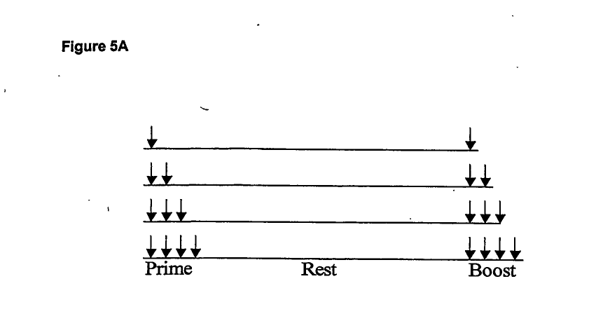

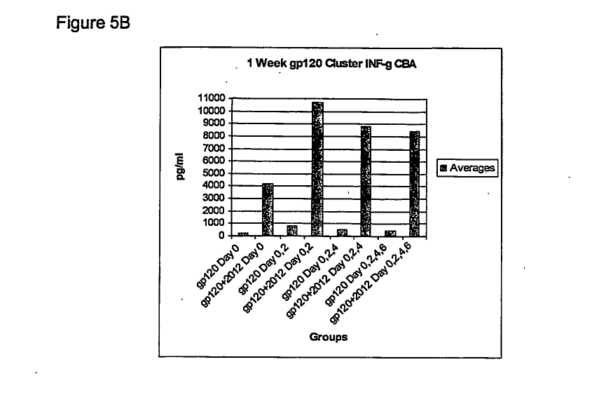

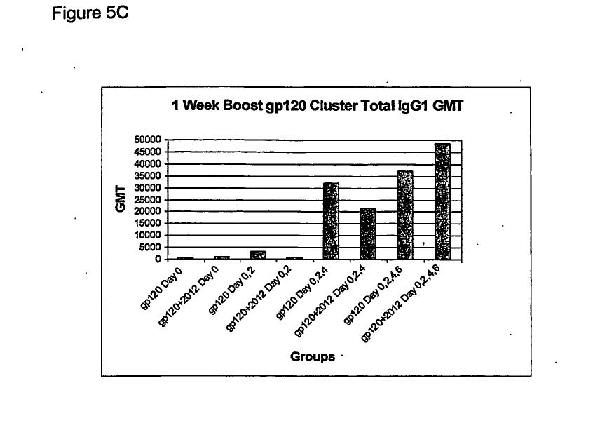

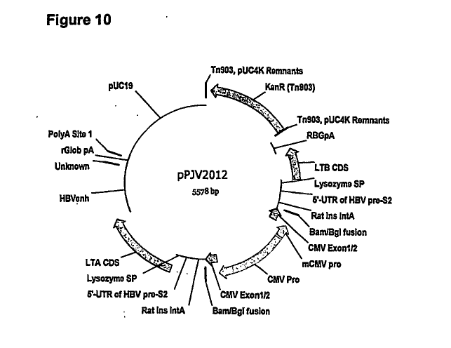

This experiment involves the administration of at least two administrations of the gp120 antigen in mice in which each “administration” is made up of a cluster of 1 to 4 XR1 administrations. See schematic diagram below in FIG. 5A. In addition, the resting period between administrations is one week. An LT A+B genetic adjuvant (pPJV2012) was used. A map of the pPJV2012 plasmid is provided in FIG. 10. The pPJV2012 plasmid was prepared by cloning the LT genes encoding the LTA and LTB subunit proteins into plasmids pPJV-2004 and pPJV-2005 respectively as described in WO 03/004055. The genes encoding the LTA and the LTB subunit proteins were then cut from the original plasmids and inserted into a single plasmid to make the pPJV2012 plasmid.

Results

FIG. 5B shows data obtained from animal groups with a 7 day time interval between NOI administrations. The number of XR1 administrations in each cluster was varied as was the presence or absence of the LT A+B genetic adjuvant (pPJV2012). The results obtained indicate that the presence or absence of the genetic adjuvant had the strongest influence on cellular responses (IFN-gamma production). Figure SB clearly demonstrates that the LT-enhanced responses were strongest when the NOI administration schedule was clustered into two administrations. These results demonstrate that the clustered administration schedule makes a significant contribution to the overall cellular response obtained with the genetic LT adjuvant.

FIG. 5C demonstrates that, in contrast to the CMI response, clustered administrations were shown to have the greatest impact on the strength of total antibody responses. As illustrated in FIG. 5C, very strong gp120-specific titers (5-10-fold higher than previously encountered) were elicited using 4 administrations per cluster with and without the genetic adjuvant vector. Importantly, the presence of the genetic adjuvant influenced the balance in the IgG1-to-IgG2 a subclasses, but was not required to elicit strong antibody titers (not shown).

Summary

The results demonstrate that when an NOI encoding a weak antigen is co-administered with an NOI encoding an adjuvant, then the maximum CMI response in terms of Interferon gamma release is obtained after two NOI administrations. In contrast, a strong humoral immune response is obtained either with or without adjuvant with a cluster of four NOI administrations with a time interval of about 48 hours between administrations.

### EXAMPLE 6

The purpose of this experiment was to determine if immunization of domestic pigs with pPJV7630 would generate cellular immune responses as judged by IFN-γ ELISPOTs and DTH responses.

Method

For this experiment domestic pigs were administered with either pPJV7630 or a placebo (gold alone) by the XR1 device. Pigs were administered with two doses for each immunization and had a cluster schedule of 4 immunizations over a one week period. Thus each pig was given a total of 8 doses of vaccine in a cluster. A second cluster immunization was initiated 28 days after the end of the first cluster.

Results

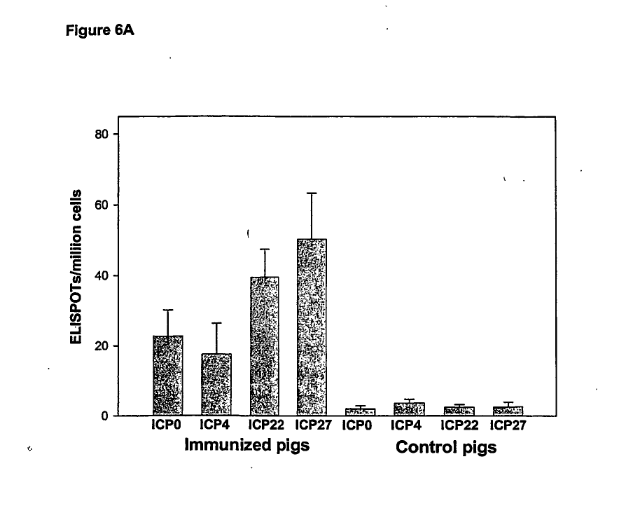

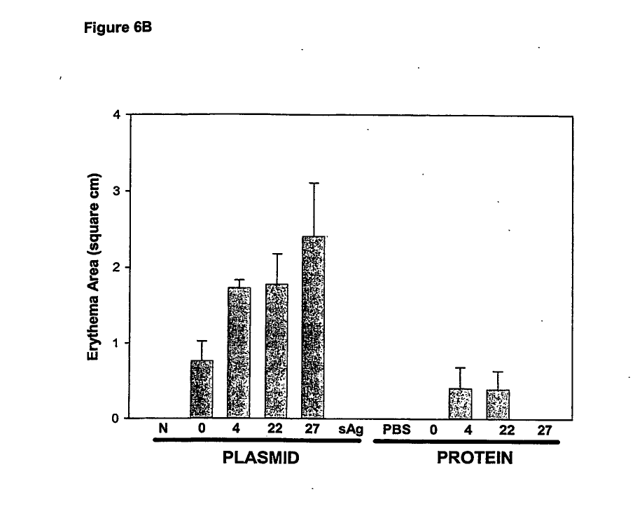

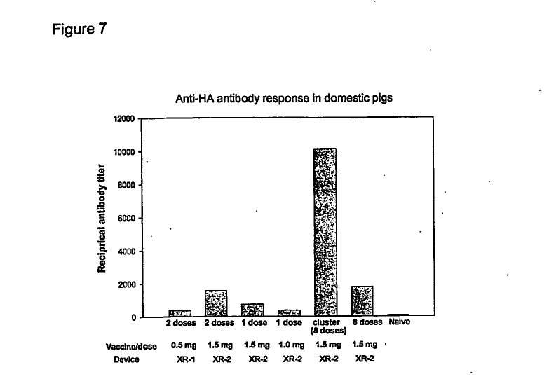

FIG. 6A shows IFN-γ ELISPOT data obtained from animals following the first cluster immunization. Values are the means from 8 immunized animals and 8 control animals. The data shows that the cluster immunization schedule was able to raise cellular immune responses in domestic pigs which are considered to be a difficult model for measuring immunogenicity. The control pigs all showed background levels of ELISPOTs.

FIG. 6B shows the average area of erythema present at the site of antigen administration in pigs (4 animals) immunized with pPJV7630 (control animals are not included on the graph). The antigens were given 7 days after the immunizations were complete and the pigs had received two cluster immunizations. The presence of the erythema 48 hours after administration of antigen indicates that this is a DTH reaction. The results show that the DTH response is antigen specific as the null plasmid (N) or an irrelevant antigen (sAg) the Hepatitis B surface antigen plasmid do not induce the DTHI reaction, whereas plasmids expressing the vaccine antigens (0, 4, 22, 27) show good DTH responses. In pigs given placebo only (data not shown), no DTH reactions were found for any of the antigens verifying that the responses in 6B are the result of immunization with pPJV7630. At sites where protein extracts were injected there were several good DTH measures for the ICP22 and ICP4 proteins, but none for the control PBS solution and the ICPO and ICP27 proteins. Because only 5 μg of protein extract was available for injection, and up to 100 μg may be used to elicit a DTH response, the low response may be related to the administered dose.

Summary

In the domestic pig model a cluster immunization was found to be able to induce cellular immune responses against the vaccine antigens. The domestic pig is not considered to be a good model for raising immune responses but the cluster immunization had the ability to raise cellular immune responses.

### EXAMPLE 7

A domestic pig study was carried out to examine effect of dosing and device on the sponse to the ha protein expressed from pPJV1671 as detailed in Table 1 below. (The XR particle acceleration device is described above).

TABLE 1

| Cohort | | |
|---|---|---|
| (Animal umbers) | Vaccine | Number of Shots |
| 1 | pPJV1671 Gun # 49 | 2 |
| (1-8) | 0.5 mg Au/shot | |
| 2 | pPJV1671 XR-2 11/16 | 2 |
| (9-16) | 1.5 mg Au/shot | |
| 3 | pPJV1671 XR-2 11/16 | 1 |
| (17-24) | 1.5 mg Au/shot | |
| 4 | pPJV1671 XR-2 11/16 | 1 |
| (25-32) | 1.0 mg Au/shot | |
| 5 | pPJV1671 XR-2 11/16 | 2 × 4 |
| (38-40) | Cluster immunization* | |
| | 1.5 mg Au/shot | |
| 6 | pPJV1671 XR-2 11/16 | 8 |
| (41-48) | 1.5 mg Au/shot | |
| 7 | Negative Control | |
| (49-56) | | |

*2 shots on alternating days (Days 1, 3, 5 and 8)

Animals were primed and boosted with vaccines at 4 weeks. Blood was taken at various time points and graphed below are antibody titers 2 weeks after the boost. Two groups were given a total of 8 doses at each immunization, either by cluster or all at one time. The animals immunized with cluster had a higher level of serum antibody.

Antibody titers

Antibody titers were measured by an ELISA following standard procedures using 200 hemagglutination units/well of Sarkosyl-disrupted purified Sw/IN virus diluted in phosphate-buffered saline. The swine antibodies were measure directly by using a goat anti-swine immunoglobulin G alkaline phosphatase conjugate.

Plasmid pPJV1671

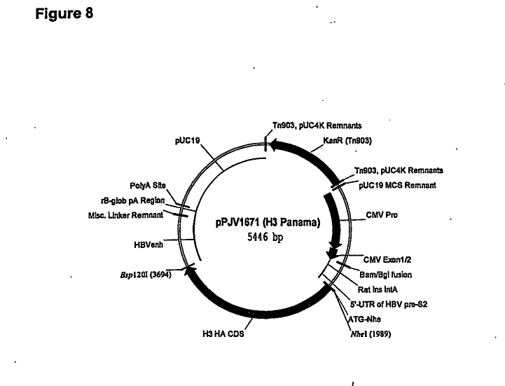

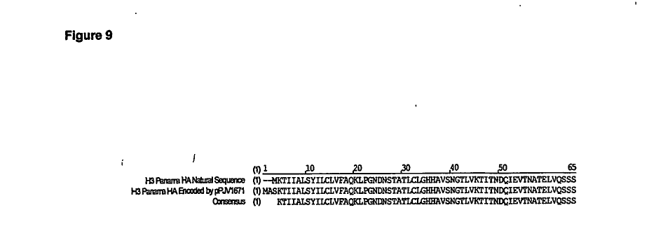

Plasmid pPJV1671, as shown in FIG. 8, is a human DNA vaccine vector encoding the hemagglutinin (HA) antigen of influenza A/Panama/2007/99 (H3N2). The HA coding sequence was obtained by a standard reverse transcriptase / polymerase chain reaction (RT-PCR) cloning technique using a sample of A/Panama/2007/99 virus obtained from the CDC as a source of template RNA. The following steps were employed in developing the final pPJV1671 HA DNA vaccine vector:
RT-PCR production of dsDNA fragment of RNA segment #4 of A/Panama/2007/99 (H3N2).
Propagation of RNA segment #4 DNA clone in a standard pUCl 9-based vector in E. coli.
Sequence analysis of the H3 Panama HA coding sequence within the RNA segment 4 clone.
A second PCR reaction to generate a DNA fragment containing the H3 Panama coding sequence (without its ATG codon) with ends compatible with the pPJV7563 DNA vaccine vector (Nhe I and Bsp 120I).

Insertion of the H3 Panama HA coding sequence into pPJV7563 yielding the final pPJV1671 H3 Panama HA DNA vaccine vector that conform to the Kozak consensus. Use of the vector-supplied ATG codon (via insertion at the Nhe I site) results in a minor 2-amino acid insertion at the amino terminus of the coding sequence of the HA gene as depicted in FIG. 9.

Summary

This study demonstrates that cluster immunisation significantly improved the antibody response in a domestic pig model.

Plasmid pPJV7563

Construction of pPJV7563

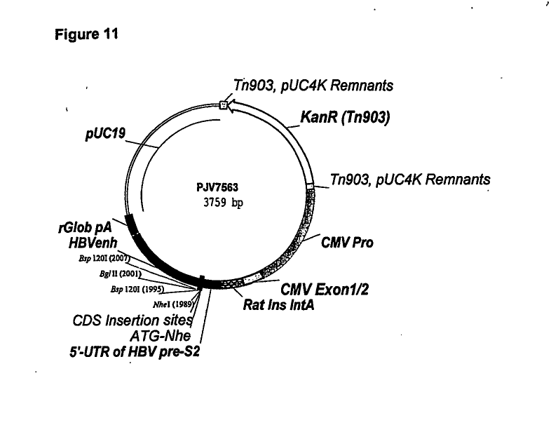

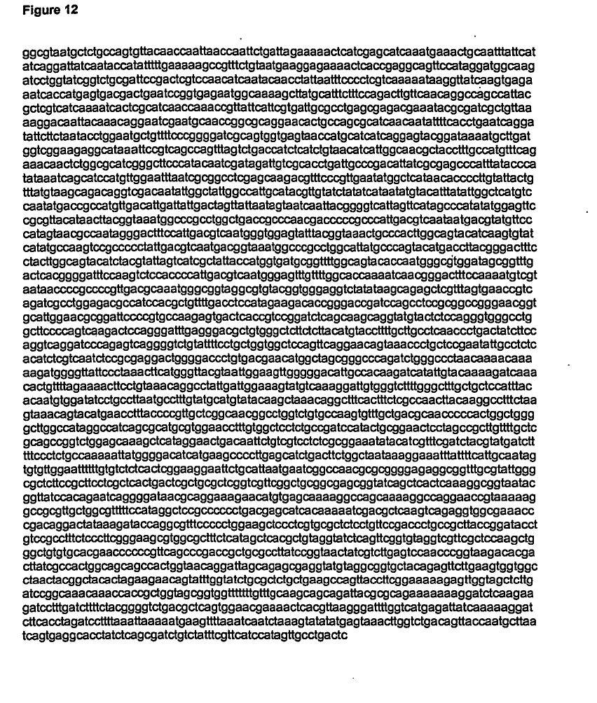

A pPJV7563 plasmid map is provided in FIG. 11. The base composition for the pPJV7563 plasmid is provided in FIG. 12. The components and their position in the plasmid pPJV7563 are as follows:
1-44 Transposon 903 sequences
45-860 Kanamycin resistance coding sequence from Transposon 903
861-896 Transposon 903 sequences
897-902 Sall site
903-1587 CMV promoter
1588-1718 untranslated leader sequence from the immediate-early gene of CMV
1719-1724 Fusion of BamHl and BglII restriction enzymes
1725-1857 Rat insulin intron A
1858-1863 BamH1 site
1864-1984 HBV surface antigen 5′-untranslated leader
1985-1993 Synthetic start codon/ Nhe1 cloning site
1994-2011 Synthetic cloning sites
2012-2544 HBV enhancer
2545-2555 Old vector sequence. No hits against NCBI databases
2556-2686 Rabbit beta-globin polyadenylation region
2687-3759 pUC19 vector sequence

The pPJV7563 plasmid was prepared as follows:

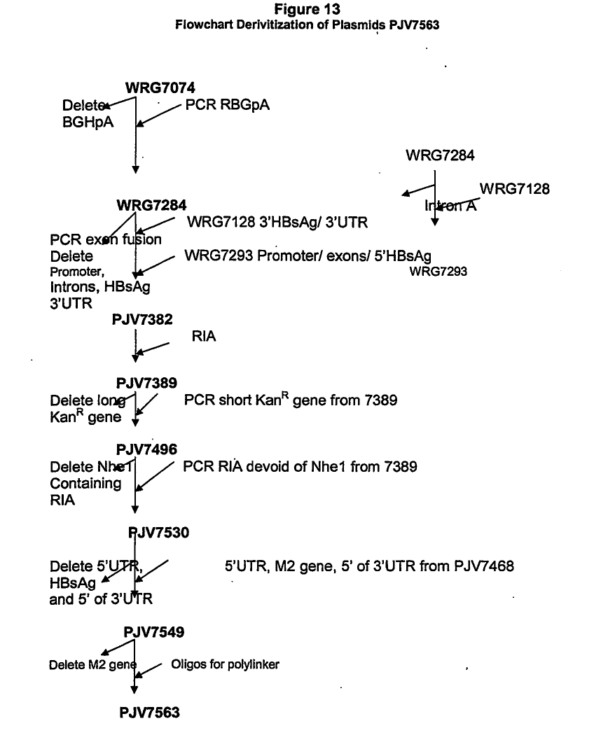

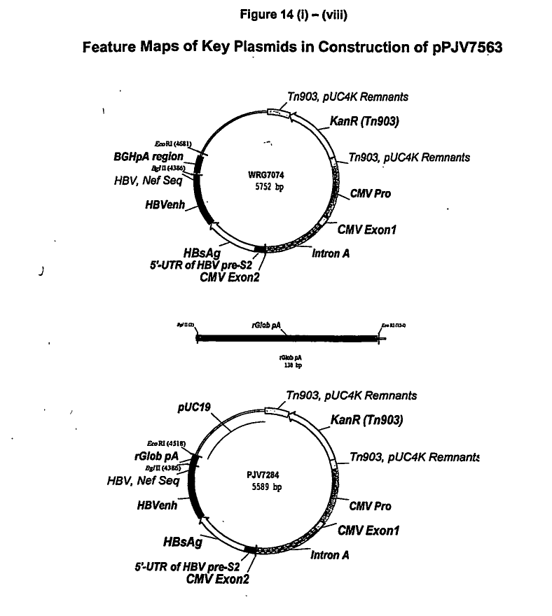

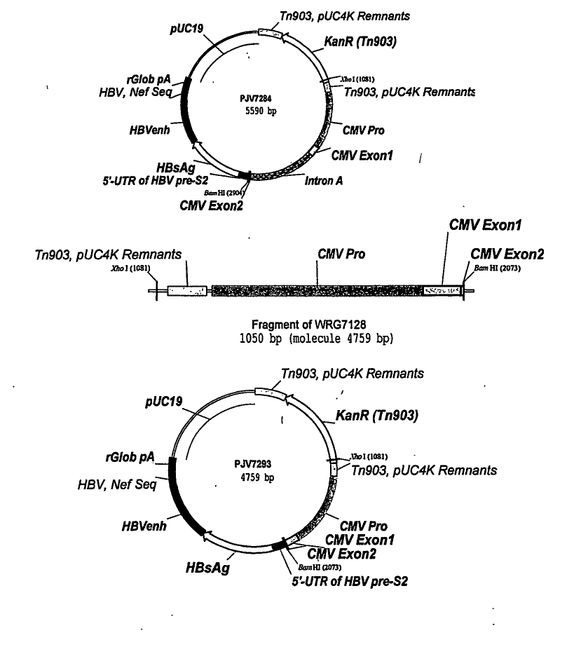

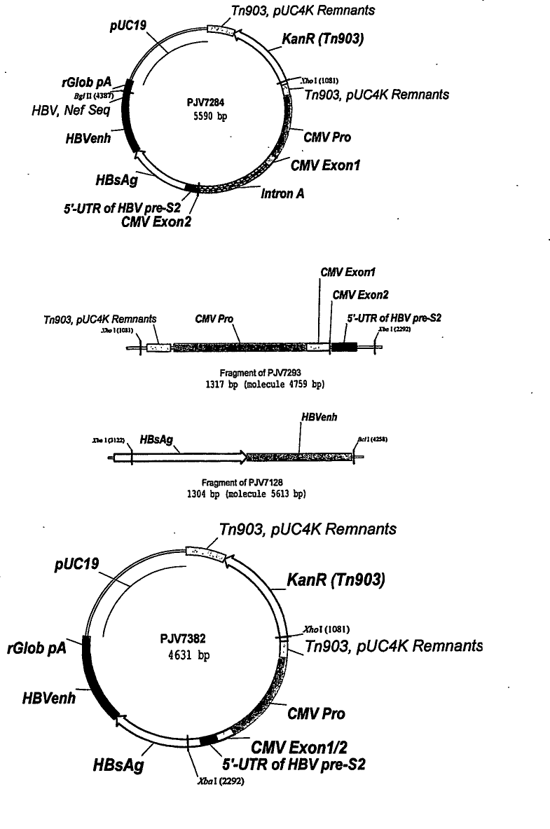

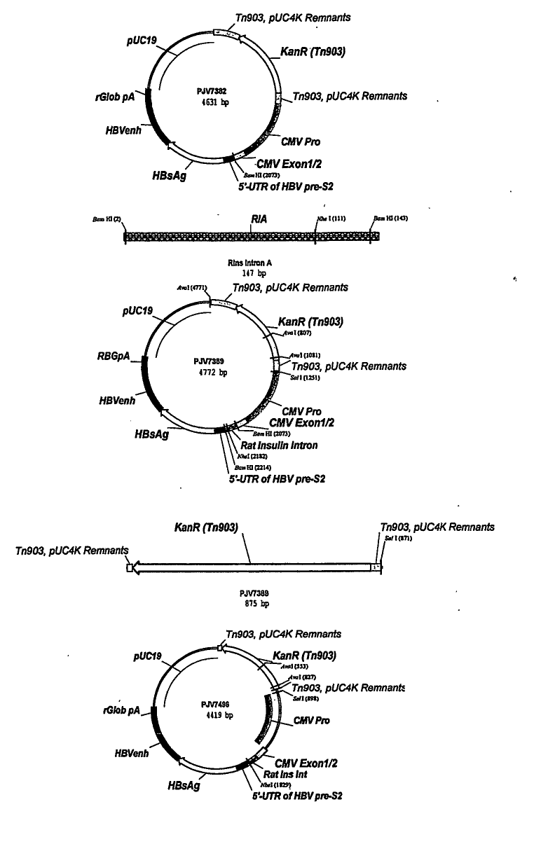

Description of FIG. 13. Flowchart Outlining the Construction of PJV7563:

The bovine growth hormone polyadenylation signal (BGHpA) in pWRG7074 was replaced by the rabbit beta-globulin polyadenylation signal (RBGpA), resulting in pWRG7284. The intron A of CMV was removed from pWRG7284 by replacing all the CMV sequences with a pWRG7128 derived PCR fragment containing the CMV promoter and exon1/ 2 fusion. This resulted in pWRG7293.

The CMV and HBV sequences were removed from pWRG7284 and replaced with the CMV and 5′-HBV sequences from pWRG7293 and the 3′-HBV sequences from pWRG7128. The SIV nef gene sequence was removed at this step, resulting in pPJV7382 pPJV7382 was fer engineered by adding the rat insulin intron A (RIA) to create pPJV7389. The Kanamycin resistance (Kan^{R}) gene in pPJV7389 was replaced with a shortened version to remove unneeded sequences from both ends of this gene, resulting in pPJV7496. The Nhe Isite in the RIA was cured from pPJV7496, yielding pPJV7530.

The HBV sequences through the 5′ region of the 3′-UTR in pPJV7530 were removed and replaced by the HBV 5′-UTR, flu M2 gene and the 5′-region of the 3′-UTR from pPJV7468, yielding PJV7549. PJV has determined that retention of the HBVenh and HBV 5′ UTR regions from WRG7128 in vectors encoding a variety of antigens can reproducibly improve both antigen expression and immunogenicity. These are now common elements in PJV's DNA vaccine vectors. The M2 gene was then deleted from pPJV7549 and replaced by oligonucleotides that formed a polylinker. This manipulation yielded pPJV7563, an expression vector that is able to accept other coding sequences.

Construction of Plasmid pWRG7074. Parent Vector of PJV7563

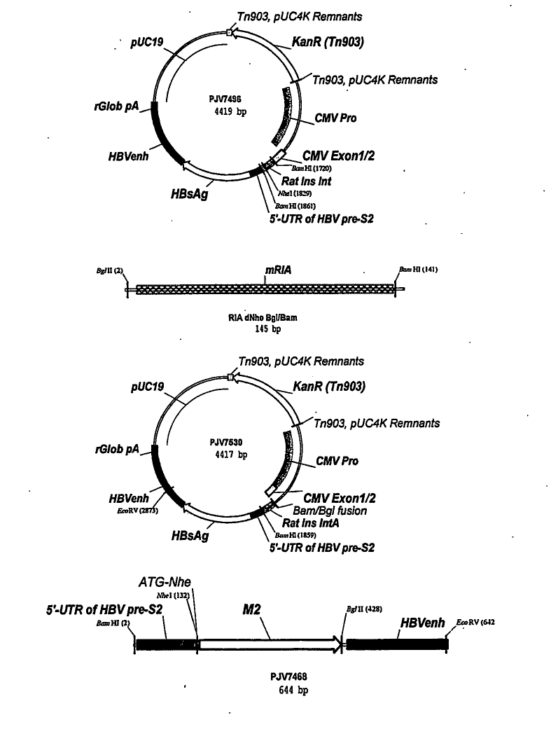

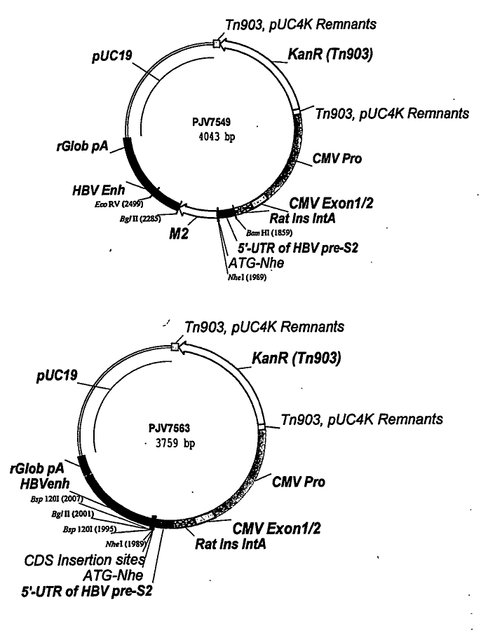

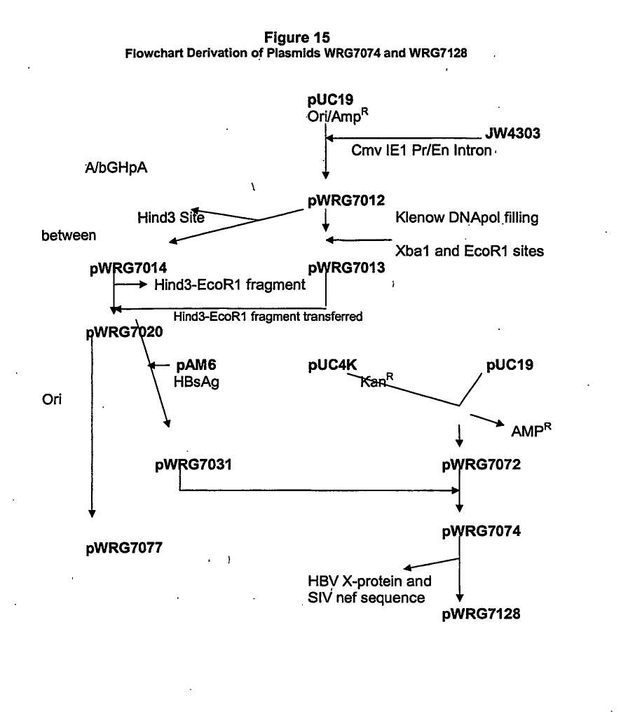

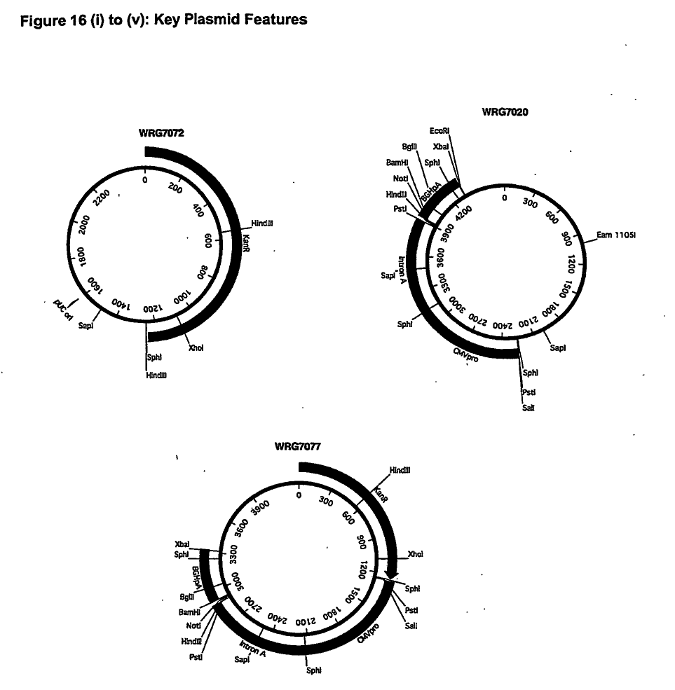

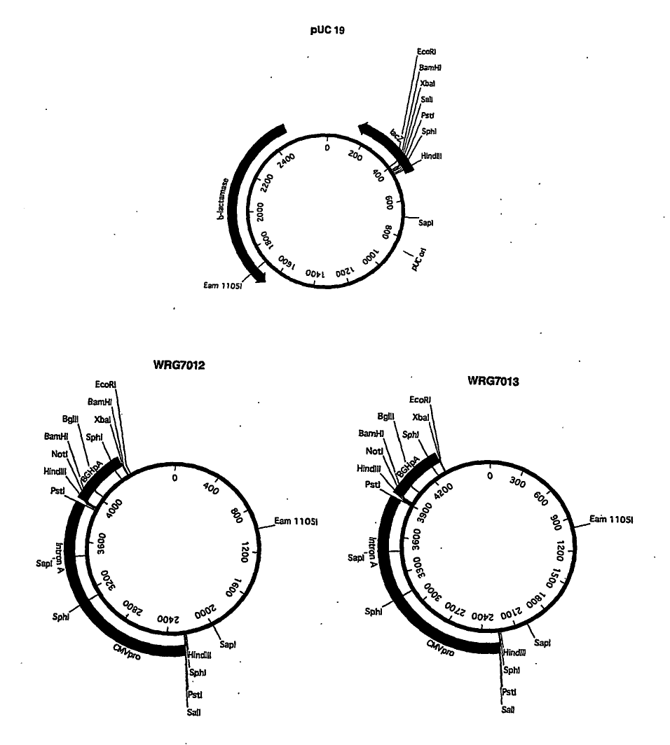

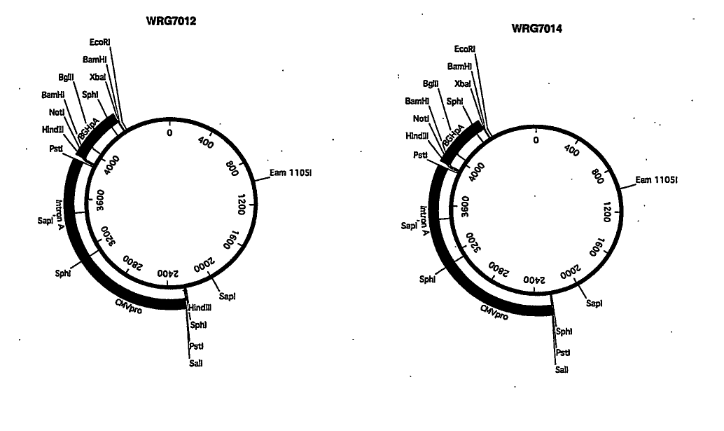

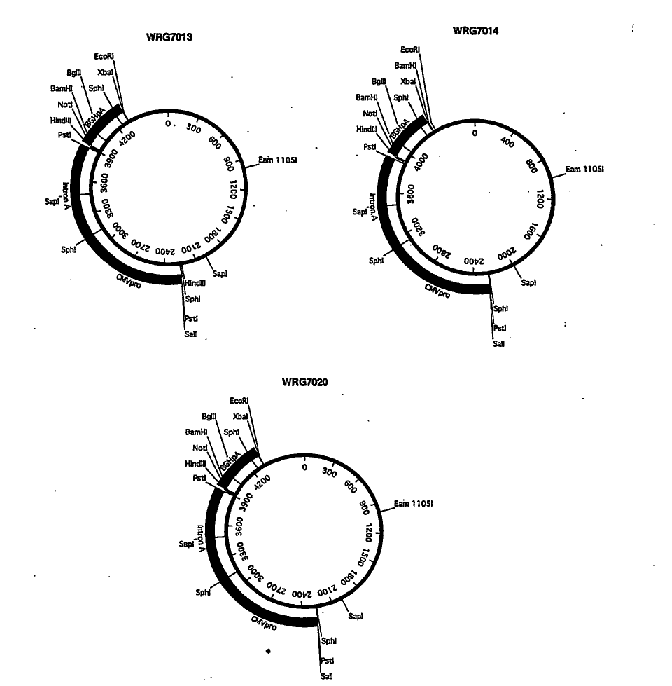

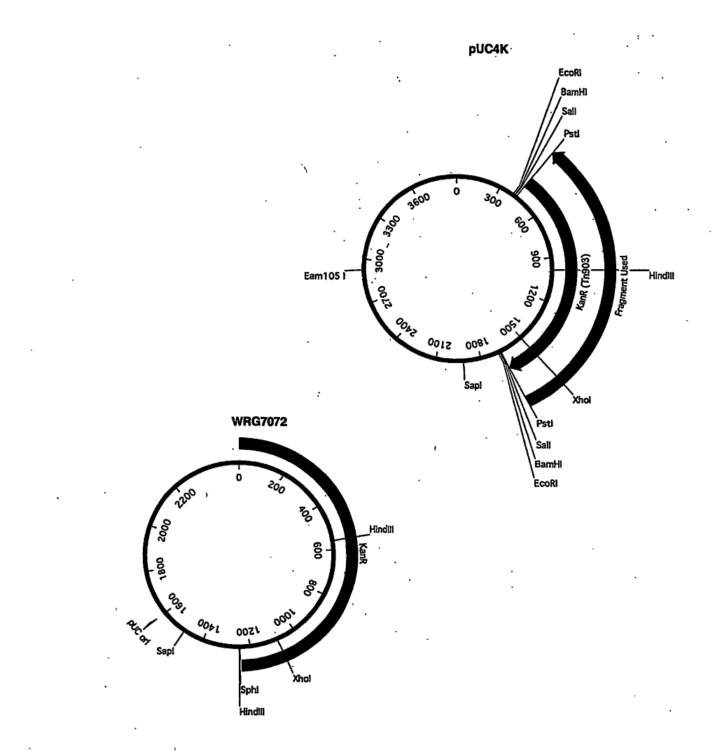

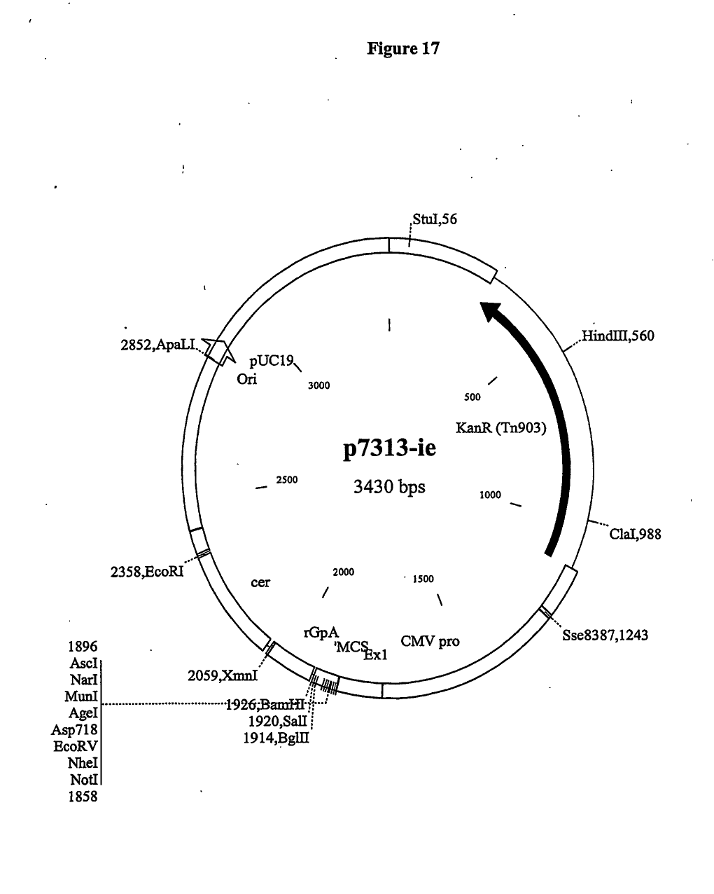

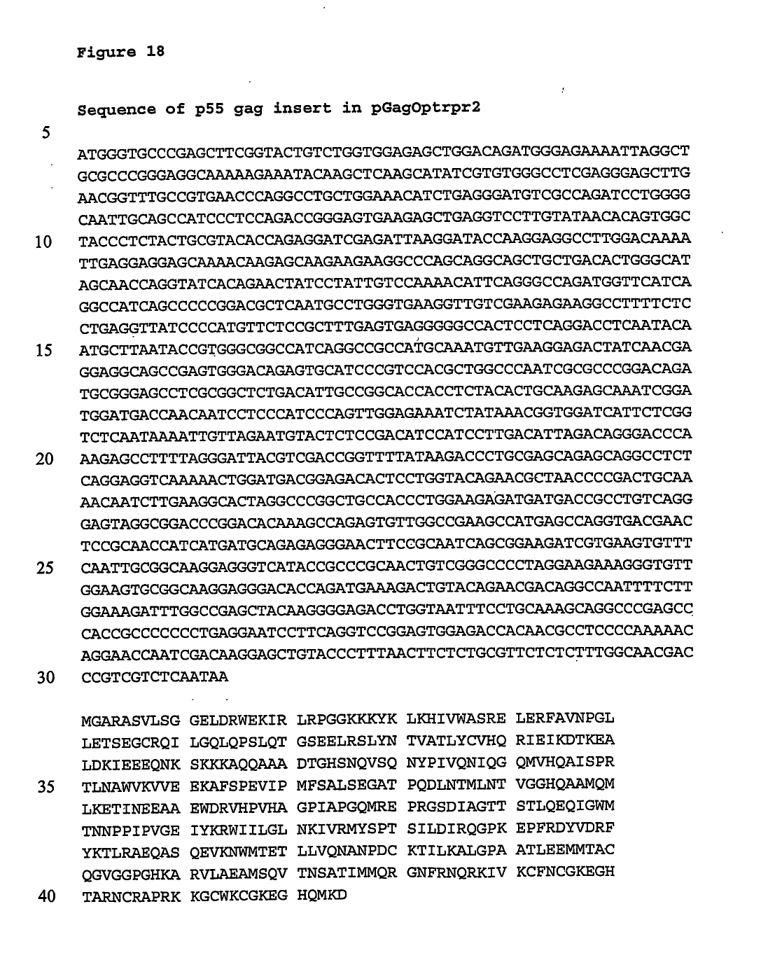

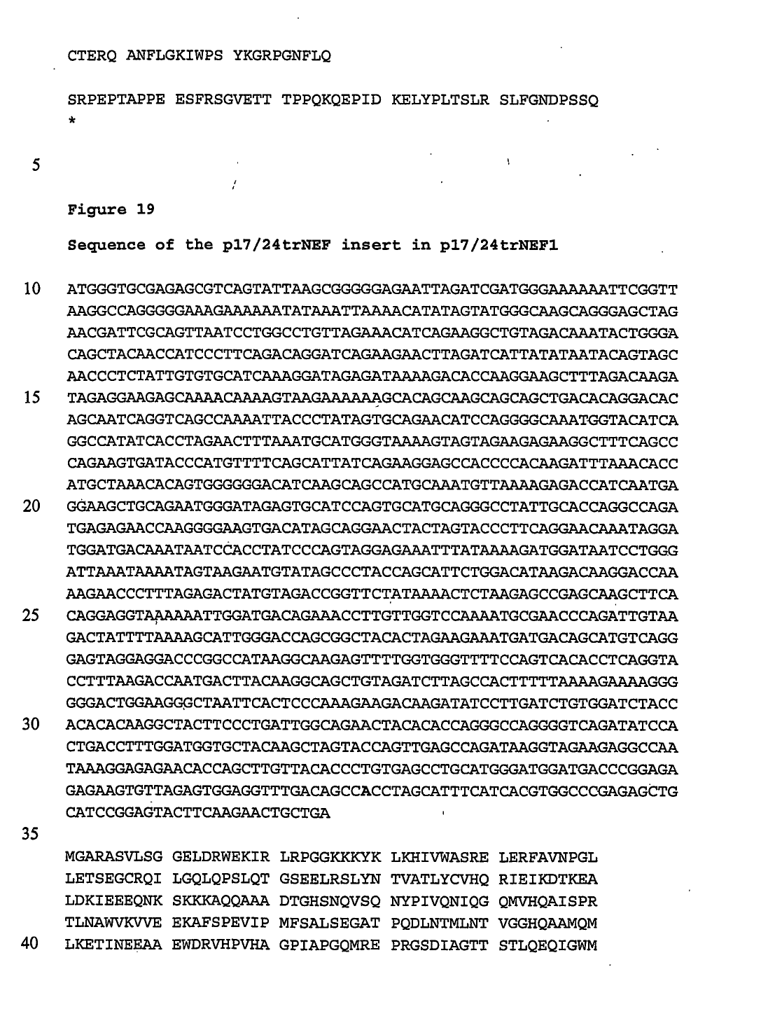

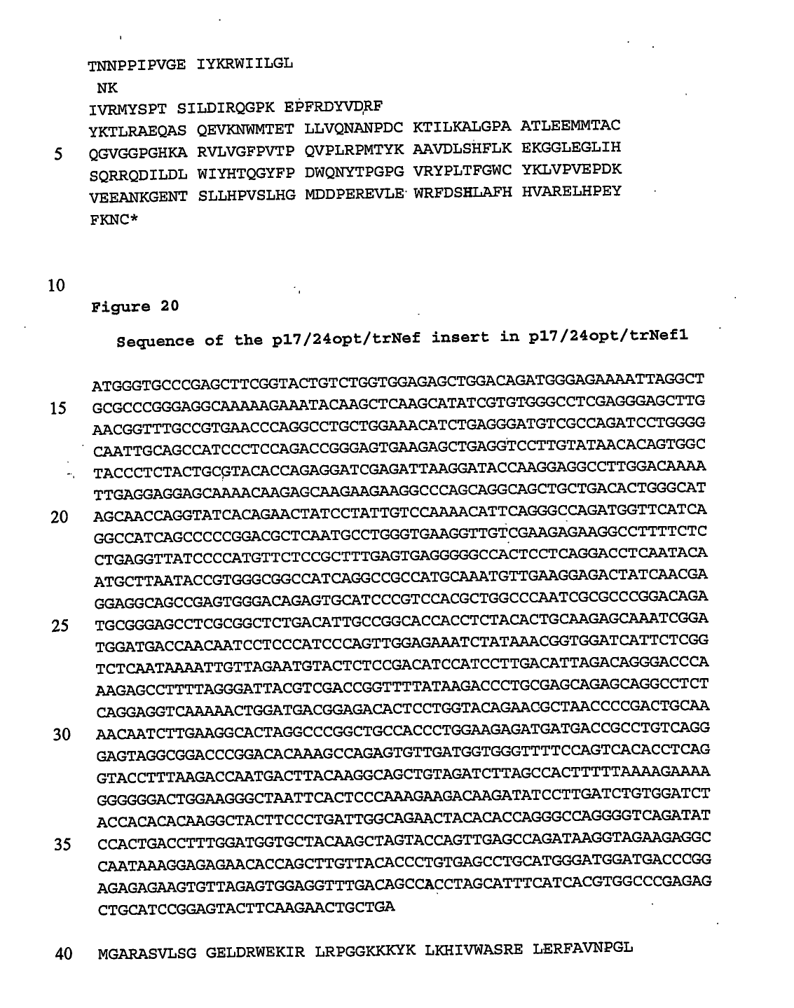

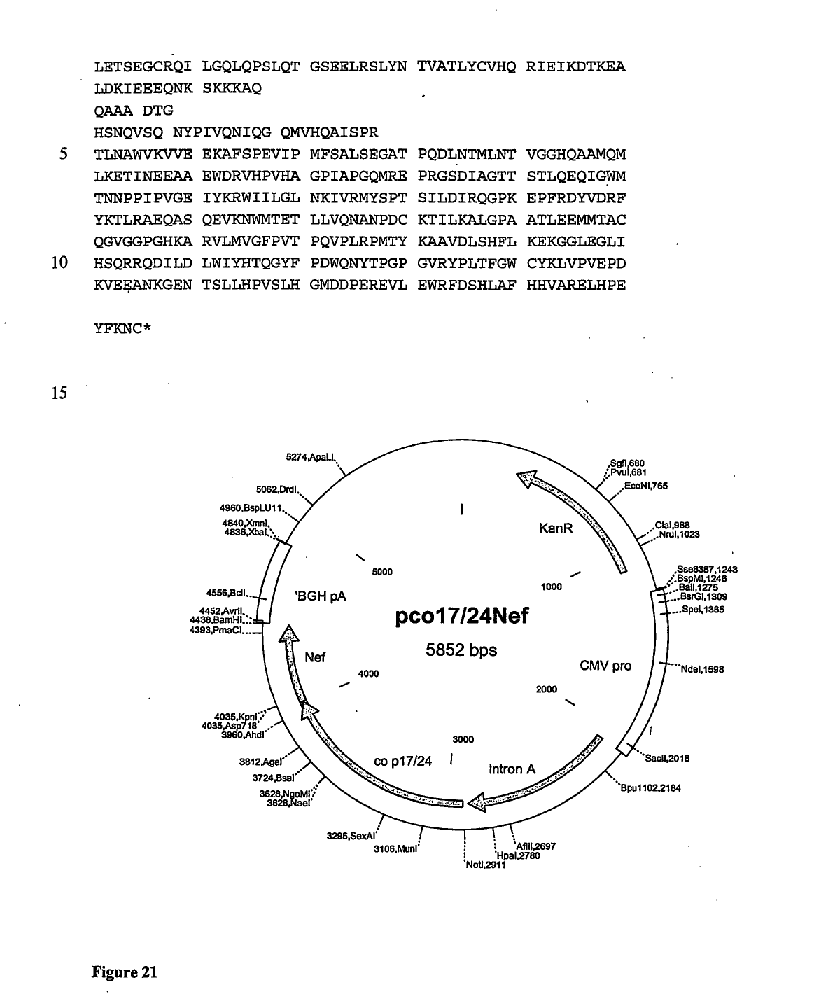

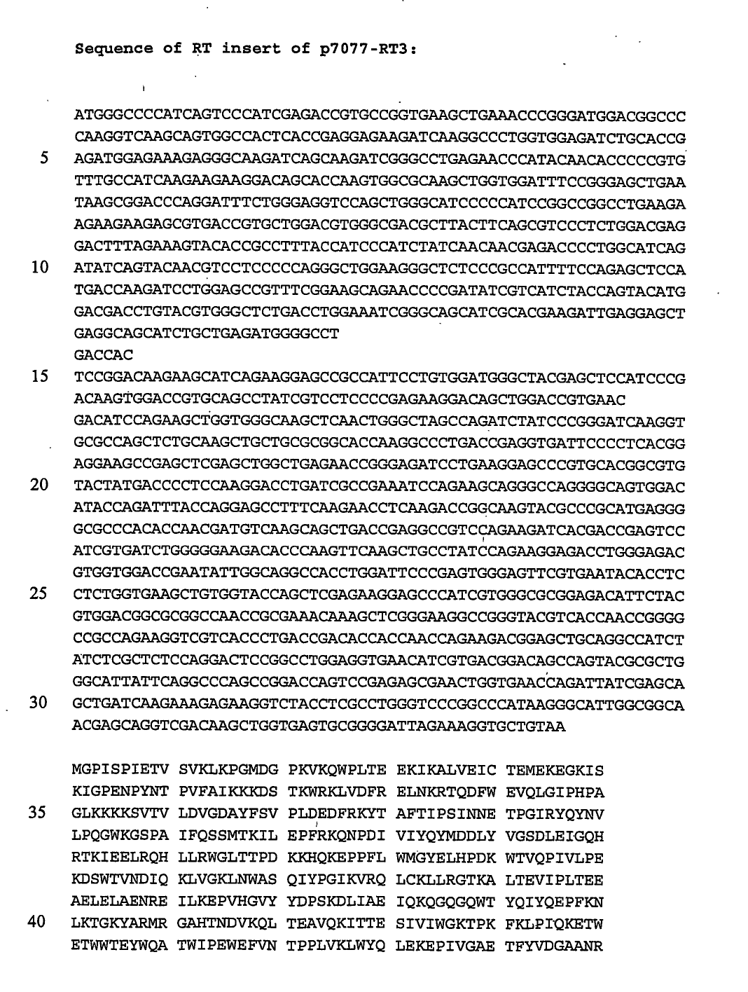

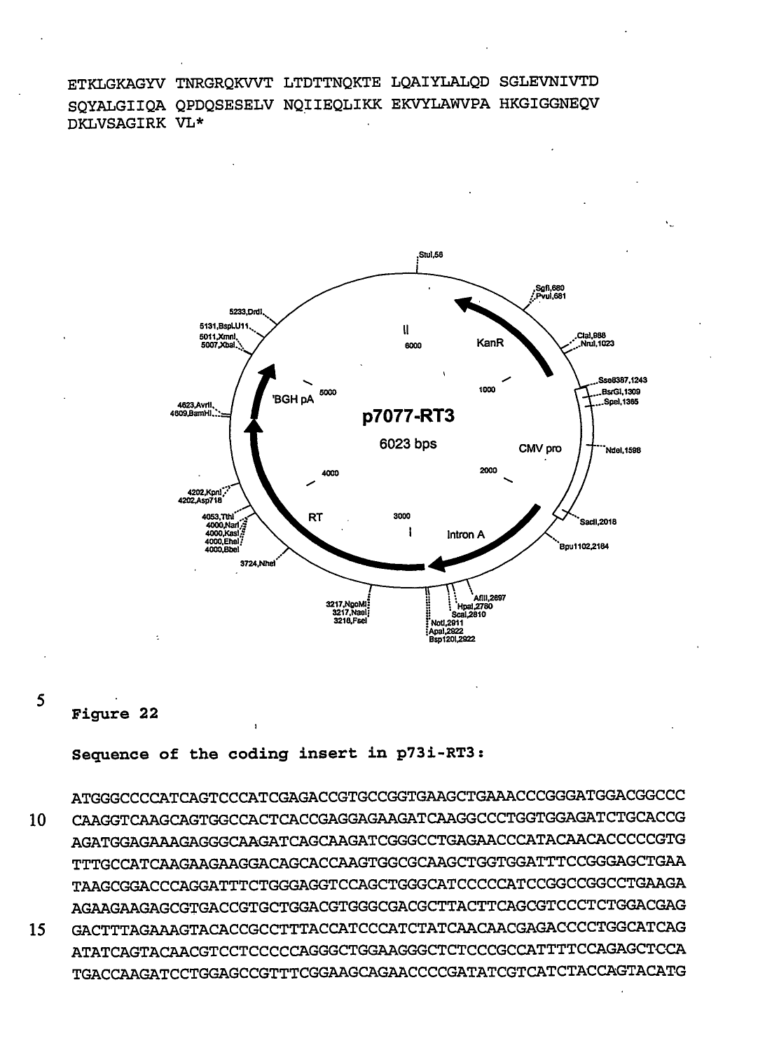

A standard plasmid backbone, pWRG7074 was developed. This backbone was used as the precursor plasmid from which to engineer pWRG7128, the HBsAg expression vector used in several clinical trials. In this section, the derivation of this backbone is described in narrative and shown in FIGS. 15 AND 16 , “Flowchart Derivitization of Plasmids PJV7074 and PJV7128” and ′Key Plasmid Feature Maps”, respectively. Briefly, pWRG7074 was derived by insertion of a single fragment containing the human CMV immediate early promoter and bovine growth hormone polyadenylation sequence into the standard, well characterized pUC19 bacterial plasmid vector. Several subsequent manipulations were employed to replace the ampicillin with a Kanamycin (Kan^{R}) resistance marker and to alter some restriction sites. The DNA fragment that was the source of the CMV promoter and bGH poly A region was obtained from plasmid pJW4303, a gift from Jim Mullins who was then at Stanford University.

Detailed narratives concerning the construction of plasmids WRG7074 and WRG7128 are given below. Unless noted otherwise, all cloning was performed at PowderJect Vaccines, Inc., Madison, Wis. (and formerly known as Agracetus, Inc., Auragen, Inc., or Geneva, Inc. Middleton, Wis.). Construction steps are in italics. Bullet points supply factual information regarding specific sequences.
step 1: The small HindII-BamHI fragment of pJW4303 (map, FIG. 10.2) containing the T?A signal peptide coding sequence was deleted by HindIll-BamHI digestion. This small fragment was replaced with a HindIII-Not1-BamnH1 linker to generate JW4303-Not1.

The fragment containing the CMV promoter and bGH poly A region from pJW4303 is a Sal I-Xho I fragment that PJV has since determined to be 2131 bp in length. The nucleotide sequence for the Sal I-Xho I fragment derived from pJW4303 has been deduced. The following items are identified in FIG. 10.2:
Sal I site at nucleotide position 1 of the fragment
Start of CMVIE promoter fragment at nucleotide position 7. This corresponds to nucleotide position 451 from GenBank sequence accession #M60321 (human cytomegalovirus immediate early protein gene 5′end).
End of CMVIE promoter fragment at nucleotide position 1648. This corresponds to nucleotide position 2097 from GenBank sequence accession #M60321 (human cytomegalovirus immediate early protein gene 5′end). It should be noted that a few nucleotide differences were observed between the deduced CMVIE promoter region and the above mentioned GenBank sequence. This is likely due to natural polymorphisms between different CMV virus isolates.
ATG translation initiation codon at nucleotide position 1661 for the signal peptide coding sequence of human tissue plasminogen activator. The TPA signal peptide coding sequence was derived from synthetic DNA as described by Lu et al. (J.

Virol. 70:3978, 1996). The Lu et al. publication briefly describes the construction of pJW4303, but this description contains some errors that are not consistent with the deduced sequence.
Coding sequence insertion sites Hind III and Nhe I at nucleotide positions 1649 and 1724, respectively. Note that the SWV nef homology region is shown as part of the bGHpA region.
Bam HI restriction site at nucleotide position 1741 that begins the region of homology to SIV nef. This corresponds to nucleotide position 9444 of GenBank sequence accession #M33262 (simian immunodeficiency virus, isolate 239, complete proviral genome and flanking sequence).
Bgl II restriction site at nucleotide position 1849 that terminates the SIV nef homology region. This corresponds to nucleotide position 9552 of GenBank sequence accession #M33262 (simian immunodeficiency virus, isolate 239, complete proviral genome and flanking sequence).
In 1999 it was discovered that a sequence representing 109 base pairs homologous with a sequence of the Simian Immunodeficiency Virus nef gene sequence, was present in this vector. As shown in FIG. 10.1, this sequence had been removed at the time pWRG7128 was constructed. However, it remained in pWRG7074. This sequence was present in pWG4303 and derivatives through pWRG7077 and pWRG7074. The SIV nef homology is found between nucleotide positions 77 and 184. Thus, the insertion of the SIV nef fragment adjacent to the bHG poly A region was an apparent construction artifact that occurred prior to PJV's receipt of the source DNA.
Start of bovine growth hormone poly A region homology at nucleotide position 1873. This corresponds to nucleotide position 2326 of GenBank sequence accession #M57764 (bovine growth hormone gene, complete coding sequence).
End of bovine growth hormone poly A region homology at nucleotide position 2096 of Attachment 4. This corresponds to nucleotide position 2550 of GenBank sequence accession #M57764 (bovine growth hormone gene, complete coding sequence).
Xho I restriction site at the end of the fragment (nucleotide position 2131).
step 2: Insertion of the CMV promoter and bGH poly A fragment from pJW4303-NotI (SalI-XhoI fragment) into the Sal I site of pUC19 yielding plasmid pWRG7012 (figure below).

pWRG7012 contains two Bamill sites and two HindIf sites. One of each of these sites was removed in subsequent steps (see below).
step 3: Deleted the EcoR1-Xba1 region of pWRG7012 to remove a large section of pUCI9's multiple cloning site, to generate pWRG7013 (FIG. 10.2).

PWRG7013 retains two HindIII sites but has only one BamHI site.
step 4: Removed the HindIII site, located 5′ of the CMV promoter, from pWRG7012 to allow easy utilization of the HindIII site between the intron and downstream inserts. This generated pWRG7014 (FIG. 10.2).

pWRG7014 retains two BamHI sites but has only one HindIII site.
step 5: To yield a plasmid containing only 1 HindIII site and 1 BamHI site, the HindIII-EcoR1 fragment from pWRG7013 was placed into HindIII-EcoR1 deleted pWRG7014 to generate pWRG7020, the ampicillin resistant version of WRG7077 (FIG. 10.2).
step 6: Deleted the Eam1105 1-Pst1flanked ampicillin resistance gene in pUC19. Blunt-ended the fragment containing the origin of replication by treatment with polymerase. Isolated the Pst1flanked kanamycin resistance gene in PUC4K. This fragment was blunt-ended by treatment with polymerase and ligated to the origin of replication fragment. This generated pWRG7072, a KanR vector that could accept the CMV-HBsAg-bGH-pA cassette from pWRG7031 (Step 8).
step 7: Deleted the Hd3-BamHl sequences of the polylinker in pWRG7020 and blunt-ended the vector with polymerase. Isolated the BamH1 flanked 1.4KB HBsAg containing fragment in pAM6. This fragment was blunt-ended by treatment with polymerase and ligated into the vector. This yielded pWRG7031, an ampicillin resistant HBsAg expression plasmid.
step 8: Deleted the Pvu2- Sph1 sequences of pWRG7072. Cut pWRG7031 with EcoR1, blunt-ended the site with polymerase, and further cut the plasmid with Sph1 and isolated the fragment containing the CMV, HBV, and bovine sequences. This fragment was ligated into the prepared pWRG7072 to yield pWRG7074.
step 9: Cut pWRG7074 with Bgl2, blunt-ended with polymerase, and further cut with BstX1 to make a vector fragment. pWRG7074 was cut with Nco1, blunt-ended with mung bean nuclease, and further cut with BsLX1 to make an insert fragment containing the 3′-enhancer. The ligation of these two fragments resulted in pWRG7128, a HBsAg expression plasmid devoid of the 5′-coding region of the HbxAg and the SIV NEF sequence found in pWRG7074.
step10: Construction of pWRG7077: Cut pWRG7072 with Sap1, blunt-ended with polymerase, and further cut with Sph1 to generate a fragment containing the origin of replication and kanamycin resistance gene. Cut and blunt-ended the EcoR1site in WRG7020, then partially cut with Sph1 to generate a fragment containing the CMV promoter, intron A, and BGH polyadenylation region. These fragments were ligated together to generate pWRG7077. The final vector pWRO7077 contains the original CMV-intron A-bGH-pA region derived from the source plasmid pJW4303 except with the alteration described in step 1 in which the TPA signal peptide coding sequence was replaced with a linker containing a Not I restriction site.

### EXAMPLE 8

We prepared copies of E6 and E7 by PCR from an HPV 16 genomic clone obtained from ATCC. The full length plasmid is maintained under BSL-2 conditions since it contains a complete viral genome. We detoxified E6 and E7 by deleting the binding regions for p53 and Rb respectively (Slebos et al., Virol. 1995, 208, 111-120; and Smahel et al., Virol. 2001, 281 231-238). PCR fragments were cloned into PJV7563 placing the genes under control of the CMV promoter without intron A.

Plasmids were prepared by Qiagen endotoxin free Mega kits and coated onto gold particles at 2 ug DNA per mg gold using the standard spermidine CaCl₂ method. Cartridges were prepared using 0.05 mg/ml PVP. B6 mice were immunized with single deliveries on the shaved abdomen using an XR research device at 500 psi.

TC-1 cells were obtained from Johns Hopkins School of Medicine. Cells were expanded in culture for a minimum number of passages and vials were then frozen and stored in liquid N₂ until use. Cells were prepared for injection in PBS. Anesthetized mice were injected subcutaneously with between 2×10⁴ to 2×10⁵ cells in 50 to 100 ul on the shaved right flank. Tumors were measured Mon., Wed., and Fri. beginning at day 7. Two diameter measurements at right angles were taken and multiplied to produce a square area. The health of the animals was also monitored. Mice were euthanized if tumors grew to greater than 120 mm², if tumors appeared necrotic, or mice appeared moribund.

For ELISPOT assays, mice were sacrificed one week after last immunization. Spleens were removed aseptically and single cell suspensions were prepared. Cells were plated at 1×10⁶ or 5×10⁵ cells per well in BD (-IFN ELISPOT kits according to manufacturers instructions. Peptides specific for E7 CD4 and CD8 and E6 CD8 were added at a final concentration of 10 uM. Media wells contained equivalent amounts of DMSO as wells containing peptide. Specific spots were calculated by subtracting media spots from those induced in the presence of peptide.

Results

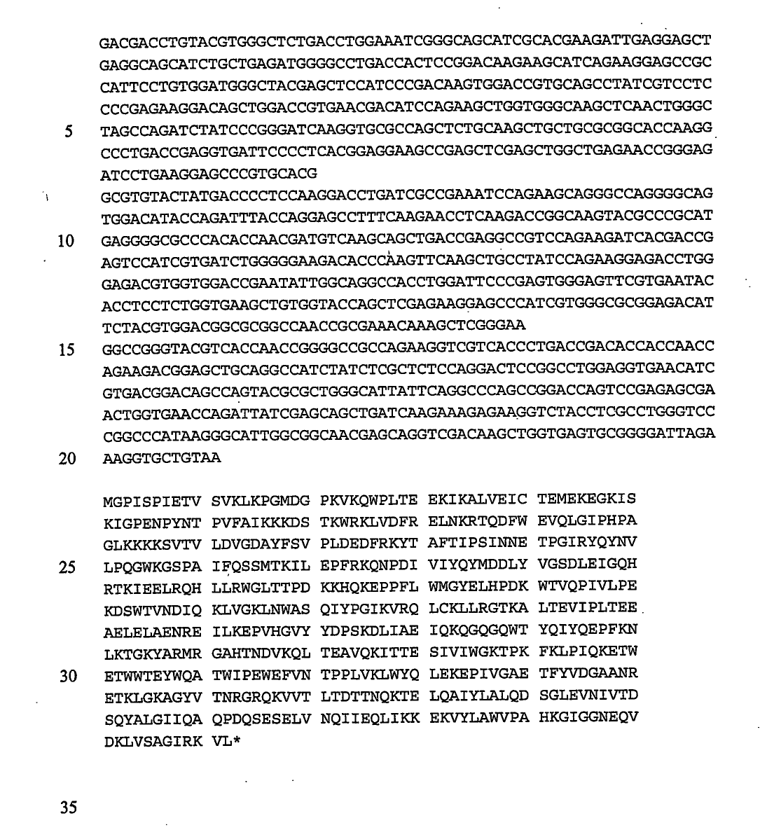

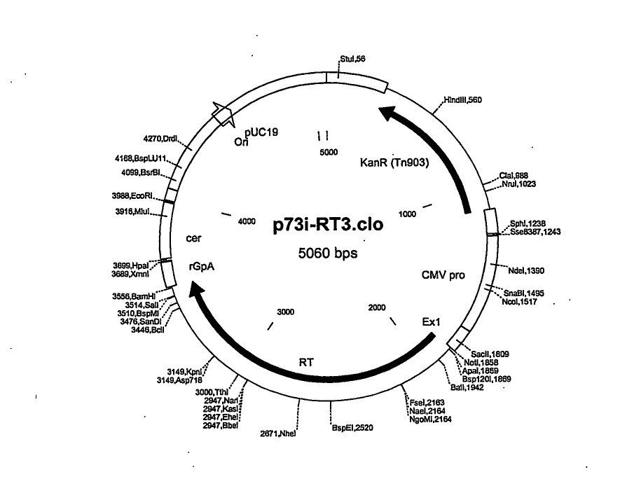

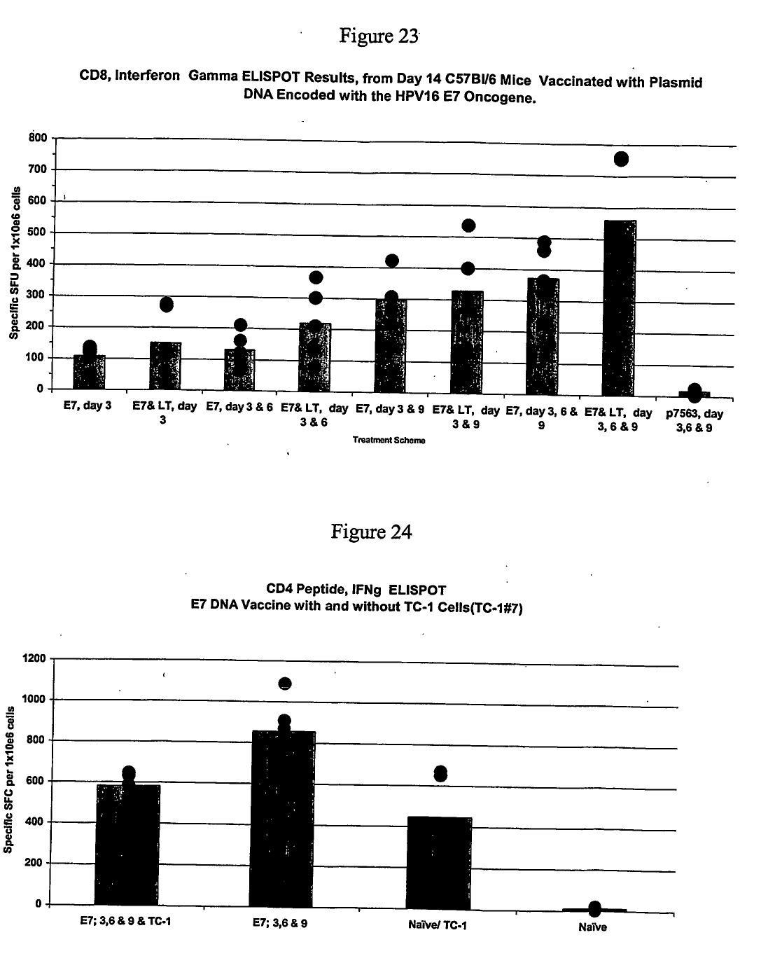

Immunization of B6 mice with either E6 or E7 DNA leads to the induction of significant numbers of (-IFN secreting cells (FIG. 23). In the case of E6, only a CD8-specific epitope is known. In the case of E7, both CD4- and CD8-specific epitopes have been identified and strong responses to both are seen after PMED immunization. Addition of either E. coli heat labile toxin (LT) or cholera toxin (CT) DNA to the E6 or E7 vaccines was tested. While LT increased (-IFN ELISPOT responses to E7 peptides approximately two-fold (FIG. 23), no increase in tumor protection was seen with either toxin.

Interestingly, injection of TC-1 cells alone, induces (-IFN responses to E6 and E7 (FIG. 24). These are lower than that observed with PMED immunization of three doses delivered in a cluster three days apart. Combining TC-1 injection with PMED immunization leads to an intermediate result.

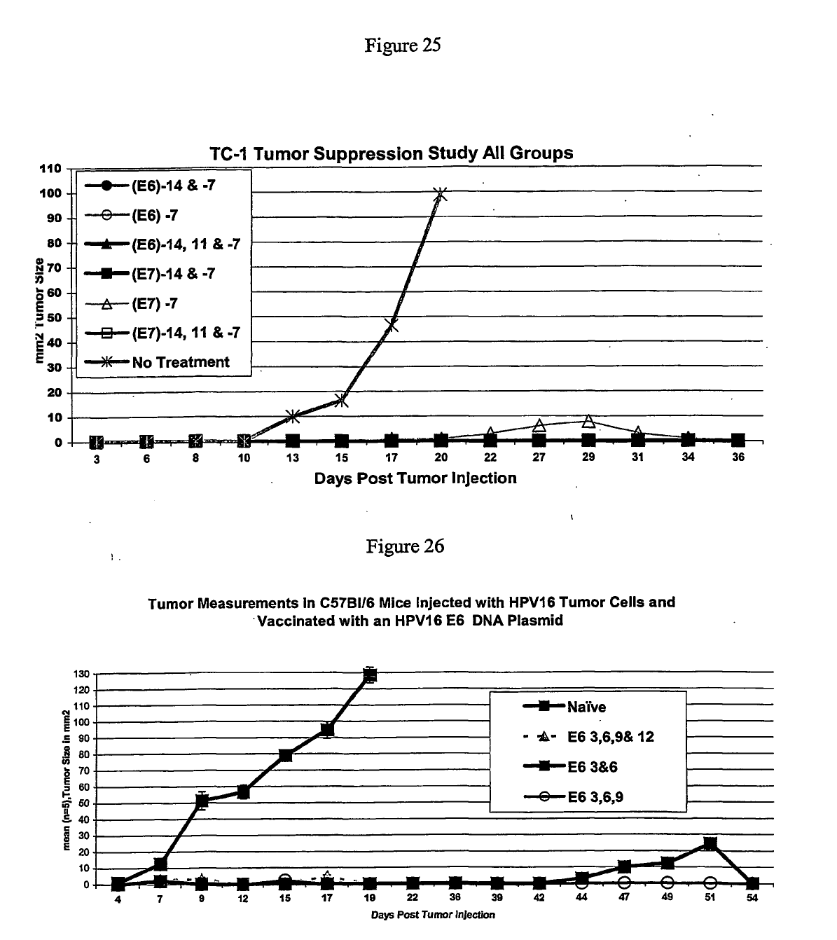

Injection of 5×10⁴ TC-1 cells leads to consistent and rapid development of tumors in untreated B6 mice. Prophylactic treatment with as little as one dose of either E6 or E7 DNA provides substantial protection against tumor development (FIG. 25). Therapeutic immunization with either E6 or E7 is also effective if delivered as a cluster of three doses beginning on day 3 after tumor injection (FIG. 26). Delivery of fewer doses is less effective (data not shown). Co-delivery of E6 and E7 plasmids was not more effective than either one alone, although this has only been tested once so far.

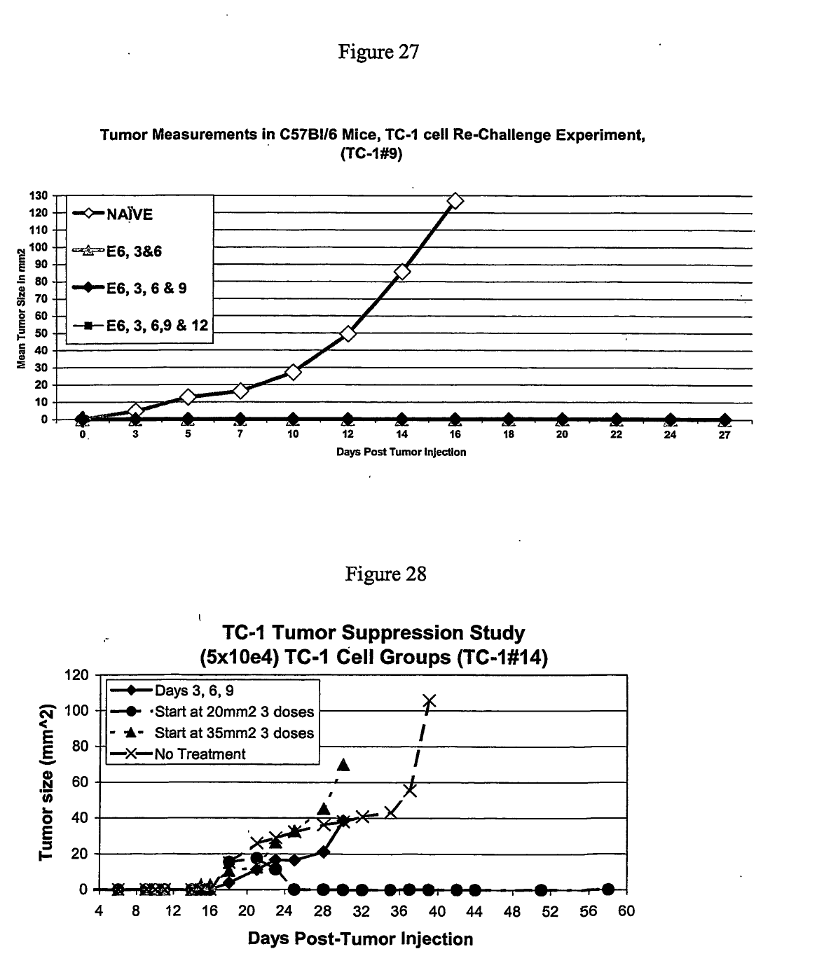

In one study, animals that were protected against an initial TC-1 challenge by E6 vaccination (FIG. 26) were rechallenged 50 days later without further treatment. As shown in FIG. 27, all animals were completely protected against this second challenge while all of the age-matched untreated controls developed tumors and were euthanized.

We have attempted treatment of larger tumors with varying degrees of success. For example, as shown in FIG. 28, treatment with a cluster of three doses of E6 DNA beginning at 20 mm² led to regression in 5 of 5 mice while treatment beginning at 35 mm² caused only a slight delay in tumor growth followed by rapid progression.

The data presented above indicate that detoxified E6 and E7 DNA elicit a strong T cell response against the respective peptide epitopes when delivered to B6 mice. Furthermore, these responses largely correlate with the ability of the vaccines to inhibit the growth of TC-1 tumor cells. Such treatment is effective as either prophylaxis or therapy.

### EXAMPLE 9

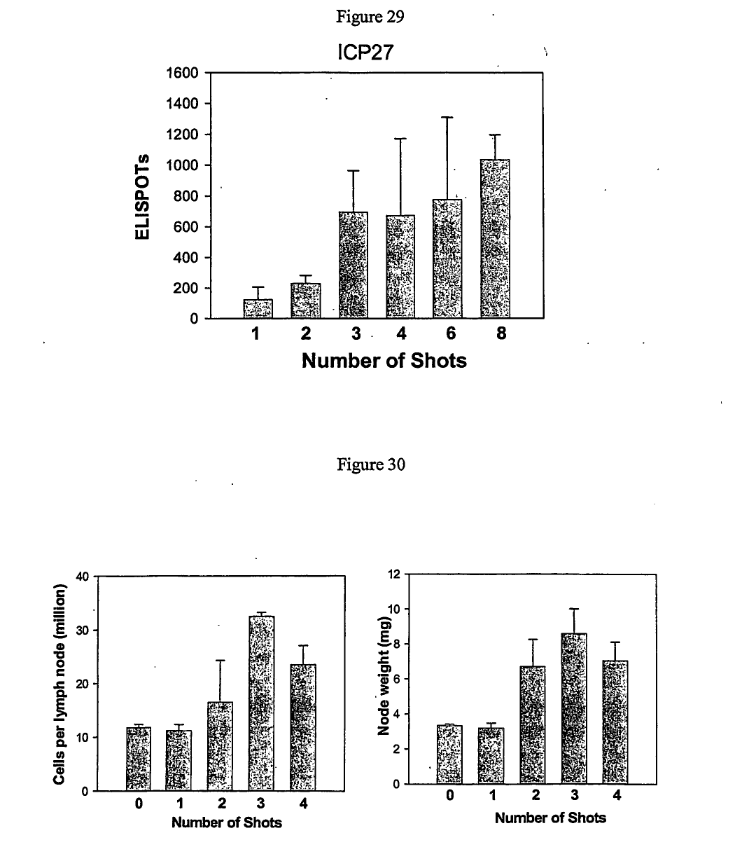

Mice were given from 1-8 shots using the ICP27 single gene plasmid and an interval of 2 days such that the final delivery for each group occurred on the same day. Two weeks following the final delivery CD8 ELISPOTs were measured. Results are shown in FIG. 29 and indicate that at least 3 deliveries are needed to gain near maximal effects with additional deliveries giving little or no improvement.

To examine cumulative effects of cluster dosing on lymph nodes mice were given 1,2,3 or 4shots of pPJV7630 (using 4 day intervals between shots) and then cells from the lymph nodes examined 8 days later. Results are shown in FIG. 30. With increasing number of shots, increases in the size of nodes was apparent. Weights and cell numbers in lymph nodes measured 8 days after completion of vaccine delivery were increased over naïve mice when more than I shot was given with 3 shots giving the highest values.

### EXAMPLE 10

Experiments were performed to investigate the effect of a boosting vaccination. Mice given a priming administration were compared to mice given both priming and boosting administrations. Both groups of mice were primed at the same time, and the same vaccines were used for vaccination. The second group was boosted 28 days after priming.

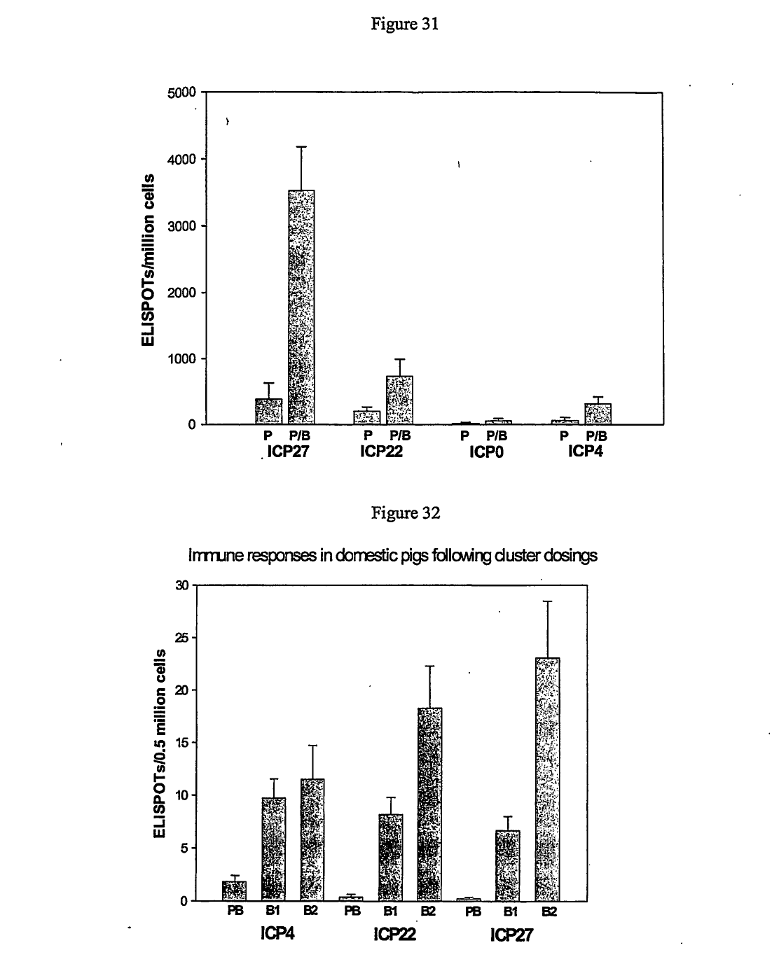

FIG. 31 shows the results from IFN-γ ELISPOT assay done on animals given pPJV7630 in a single cluster (P) or two clusters separated by 28 days (P/B). Splenocytes were tested using peptide libraries of each of the 4 immediate early antigens expressed from the pPJV7630 construct. Assays were done 2 weeks after final deliveries of vaccines. As can be seen performing a booster administration causes a substantial increase in the response which is stimulated.

### EXAMPLE 11

Domestic pigs were administered with PJV7630 by the XR1 device. Pigs were administered with two doses for each immunization and had a cluster schedule of 4 immunizations over a one-week period. Thus, each pig was given a total of 8 doses of vaccine in a cluster. Two cluster immunization boosts were initiated 21 days after the end of the previous cluster. Blood samples were taken prior to start of dosing (PB) and also 7-10 days after each boost (B1 and B2). IFN-γ ELISPOT assays were carried out as described and values are the average ± SEM for 10 animals. FIG. 32 shows the results that were obtained, and the effect of the boosting vaccination.

### EXAMPLE 12

Skin samples taken at 2, 3 and 4 days post PMED of a single shot of pPJV7630 with XR-1 were frozen, ground up and cytokine levels in the supernatant were tested. Using a CBA kit to evaluate inflammatory cytokines it found strong increases in IL-6, TNF-α and MCP-1 (monocyte chemotactic protein). These were highest on day 2 and decreased with time. No increases of IL-10, IL-12 or IFN-γ were found at any time point. The enhanced cytokines are commonly found in wound healing.

To examine cumulative effects of cluster dosing on the skin and lymph nodes mice were given 1, 2, 3 or 4 shots of pPJV7630 (using 4 day intervals between shots) and then cells from the lymph nodes examined 4 and 8 days later. With increasing number of shots, increases in the size of nodes was apparent. Weights and cell numbers in lymph nodes measured 8 days after completion of vaccine delivery were increased over naive mice when more than 1 shot was given (see FIG. 30).

Lymph node cells were also stained for MHC-il positive (antigen presenting cells), CD80 positive (activation marker) and double positive cells and analyzed by Flow cytometry (in Tables below). In general the number of single and double positive cells increased with shot number.

Day 4 - node populations
(% of total cells)

| | MHC-II | CD80 | MHC-II/CD80 |
|---|---|---|---|
| naive | 13 | 0.9 | 1.7 |
| 1 shot | 13.1 | 0.9 | 1.5 |
| 2 shots | 18.1 | 1.6 | 2.2 |
| 3 shots | 17.8 | 2.2 | 3.1 |
| 4 shots | 21.3 | 2.5 | 4.1 |

Day 8 - node populations
(% of total cells)

| | MHC-II | CD80 | MHC-II/CD80 |
|---|---|---|---|
| naive | 17 | 0.3 | 1.4 |
| 1 shot | 16.3 | 0.3 | 1 |
| 2 shots | 23 | 0.4 | 1.5 |
| 3 shots | 23 | 0.8 | 2.9 |
| 4 shots | 18 | 0.8 | 1.2 |

The analysis of lymph node populations indicates an approximate 5-10 fold increase in the number of antigen presenting cells in the lymph node as a result of clustering immunlzations.

In summary, radical physical changes are found in different regions of the shot site on the days following PMED. We have found inflammatory cytokines peak in the skin on day 2 following PMED. As well, during a cluster dosing an accumulation of cells, specifically activated antigen presenting cells, is found in the lymph nodes suggesting a heightened responsiveness of the skin.

### EXAMPLE 13

Balb/c mice were used for the experiments described. An area of abdomen was shaved and a primary immunisation of DNA was administered using particle mediated immunotherapeutic delivery (PMID). Each animal received a total of 3 μg DNA. This was administered either by conventional “pulse” immunisation (3×1 μg DNA) on either Day 0 or Day 4 or by a “cluster” immunisation with 1 μg DNA administered on each of alternate days (0, 2 and 4). Mice were culled 10 days after the first DNA administration and spleens were collected. The splenocytes were harvested by teasing out the spleen cells and erythrocytes were lysed. The splenocytes were washed and counted. Specialised ELIspot plates (coated with interferQn-gamma capture antibody and blocked) were used. Splenocytes were transferred to these plates and incubated overnight at 37° C./5% CO₂ in the presence of specific peptides. The splenocytes were lysed and the plate developed using standard procedures to demonstrate the number of interferon-gamma secreting cells present.

Results

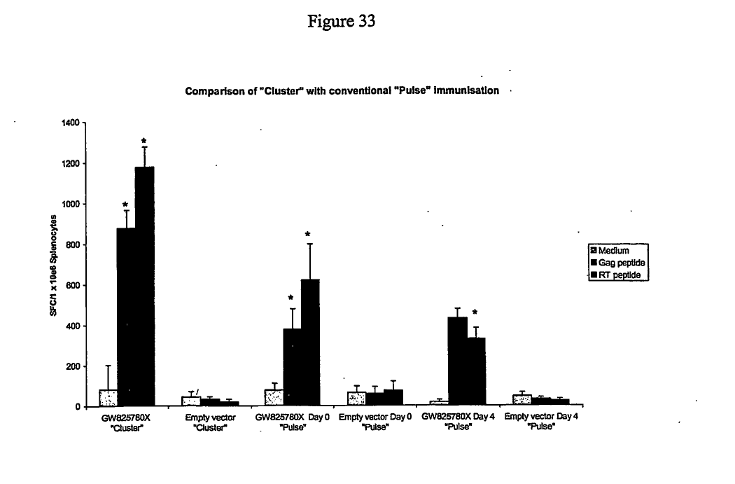

The results (shown in FIG. 33) indicated that there were significantly higher numbers of IFN-gamma spot forming cells isolated from the animals that had received the “cluster” immunisation compared with those that had received the same amount of DNA using the conventional “pulse” method. (* denotes significant differences)

The cellular immune response of mice immunised with a construct expressing Gag and RT antigens from HIV by the “cluster” method was significantly higher than immunising animals with the same amount of DNA using the conventional “pulse” method.

### EXAMPLE 14

Balb/c mice were used for the experiments described. An area of abdomen was shaved and a primary immunisation of DNA was administered using particle mediated immunotherapeutic delivery (PMID). Each animal received a total of 1 μg DNA. This was administered either by conventional “pulse” immunisation (2×0.5 μg DNA) on Day 0 or by a “modified cluster” immunisation with 0.5 μg DNA administered on each of day 0 and 7. All mice were boosted using a “pulse” of 1.0 μg DNA 83 days after the primary immunisaton. Mice were culled 7 days after the boost immunisation (Day 90) and spleens were collected. The splenocytes were harvested by teasing out the spleen cells and erythrocytes were lysed. The splenocytes were washed and counted. Specialised ELIspot plates (coated with interferon-gamma capture antibody and blocked) were used. Splenocytes were transferred to these plates and incubated overnight at 37° C./5% CO₂ in the presence of specific peptides. The splenocytes were lysed and the plate developed using standard procedures to demonstrate the number of interferon-gamma secreting cells present.

Results

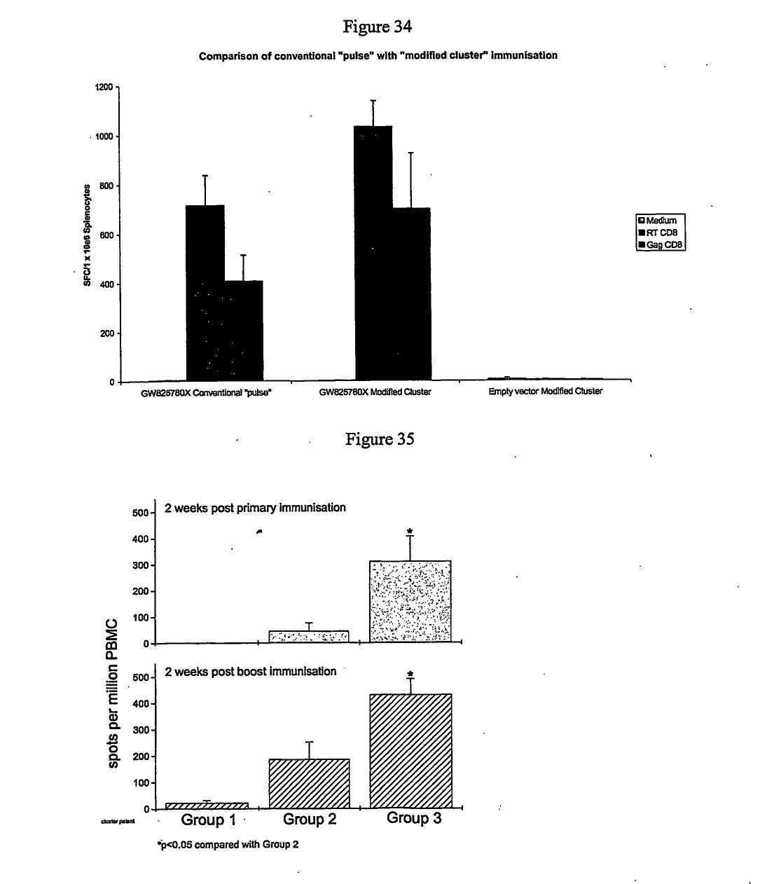

The results (shown in FIG. 34) indicated that there were higher numbers of IFN-gamma spot forming cells isolated from animals that had been immunised using a “modified cluster” compared with those that had received the same amount of DNA using the conventional “pulse” method. Thus the cellular immune response of mice immunised with a construct expressing Gag and RT antigens from HV by the “modified cluster” method was higher compared with that of animals immunised with the same amount of DNA using the conventional “pulse” method.

### EXAMPLE 15

The plasmid used expressed the HIV antigens RT, Nef and Gag. Preparation of cartridges for PMID was as previously described (Eisenbraun et al DNA and Cell Biology, 1993 Vol 12 No 9 pp 791-797; Pertner et al). Briefly, plasmid DNA was coated onto 2 μm gold particles (DeGussa Corp., South Plainfield, N.J., U.S.A.) and loaded into Tefzel tubing, which was subsequently cut into 1.27 cm lengths to serve as cartridges and stored desiccated at 4° C. until use. In a typical vaccination, each cartridge contained 0.5 mg gold coated with ˜1 μg DNA.

Groups of 4 minipigs received primary immunisation by PMID (initiated on day 1) followed by boost immunisation by PMID (initiated on day 57) into the ventral abdomen. Control animals were not immunised. Immunisation was either by pulse dosing (ie. 4 cartridges delivered on one occasion) or by cluster dosing (ie. 2 cartridges delivered on each of 3 occasions 48 hours apart). Fourteen days after initiation of primary or boost immunisation peripheral blood samples were collected for preparation of peripheral blood mononuclear cells (PBMC).

Porcine blood was collected into heparin, diluted 2:1 in PBS and layered over Histopaque (Sigma) in 50 ml Falcon tubes. The tubes were centrifuged at 1200 g for 30 minutes and the porcine lymphocytes harvested from the interface. Residual red blood cells were lysed using ammonium chloride lysis buffer. Cells were counted and resuspended in complete RPMI medium at 2×10⁶/ml.

In order to carry out the Elispot assay plates were coated with 8 μg/ml (in PBS) (purified mouse anti-swine IFN-γ, Biosource ASC4934). Plates were coated overnight at 4° C. Before use the plates were washed three times with PBS and blocked for 2 hours with complete RPMI medium. PBMC were added to the plates at 2×10⁵ cells/well. Total volume in each well was 200 μl. Recombinant Gag, Nef or RT protein (prepared in-house) was added at a final concentration of 5 μg/ml. Plates were incubated for 16 hours′in a humidified 37° C. incubator.

Cells were removed from the plates by washing once with water (with 1 minute soak to ensure lysis of cells) and three times with PBS. Biotin-conjugated anti-porcine IFN-γ was added at 0.5 μg/ml in PBS. Plates were incubated with shaking for 2 hours at room temperature. Plates were then washed three times with PBS before addition of Streptavidin alkaline phosphatase (Caltag) at 1/1000 dilution. Following three washes in PBS spots were revealed by incubation with BCICP substrate (Biorad) for 15-45 mins. Substrate was washed off using water and plates were allowed to dry. Spots were enumerated using the AID Elispot reader (Cadama Biomedical, UK).

Results

The results are shown in FIG. 35. After a primary immunisation the number of IFN-γ producing spots in PBMC from minipigs primed by cluster immunisation was significantly greater compared with those that received pulse immunisation (311±96 and 45±31 mean ±SEM, respectively; p<0.05 Student's t test). Furthermore, the number of IFN-γ producing spots following a pulse boost was significantly greater in animals that received a cluster prime compared with a pulse prime (431±60 and 186±66 mean±SEM, respectively; p<0.05 Student's t test). In summary, these results show that cluster priming provides an advantage over the conventional pulse priming and that this advantage is sustained into the subsequent boosting phase of the immune response.

In the Figure Group 1 is the control non-immunised; Group 2 is the pulse prime pulse boost group;Group 3 is the pulse prime, cluster boost group.

### EXAMPLE 16

C57BL/6 mice were used for the experiments described. An area of abdomen was shaved and a primary immunisation of DNA was administered using particle mediated immunotherapeutic delivery (PMID). Each animal received a conventional “pulse” immunisation (1 μg DNA ovalbumin) on either Day 0 or by a “cluster” immunisation with 1 μg DNA administered on each of alternate days (0, 2)-cluster 2X or days 0,2 and 4-cluster3X. Mice were culled 10 days after the first DNA administration and spleens were collected. The splenocytes were harvested by teasing out the spleen cells and erythrocytes were lysed. The splenocytes were washed and counted. Specialised ELIspot plates (coated with interferon-gamma or IL2 capture antibody and blocked) were used. Splenocytes were transferred to these plates and incubated overnight at 37° C./5% CO₂ in the presence of specific peptides. Previously defined ovalbumin specific CD4 and CD8 peptides were used. splenocytes were lysed and the plate developed using standard procedures to demonstrate the number of interferon-gamma or IL2 secreting cells present.

Results

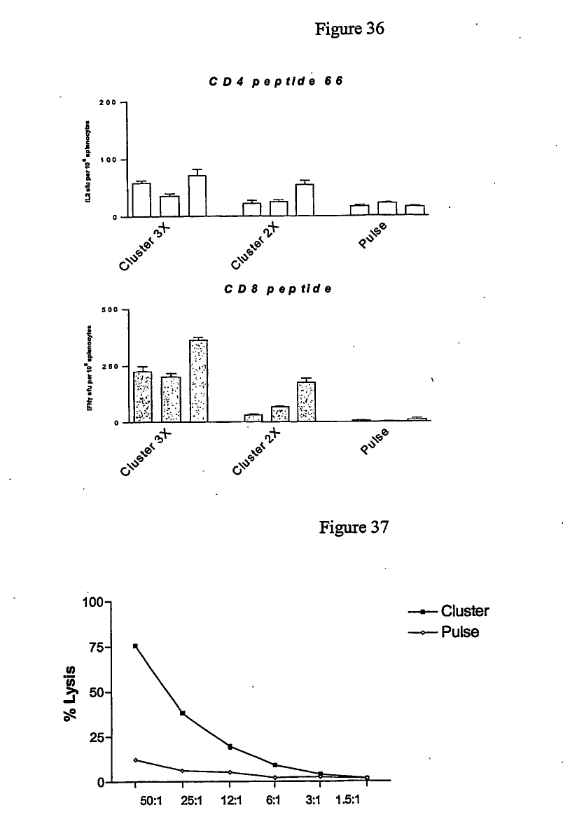

The results (shown in FIGS. 36 and 37) indicated that there were significantly higher numbers of IFN-gamma and IL2 spot forming cells isolated from the animals that had received the “cluster” immunisation compared with those that had received the same amount of DNA using the conventional “pulse” method. In FIG. 36 each bar represents the response from an individual mouse.

The cellular immune response of mice immunised with a construct expressing ovalbumin by the “cluster” method was significantly higher than immunising animals with the same amount of DNA using the conventional “pulse” method.

### EXAMPLE 17

C57BL/6 mice were used for the experiments described. An area of abdomen was shaved and a primary immunisation of DNA was administered using particle mediated immunotherapeutic delivery (PMID). Each animal received a total of 3 μg DNA. This was administered either by conventional “pulse” immunisation (3×μg DNA) on either Day 0 or by a “cluster” immunisation with 1 μg DNA administered on each of alternate days (0, 2 and 4).

Animals which has been primed with either a pulse or cluster immunisation were then boosted 29 days later with a single pulse immunisation of 1 ug DNA. Spleens were removed 9 days after the boost (day 38) as described above and the frequency of antigen specific cells determined by ELISPOT. In addition the ability of CD8 T cells to kill antigen specific targets was determined by a Europium based CTL assay, following 5 days in vitro expansion with peptide or IL2.

Results

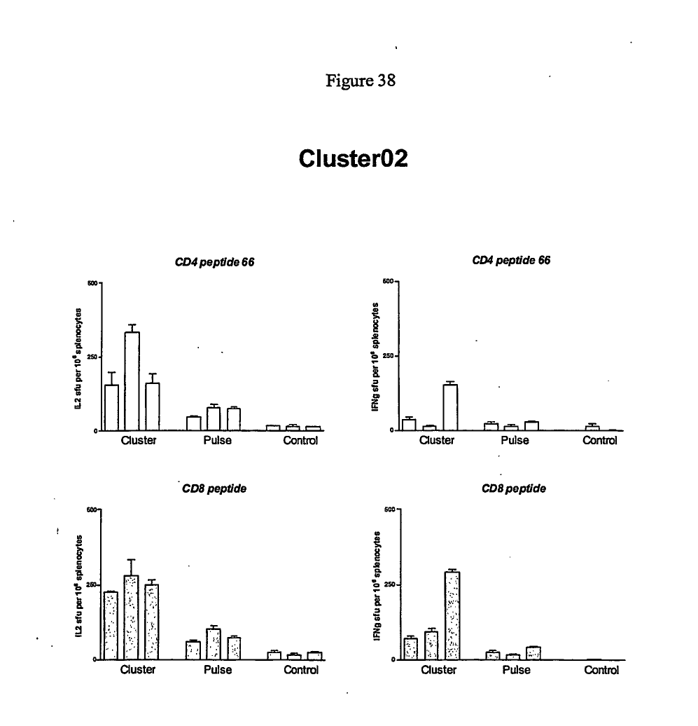

The results shown in FIG. 38 indicate that animals primed with a cluster immunisation showed a stronger recall response than mice immunised with the same dose of DNA but as pulse immunsation. This was shown by the increase in the frequency of IFNg and IL2 producing cells by ELISPOT. Animals primed by cluster immunisation also showed a stronger CTL response compared with animals immunised with pulse immunisation following a pulse DNA boost.

The memory immune response of mice immunised with a construct expressing ovalbumin by the “cluster” method was significantly higher than immunising animals with the same amount of DNA using the conventional “pulse” method.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be covered by the present invention.

1. A method of eliciting a T cell response against a T cell epitope in a host mammalian subject, which method comprises (i) a first immunisation that comprises at least two administrations which are from 1 to 14 days apart to the subject, wherein each administration comprises administering a nucleotide sequence of interest (NOI) encoding the T cell epitope, and optionally (ii) a second immunisation that comprises at least one administration to the subject of (a) a NOI encoding the T cell epitope, or (b) a protein comprising the T cell epitope, wherein the time between the first administration of the first immunisation, and the first administration of the second immunisation, is from 21 to 365 days.

2. A method according to claim 1 wherein the administrations of the first and/or second immunisation occur over from 2 to 12 days.

3. A method according to claim 1 or wherein in the first and/or second immunisation the NOI or protein is administered from 2 to 10 times.

4. A method according to claim 1, wherein 2, 3, 4 or more of the administrations of the first and/or second immunisation are from 2 to 6 days apart.

5. A method according to claim 1, wherein the time between the first administration of the first immunisation and the first administration of the second immunisation is from 50 to 250 days.

6. A method according to claim 1, which further comprises a third immunisation that comprises at least one administration to the subject of (a) a NOI encoding the T cell epitope, or (b) a protein comprising the T cell epitope, wherein the time between the first administration of the second immunisation, and the first administration of the third immunisation, is from 10 to 365 days.

7. A method according to claim 6 wherein in the third immunisation: (i) the NOI or protein is administered 2 to 5 times, and/or (ii) the administrations are from 2 to 6 days apart, and/or (iii) the time between the first administration of the second immunisation and the first administration of the third immunisation is from 50 to 250 days.

8. The method according to claim 1, wherein the NOI comprises a DNA sequence under the control of a regulatory sequence capable of directing expression of the DNA sequence in a cell of the subject.

9. The method according to claim 1, wherein the T cell epitope is a CD4+helper T lymphocyte cell epitope and/or CD8+T lymphocyte (CTL) epitope.

10. The method according to claim 1, wherein one or more of the administrations of NOI comprise administration of from 0.1 to 2 ug of NOI.

11. The method according to claim 1, wherein one or more of the administrations comprises administration to the skin.

12. The method according to claim 1, wherein for at least one of the administrations of NOI or protein, the NOI or protein is coated on, or incorporated in, a particle.

13. The method according to claim 12 wherein the particle is administered to the subject by a particle acceleration device.

14. The method according to claim 1, wherein for at least one of the administrations of NOI or protein, the NOI or protein is administered as (i) a pharmaceutical composition comprising a pharmaceutical acceptable carrier, excipient or diluent; or (ii) a vaccine composition comprising an immunologically acceptable carrier, excipient or diluent; and (iii) an immunotherapeutic composition comprising an immunologically acceptable carrier, excipient or diluent.

15. The method according to claim 1, wherein the NOI or protein is co-administered with an adjuvant or a polynucleotide that is capable of expressing an adjuvant in a cell of the subject; or a method in which the NOI or protein is administered as (i) a pharmaceutical composition comprising a pharmaceutical acceptable carrier, excipient or diluent; or (ii) a vaccine composition comprising an immunologically acceptable carrier, excipient or diluent; or (iii) an immunotherapeutic composition comprising an immunologically acceptable carrier, excipient or diluent, and wherein the composition further comprises an adjuvant or a polynucleotide that is capable of expressing an adjuvant in a cell of the subject.

16. The method according to claim 15 wherein the adjuvant is a non-toxic form of the E. coli heat- labile enterotoxin (LT) or the Vibrio Cholerae cholera toxin (CT).

17. The method according to claim 15 wherein the adjuvant is the B subunit (LTB) of the LT enterotoxin or B subunit (CTB) of CT cholera toxin.

18. The method according to claim 1, wherein the T cell epitope is from a pathogen or from a cancer cell.

19. The method according to claim 1, wherein the T cell epitope is from HSV, HIV, HPV, hepatitis virus or influenza virus antigens.

20. The method according to claim 1, which is carried out to prevent or treat a disease in the subject.

21. The method according to claim 1, wherein the NOI encodes at least two HSV, HIV, HPV, hepatitis virus or influenza virus antigens.

22. The method according to claim 21 wherein the NOI encodes an HIV-1 gag protein, or fragment containing a gag epitope thereof, and a second HIV antigen or a fragment encoding an epitope of said second HIV antigen.

23. The method according to claim 22 wherein the second antigen is selected from the group consisting of: Nef, RT or a fragment containing an epitope of Nef or RT.

24. The method according to claim 22 wherein the NOI encodes a combination of antigens selected from the group consisting of:
Gag (p17,p24), Nef truncate
Gag (p17,p24) (codon optimised), Nef (truncate)
Gag (p17,p24), RT, Nef (truncate)
Gag (p17,p24), codon optimised RT, Nef (truncate)
Gag (p17,p24), codon optimised RT, codon optimised Nef truncate;
and/or inactivated codon optimised RT, truncated Nef and p17/p24 portion of the codon optimised gag gene, optionally operatively linked downstream of an Iowa length HCMV promoter+exon 1, and upstream of a rabbit globin poly-adenylation signal.

25. The method according to claim 1, wherein at least two different NOI's are administered which each encode the same epitope and/or at least two different proteins are administered which comprise the same epitope.

26. An assay for testing the effectiveness of a method of eliciting a T cell response, wherein the method comprises (i) a first immunisation that comprises at least two administrations which are from 1 to 14 days apart to the subject, wherein each administration comprises administering a nucleotide of interest (NOI) encoding a T cell epitope, and optionally (ii) a second immunisation that comprises at least one administration to the subject of (a) a NOI encoding the T cell epitope, or (b) a protein comprising the T cell epitope, wherein the time between the first administration of the first immunisation, and the first administration of the second immunisation, is from 21 to 365 days, wherein the assay comprises carrying out the method on a mammalian subject and then determining the level of activated or memory T cells specific to the epitope in the subject.

27. An assay according to claim 26 which comprises determining whether (i) the administrations of the first immunisation all fall within the time period between the first administration of the first immunisation and the decline in the level of activated T cells to basal level, and/or (ii) the first administration of the second immunisation occurs after the decline in the level of activated T cells to basal level.

28. A kit for carrying out the method of claim 1 wherein the kit comprises: (a) a NOI encoding a T cell epitope or, a composition comprising the NOI or a protein, wherein the composition is (i) a pharmaceutical composition comprising a pharmaceutical acceptable carrier, excipient or diluent: or (ii) a vaccine composition comprising an immunologically acceptable carrier, excipient or diluent: or (iii) an immunotherapeutic composition comprising an immunologically acceptable carrier, excipient or diluent; and (b) instructions for administration of the NOI or composition in accordance with the methed.
